(19) United States
(12) Patent Application Publication
(10) Pub. No.: US 2025/0372196 A1
(43) Pub. Date: Dec. 4, 2025

(54) CHARACTERIZATION OF INTERACTIONS BETWEEN COMPOUNDS AND POLYMERS USING POSE ENSEMBLES

(71) Applicant: Atomwise Inc., San Francisco, CA (US)

(72) Inventors: Pawel Gniewek, San Francisco, CA (US); Bradley Worley, San Francisco, CA (US); Brandon Anderson, San Francisco, CA (US); Kate Stafford, San Francisco, CA (US); Henry van den Bedem, San Francisco, CA (US)

(21) Appl. No.: 18/861,122

(22) PCT Filed: Mar. 17, 2023
(86) PCT No.: PCT/US23/64667

§ 371 Date: Oct. 28, 2024

Related U.S. Application Data
(60) Provisional application No. 63/336,841, filed on Apr. 29, 2022.

(51) Int. Cl. G16B 15/30; G06N 3/045; G06N 3/0464; G06N 3/09; G16B 40/20

(52) U.S. Cl. CPC G16B 15/30; G06N 3/045; G06N 3/0464; G06N 3/09; G16B 40/20

(57) ABSTRACT

Systems and methods for characterizing an interaction between a compound and a polymer include obtaining a plurality of sets of atomic coordinates. Each set of atomic coordinates comprises the compound bound to the polymer in a corresponding pose in a plurality of poses. Each respective set of atomic coordinates, or an encoding thereof, is sequentially inputted into a neural network, to obtain a corresponding initial embedding as output, thereby obtaining a plurality of initial embeddings. Each initial embedding corresponds to a set of atomic coordinates in the plurality of sets of atomic coordinates. An attention mechanism is applied to the plurality of initial embeddings, in concatenated form, to obtain an attention embedding. A pooling function is applied to the attention embedding to derive a pooled embedding. The pooled embedding is inputted into a model to obtain an interaction score of the interaction between the compound and the polymer.

### CROSS REFERENCE TO RELATED APPLICATIONS

This application claims priority to U.S. Provisional patent application No. 63/336,841, entitled “CHARACTERIZATION OF INTERACTIONS BETWEEN COMPOUNDS AND POLYMERS USING POSE ENSEMBLES,” filed Apr. 29, 2022, which is hereby incorporated by reference.

### TECHNICAL FIELD

This application is directed to using models to characterize interactions between test compounds and target polymers.

### BACKGROUND

Fundamentally, biological systems operate through the physical interaction of molecules, such as a compound with a target polymer. Structure-based, virtual high throughput screening (vHTS) methods have been used to characterize interactions between candidate (test) compounds and a target polymer through machine learning approaches. Such characterization, for instance, can report a continuous or categorical activity label, a PKa, or any other suitable metric to characterize the interaction between a candidate compound and a target polymer.

One drawback with vHTS machine learning methods is that they do not adequately take into account enthalpic and entropic components of receptor-ligand complex formation. Structure-based deep learning methods typically predict bioactivity from docked, static ligand poses. However, these approaches ignore the entropic contribution to the change in free energy. Predicting bioactivity from an ensemble of docked poses can overcome this limitation, but it requires that the model recognizes and is sensitive to different poses. However, conventional machine learning methods tend not to be sensitive to different poses. FIG. 19 illustrates this insensitivity, where machine learning models such as convolutional neural networks incorrectly favor poses that have all the right components but are fundamentally incorrect overall. FIG. 18 illustrates a situation in which the pose on the left and the pose on right have the same parts, two eyes, two eyebrows, a nose, lips, and the overall shape of a head. Teaching the machine learning model that the pose on the left, therefore, is the correct one can prove to be difficult. Because of this, there is an inherent pose insensitivity in conventional vHTS machine learning methods. This pose insensitivity can lead to the incorrect or inaccurate characterization of the interaction between a test compound and a target polymer. For instance, this pose insensitivity can lead a vHTS machine learning approach, which provides a categorical activity label for each compound in a screening library, to incorrectly label a certain percentage of the compounds in the screening library.

Given the above background, what is needed in the art are methods for imposing pose sensitivity on vHTS machine learning methods so that such vHTS machine learning methods are sensitive to bad poses.

### SUMMARY

The present disclosure addresses the problems identified in the background by making use of vHTS machine learning models that predict bioactivity from multiple poses concurrently. The disclosed vHTS machine learning models conditional multi-task architecture enforces sensitivity to distinct ligand poses, and its architecture includes an attention mechanism to exploit hidden correlations in pose distributions. The disclosed vHTS machine learning models improve bioactivity prediction compared to baseline models that predict from static ligand poses alone.

Accordingly, one aspect of the present disclosure provides a computer system for characterizing an interaction between a test compound and a target polymer. The computer system comprises one or more processors and memory addressable by the one or more processors. The memory stores at least one program for execution by the one or more processors. The at least one program comprises instructions for obtaining a plurality of sets of atomic coordinates. Each respective set of atomic coordinates in the plurality of sets of atomic coordinates comprises the test compound bound to the target polymer in a corresponding pose in a plurality of poses. In some embodiments, each respective set of atomic coordinates in the plurality of sets of atomic coordinates comprises atomic coordinates for at least 30 atoms.

For each respective set of atomic coordinates in the plurality of sets of atomic coordinates, the at least one program comprises instructions for inputting the respective set of atomic coordinates or an encoding of the respective set of atomic coordinates into a first neural network, to obtain a corresponding initial embedding as output of the first neural network, thereby obtaining a plurality of initial embeddings. Each initial embedding in the plurality of initial embeddings corresponds to a set of atomic coordinates in the plurality of sets of atomic coordinates. The first neural network comprises more than 400 parameters.

The at least one program further comprises instructions for applying an attention mechanism to the plurality of initial embeddings, in concatenated form, thereby obtaining an attention embedding.

The at least one program further comprises instructions for applying a pooling function to the attention embedding to derive a pooled embedding.

The at least one program further comprises instructions for inputting the pooled embedding into a first model thereby obtaining a first interaction score of an interaction between the test compound and the target polymer. The first model comprises more than 400 parameters.

In some embodiments, the first interaction score represents a binding coefficient of the test compound to the target polymer. In some such embodiments the binding coefficient is an IC₅₀, EC₅₀, Kd, KI, or pKI for the test compound with respect to the target polymer.

In some embodiments, the first interaction score represents an in silico pose quality score of the test compound to the target polymer.

In some embodiments, the first model is a fully connected second neural network.

In some embodiments, the at least one program further comprises instructions for inputting the pooled embedding into a second model thereby obtaining a second interaction score of an interaction between the test compound and the target polymer. In such embodiments, the first model is a first fully connected neural network, the second model is a second fully connected neural network, the first interaction score represents an in silico pose quality score of the test compound to the target polymer, and the second interaction score represents an in silico pose quality score of the test compound to the target polymer. In some such embodiments, the at least one program further comprises instructions for inputting the first interaction score and the second interaction score into a third model to obtain a third interaction score, where the third model is a third fully connected neural network. In some such embodiments, the third interaction score is discrete-binary activity score with a first value when the test compound is determined by third model to be inactive and a second value when the test compound is determined by the third model to be active.

In some embodiments, the target polymer is a protein, a polypeptide, a polynucleic acid, a polyribonucleic acid, a polysaccharide, or an assembly of any combination thereof.

In some embodiments, each set of atomic coordinates in the plurality of sets of atomic coordinates comprises three-dimensional coordinates {x₁, . . . , x_{N}} for at least a portion of the polymer from a crystal structure of the target polymer resolved at a resolution of 2.5 Å or better or a resolution of 3.3 Å or better.

In some embodiments, each set of atomic coordinates in the plurality of sets of atomic coordinates comprises an ensemble of three-dimensional coordinates for at least a portion of the target polymer determined by nuclear magnetic resonance, neutron diffraction, or cryo-electron microscopy.

In some embodiments, the first interaction score is a binary score, where a first value for the binary score represents an IC₅₀, EC₅₀, Kd, KI, or pKI for the test compound with respect to the target polymer that is above a first threshold, and a second value for the binary score represents an IC₅₀, EC₅₀, Kd, KI, or pKI for the test compound with respect to the target polymer that is below the first threshold.

In some embodiments, the test compound satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

In some embodiments, the test compound is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons.

In some embodiments, the test compound is an organic compound having a molecular weight of between 400 Daltons and 10000 Daltons.

In some embodiments, the plurality of sets of atomic coordinates consists of between 3 and 64 poses. In some embodiments, the plurality of sets of atomic coordinates consists of between 2 and 64 poses.

In some embodiments, the first neural network is a convolutional neural network. In some such embodiments, each respective set of atomic coordinates in the plurality of sets of atomic coordinates comprises atomic coordinates for at least 80 atoms.

In some embodiments, the first neural network is a graph neural network. In some such embodiments, the graph neural network is characterized by an initial embedding layer and a plurality of interaction layers that each contribute an interaction data structure, in a plurality of interaction data structures, for each atom in the respective set of atomic set of atomic coordinates for the corresponding pose in the plurality of poses that are pooled to form the corresponding initial embedding for the corresponding pose.

In some embodiments, the first neural network is a equivariant neural network or a message passing neural network.

In some embodiments, the first neural network comprises a plurality of graph convolutional blocks and each block considers connectivity within the respective set of atomic coordinates using a plurality of radial graphs.

In some embodiments, the first neural network comprises 1×10⁶ parameters.

In some embodiments, the corresponding initial embedding comprises a data structure having between 128 and 768 values. In some embodiments, the corresponding initial embedding comprises a data structure having more than 100 values. In some embodiments, the corresponding initial embedding comprises a data structure having more than 80 values, more than 100 values, more than 120 values, more than 140 values, or more than 160 values. In some embodiments, the corresponding initial embedding comprises a data structure consisting of between 100 values and 2000 values.

In some embodiments, the plurality of initial embeddings comprises a first plurality of values, and the applying the attention mechanism comprises: (i) inputting the first plurality of values into an attention neural network thereby obtaining a first plurality of weights, where each weight in the first plurality of weights corresponds to a respective value in the first plurality of values, and (ii) weighting each respective value in the first plurality of values by the corresponding weight in the plurality of weights thereby obtaining the attention embedding. In some such embodiments, the first plurality of weights sum to one and each weight in the first plurality of weights is a scalar value between zero and one.

In some embodiments, the pooling function collapses the attention embedding into the pooled embedding by applying a statistical function to combine each portion of the attention embedding representing a different pose in the plurality of poses to form the pooled embedding. In some such embodiments, the attention embedding includes a corresponding plurality of values for a corresponding plurality of elements for each respective pose in the plurality of poses and the statistical function is a maximum function that takes a maximum value across corresponding elements of each respective pose represented in the attention embedding to form the pooled embedding. In some embodiments, the attention embedding includes a corresponding plurality of values for a corresponding plurality of elements for each respective pose in the plurality of poses and the statistical function is an average function that averages the corresponding elements of each respective pose represented in the attention embedding to form the pooled embedding.

In some embodiments, the first model is a regression task and the first interaction score quantifies the interaction between the test compound and the target polymer.

In some embodiments, the first model is a classification task and the first interaction score classifies the interaction between the test compound and the target polymer.

Another aspect of the present disclosure provides a method for characterizing an interaction between a test compound and a target polymer. The method comprises obtaining a plurality of sets of atomic coordinates. Each respective set of atomic coordinates in the plurality of sets of atomic coordinates comprises the test compound bound to the target polymer in a corresponding pose in a plurality of poses. In some embodiments, each respective set of atomic coordinates in the plurality of sets of atomic coordinates comprises atomic coordinates for at least 30 atoms. Further in the method, for each respective set of atomic coordinates in the plurality of sets of atomic coordinates, the respective set of atomic coordinates, or an encoding of the respective set of atomic coordinates, is inputted into a first neural network to obtain a corresponding initial embedding as output of the first neural network. In this way a plurality of initial embeddings is obtained, where each initial embedding in the plurality of initial embeddings corresponds to a set of atomic coordinates in the plurality of sets of atomic coordinates. In some such embodiments the first neural network comprises more than 400 parameters. The method further comprises applying an attention mechanism to the plurality of initial embeddings, in concatenated form, thereby obtaining an attention embedding. The method further comprises applying a pooling function to the attention embedding to derive a pooled embedding. The method further comprises inputting the pooled embedding into a first model thereby obtaining a first interaction score of an interaction between the test compound and the target polymer. In some embodiments the first model comprises more than 400 parameters.

Another aspect of the present disclosure provides a non-transitory computer readable storage medium. The non-transitory computer readable storage medium stores instructions, which when executed by a computer system, cause the computer system to perform a method for characterizing an interaction between a test compound and a target polymer. The method comprises obtaining a plurality of sets of atomic coordinates. each respective set of atomic coordinates in the plurality of sets of atomic coordinates comprises the test compound bound to the target polymer in a corresponding pose in a plurality of poses. In some embodiments, each respective set of atomic coordinates in the plurality of sets of atomic coordinates comprises atomic coordinates for at least 30 atoms. The method further comprises, for each respective set of atomic coordinates in the plurality of sets of atomic coordinates, inputting the respective set of atomic coordinates or an encoding of the respective set of atomic coordinates into a first neural network to obtain a corresponding initial embedding as output of the first neural network. In this way a plurality of initial embeddings is obtained where each initial embedding in the plurality of initial embeddings corresponds to a set of atomic coordinates in the plurality of sets of atomic coordinates. In some embodiments the first neural network comprises more than 400 parameters. The method further comprises applying an attention mechanism to the plurality of initial embeddings, in concatenated form, thereby obtaining an attention embedding. The method further comprises applying a pooling function to the attention embedding to derive a pooled embedding. The method further comprises inputting the pooled embedding into a first model thereby obtaining a first interaction score of an interaction between the test compound and the target polymer. In some embodiments, the first model comprises more than 400 parameters.

### BRIEF DESCRIPTION OF THE DRAWINGS

In the drawings, embodiments of the systems and methods of the present disclosure are illustrated by way of example. It is to be expressly understood that the description and drawings are only for the purpose of illustration and as an aid to understanding, and are not intended as a definition of the limits of the systems and methods of the present disclosure.

FIGS. 1A and 1B illustrate a computer system in accordance with some embodiments of the present disclosure.

FIGS. 2A, 2B, 2C, 2D, 2E, and 2F illustrate methods for characterizing an interaction between a test compound and a target polymer in accordance with some embodiments of the present disclosure.

FIG. 3 is a schematic view of an example training compound in a pose relative to a target polymer in accordance with some embodiments of the present disclosure.

FIG. 4 is a schematic view of a geometric representation of input features in the form of a three-dimensional grid of voxels, in accordance with some embodiments of the present disclosure.

FIG. 5 and FIG. 6 are views of a compound encoded onto a two dimensional grid of voxels, in accordance with some embodiments of the present disclosure.

FIG. 7 is the view of the visualization of FIG. 6, in which the voxels have been numbered, in accordance with some embodiments of the present disclosure.

FIG. 8 is a schematic view of geometric representation of input features in the form of coordinate locations of atom centers, in accordance with some embodiments of the present disclosure.

FIG. 9A illustrates a system for characterizing an interaction between a test compound and a target polymer in accordance with an embodiment of the present disclosure.

FIG. 9B illustrates a system for characterizing an interaction between a test compound and a target polymer in accordance with another embodiment of the present disclosure.

FIG. 9C illustrates a system for characterizing an interaction between a test compound and a target polymer in accordance with another embodiment of the present disclosure, in which the first neural network is a graph based neural network.

FIG. 10 is a system for characterizing an interaction between a test compound and a target polymer, where the characterization is (i) binary-discrete activity and (ii) pKi, and where the system is trained using poses for training compounds in accordance with one embodiment of the present disclosure.

FIG. 11 is a system for characterizing an interaction between a test compound and a target polymer, where the characterization is pKi, and where the pKi is conditioned, in part, on activity, and where the system is trained using poses for training compounds in accordance with one embodiment of the present disclosure.

FIG. 12 is a system for characterizing an interaction between a test compound and a target polymer, where the characterization is activity, and where the activity is conditioned, in part, on both pKi, and a pose quality score, and where the system is trained using poses for training compounds in accordance with one embodiment of the present disclosure.

FIG. 13 is a system for characterizing an interaction between a test compound and a target polymer, where the characterization is activity, and where the activity is conditioned, in part, on both pKi and binding mode score, and where the system is trained using poses for training compounds in accordance with one embodiment of the present disclosure.

FIG. 14 is a system for characterizing an interaction between a test compound and a target polymer, where the characterization is activity and two different compound binding mode scores, and where the system is trained using poses for training compounds in accordance with one embodiment of the present disclosure.

FIG. 15 is a system for characterizing an interaction between a test compound and a target polymer, where the characterization is activity, two different compound binding mode scores and pKi, and where the system is trained using poses for training compounds, in accordance with one embodiment of the present disclosure.

FIG. 16A is a system for characterizing an interaction between a test compound and a target polymer, where the characterization is activity, and where the activity is conditioned, in part, on pKi and a binding mode score, and where the system is trained using poses for training compounds in accordance with one embodiment of the present disclosure.

FIG. 16B is a system for characterizing an interaction between a test compound and a target polymer, where the characterization is activity, and where the activity is conditioned, in part, on pKi and two different binding mode scores, and where the system is trained using poses for training compounds, in accordance with one embodiment of the present disclosure.

FIG. 17 is a depiction of applying multiple function computation elements (g₁, g₂, . . . ) to the voxel inputs (x₁, x₂, . . . , x₁₀₀) and composing the function computation element outputs together using g( ), in accordance with some embodiments of the present disclosure.

FIG. 18 illustrates the insensitivity that machine learning models face when characterizing a pose of a compound with respect to a target polymer in accordance with the prior art.

FIG. 19 illustrates the insensitivity of conventional machine learning models to the quality of the compound-polymer pose, where, as illustrated, the best possible pose receives the same score by a machine learning model as the poor pose, and where an implausible pose receives the same score by the machine learning model as the best possible pose, in accordance with the prior art.

FIG. 20 illustrates an active task conditioned on PoseRanker and Vina scores in accordance with an embodiment of the present disclosure.

FIGS. 21A and 21B provide performance statistics for architectures of the present disclosure (o3-2.8.0 and o4-2.8.0) relative to other architectures (n8b-long and n8b-maxlong).

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The present disclosure provides systems and methods for characterizing an interaction between a compound and a polymer. In the methods a plurality of sets of atomic coordinates are obtained. Each of these sets of atomic coordinates comprises the compound bound to the polymer in a corresponding pose in a plurality of poses. Each respective set of atomic coordinates, or an encoding thereof, is sequentially inputted into a neural network to obtain a corresponding initial embedding as output. In this way a plurality of initial embeddings is calculated. Each initial embedding corresponds to a set of atomic coordinates in the plurality of sets of atomic coordinates. An attention mechanism is applied to the plurality of initial embeddings, in concatenated form, to obtain an attention embedding. In some embodiments the attention mechanism is a neural network that is trained on test data to emphasize some portions of the plurality of initial embeddings while deemphasizing some portions of the plurality of initial embeddings. A pooling function is applied to the attention embedding to derive a pooled embedding. Thus, the pooling function collapses all the initial embeddings representing the plurality of poses into a single composite embedding that represents all the poses in the plurality of poses. The pooled embedding is inputted into a model to obtain an interaction score of the interaction between the compound and the polymer.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

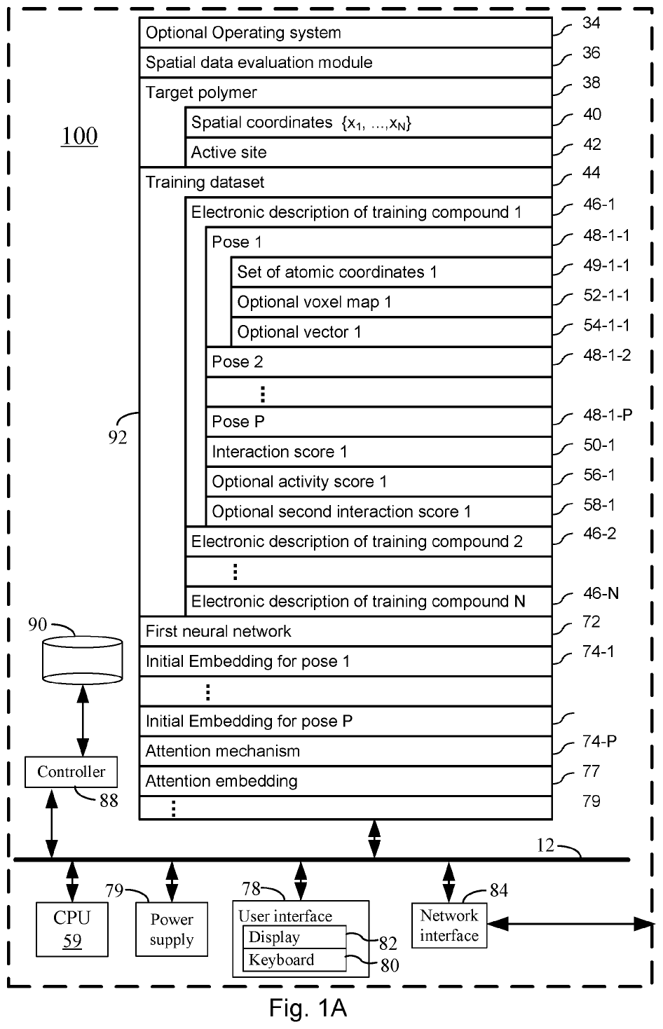

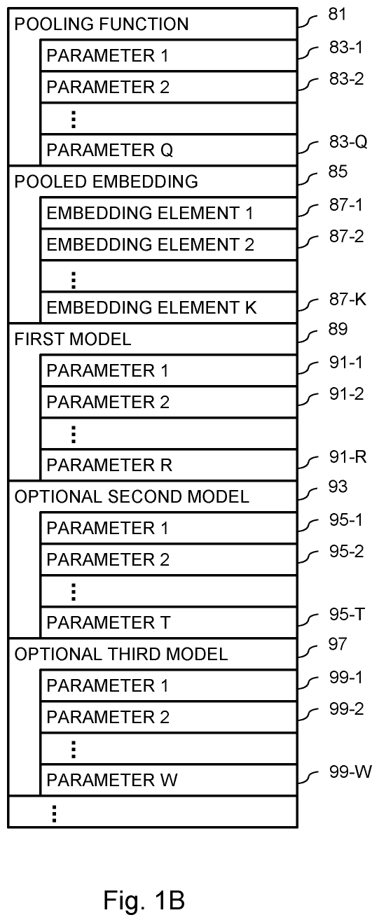

FIG. 1 illustrates a computer system 100 for characterizing an interaction between a test compound and a target polymer. For instance, it can be used as a binding affinity prediction system to generate accurate predictions regarding the binding affinity of one or more test compounds with a target polymer.

Referring to FIG. 1, in typical embodiments, computer system 100 comprises one or more computers. For purposes of illustration in FIG. 1, the computer system 100 is represented as a single computer that includes all of the functionality of the disclosed computer system 100. However, the present disclosure is not so limited. The functionality of the computer system 100 may be spread across any number of networked computers and/or reside on each of several networked computers and/or virtual machines. One of skill in the art will appreciate that a wide array of different computer topologies are possible for the computer system 100 and all such topologies are within the scope of the present disclosure.

Turning to FIG. 1 with the foregoing in mind, the computer system 100 comprises one or more processing units (CPUs) 59, a network or other communications interface 84, a user interface 78 (e.g., including an optional display 82 and optional keyboard 80 or other form of input device), a memory 92 (e.g., random access memory, persistent memory, or combination thereof), one or more magnetic disk storage and/or persistent devices 90 optionally accessed by one or more controllers 88, one or more communication busses 12 for interconnecting the aforementioned components, and a power supply 79 for powering the aforementioned components. To the extent that components of memory 92 are not persistent, data in memory 92 can be seamlessly shared with non-volatile memory 90 or portions of memory 92 that are non-volatile/persistent using known computing techniques such as caching. Memory 92 and/or memory 90 can include mass storage that is remotely located with respect to the central processing unit(s) 59. In other words, some data stored in memory 92 and/or memory 90 may in fact be hosted on computers that are external to computer system 100 but that can be electronically accessed by the computer system 100 over an Internet, intranet, or other form of network or electronic cable using network interface 84. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

The memory 92 of the computer system 100 stores:
an optional operating system 34 that includes procedures for handling various basic system services;
a spatial data evaluation module 36 for characterizing an interaction between a test compound (or training compounds) and a target polymer;
data for a target polymer 38, including structural data (a plurality of atomic spatial coordinates 40 of the target polymer) and optionally active site information 42 of the target polymer;
a training dataset 44 comprising a plurality of electronic descriptions, each electronic description 46 in the plurality of electronic descriptions corresponding to a training compound in a plurality of training compounds (and/or a test compound) and comprising (i) a plurality of poses of the corresponding compound, each respective pose 48 of the corresponding compound represented by (a) a corresponding set of atomic spatial coordinates 49 that detail the atomic coordinates of the corresponding compound in the respective pose with respect to the spatial coordinates 40 of the target polymer 38, (b) an optional corresponding voxel map 52 that details the atomic interactions of the corresponding compound in the respective pose with respect to the target polymer in accordance with the corresponding set of atomic coordinates, and (c) an optional corresponding vector 54 that encodes the interaction between the corresponding compound in the respective pose with respect to the target polymer in accordance with the corresponding set of atomic coordinates 49 and/or the corresponding voxel map 52, (ii) an (first) interaction score 50 between the corresponding compound and the target polymer 38, (iii) an optional activity score 56 between the corresponding compound and the target polymer 38, and (iv) an optional (second) interaction score 58 between the corresponding compound and the target polymer 38;
a first neural network 72 comprising a plurality of parameters, where each respective output of the first neural network provides an initial embedding 74 corresponding to a set of atomic coordinates 49;
an attention mechanism 77 that is collectively applied to the initial embeddings 74 of each pose 48 of a corresponding compound (a particular training or test compound), in concatenated form, to derive an attention embedding 79;
a pooling function 81, having a plurality of parameters 83, where the pooling function is applied to the attention embedding 79 to derive a pooled embedding 85 having a plurality of embedding elements 87;
a first model 89, having a plurality of parameters 91, that is applied to the pooled embedding 85 to (i) obtain a first interaction score of an interaction between the corresponding compound (the particular training or test compound) and the target polymer and/or (ii) condition any other single model and/or group of models;
an optional second model 93, comprising a plurality of parameters 95, where an output of the second model is used to (i) provide a second interaction score of an interaction between the corresponding compound (the particular training or test compound) and the target polymer and/or (ii) condition any other single model and/or group of models;
optionally a third model 97, comprising a third plurality of parameters 99, where an output of the third model is used to (i) provide a third interaction score of an interaction between the corresponding compound (the particular training or test compound) and the target polymer and/or (ii) condition any other single model and/or group of models; and
optionally, any number of additional x^{th} models, each such additional x^{th} model comprising a corresponding plurality of parameters, where an output of the additional x^{th} model is used, at least in part, to (i) provide a characterization of the interaction between the corresponding compound (the particular training or test compound) and the target polymer and/or (ii) condition any other single model and/or group of models.

In some implementations, one or more of the above identified data elements or modules of the computer system 100 are stored in one or more of the previously mentioned memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 92 and/or 90 (and optionally 52) optionally stores a subset of the modules and data structures identified above. Furthermore, in some embodiments the memory 92 and/or 90 (and optionally 52) stores additional modules and data structures not described above. In some embodiments, the first neural network 72 is replaced with another form of model.

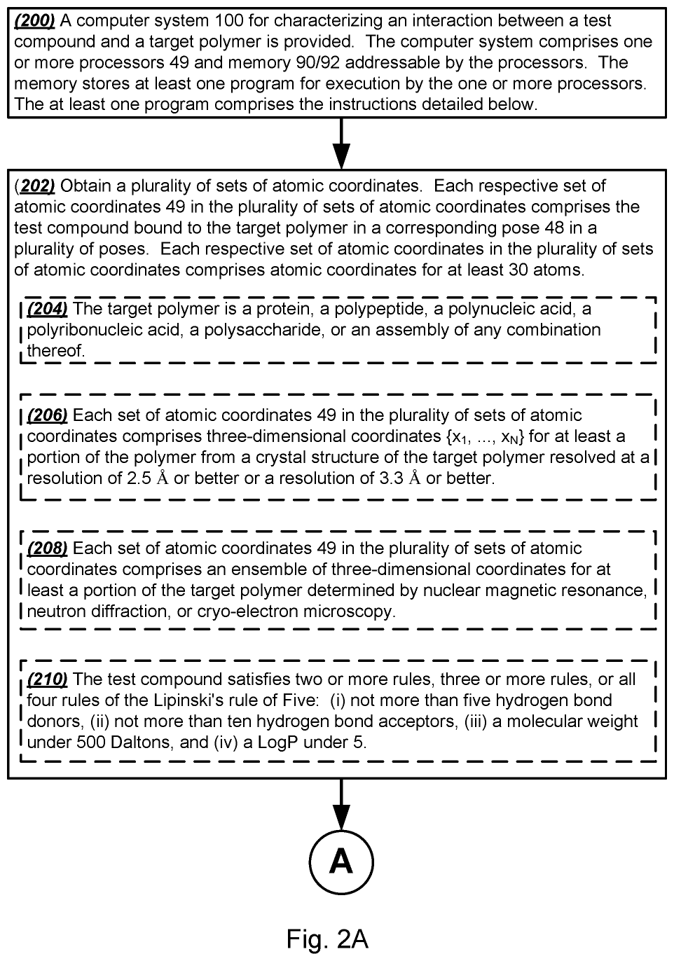

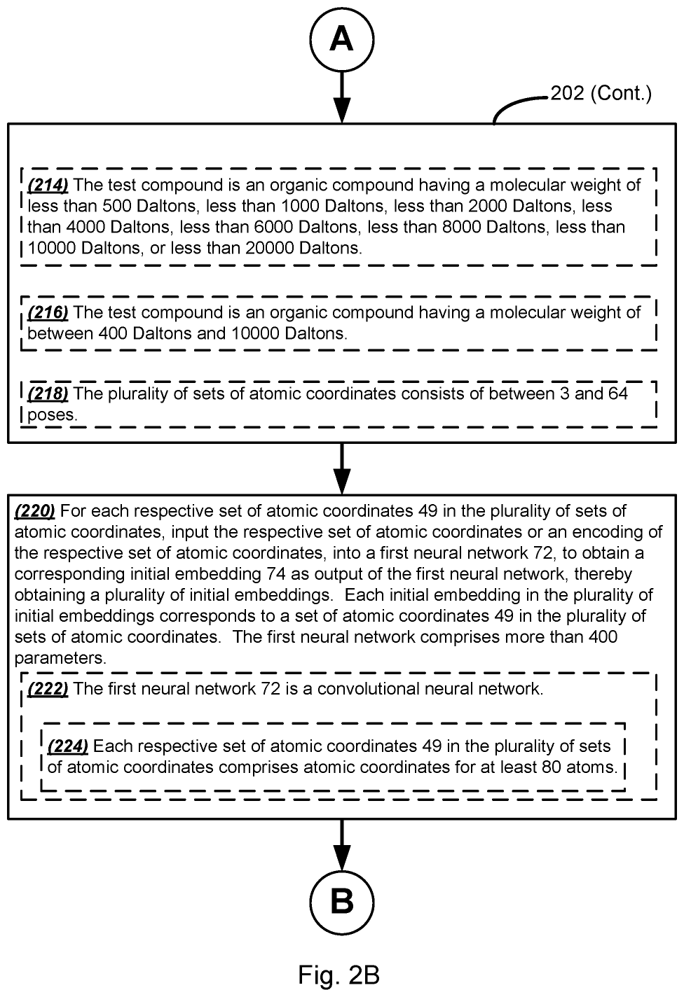

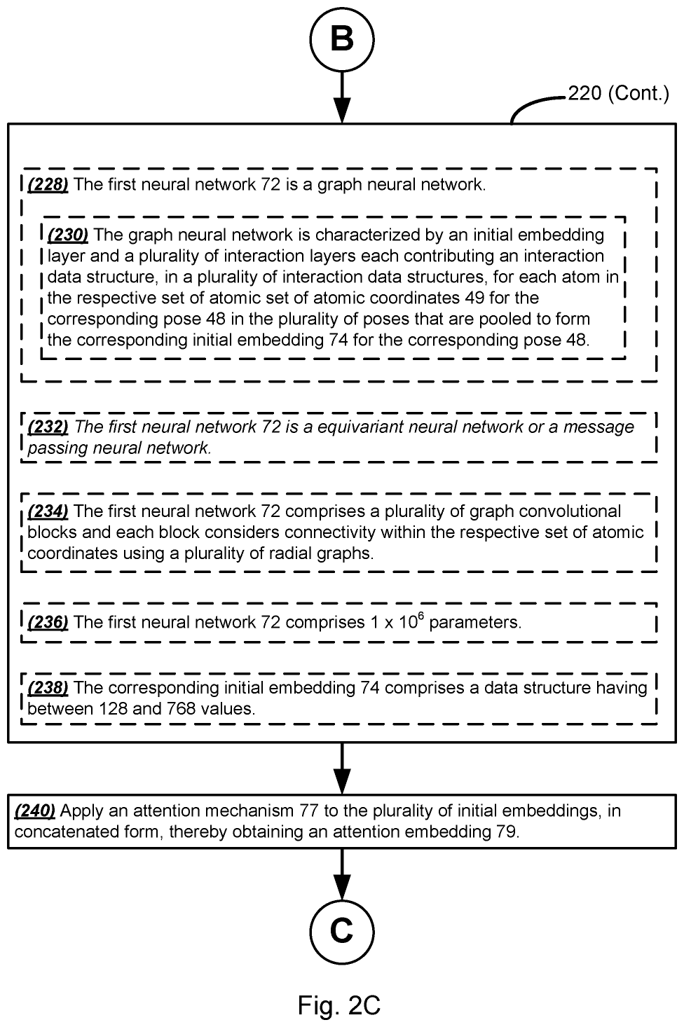

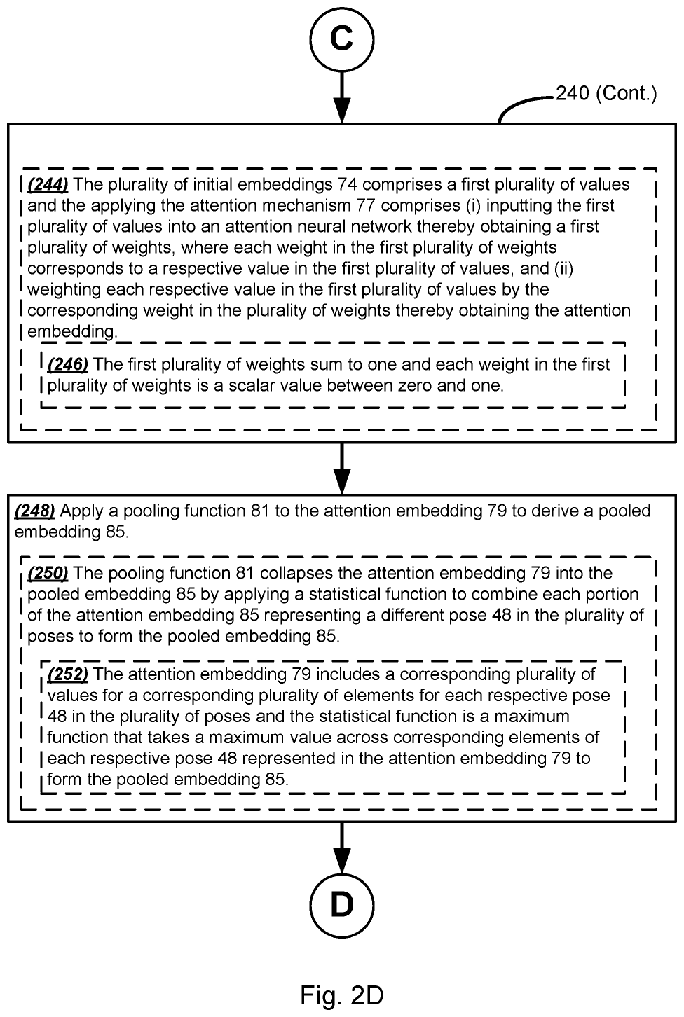

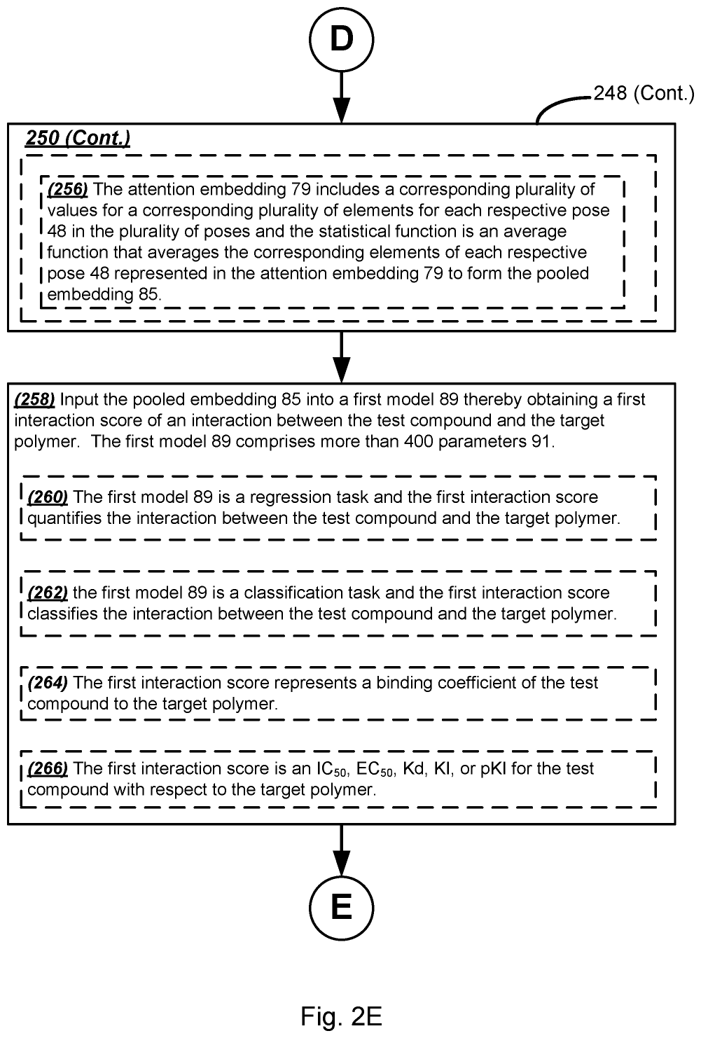

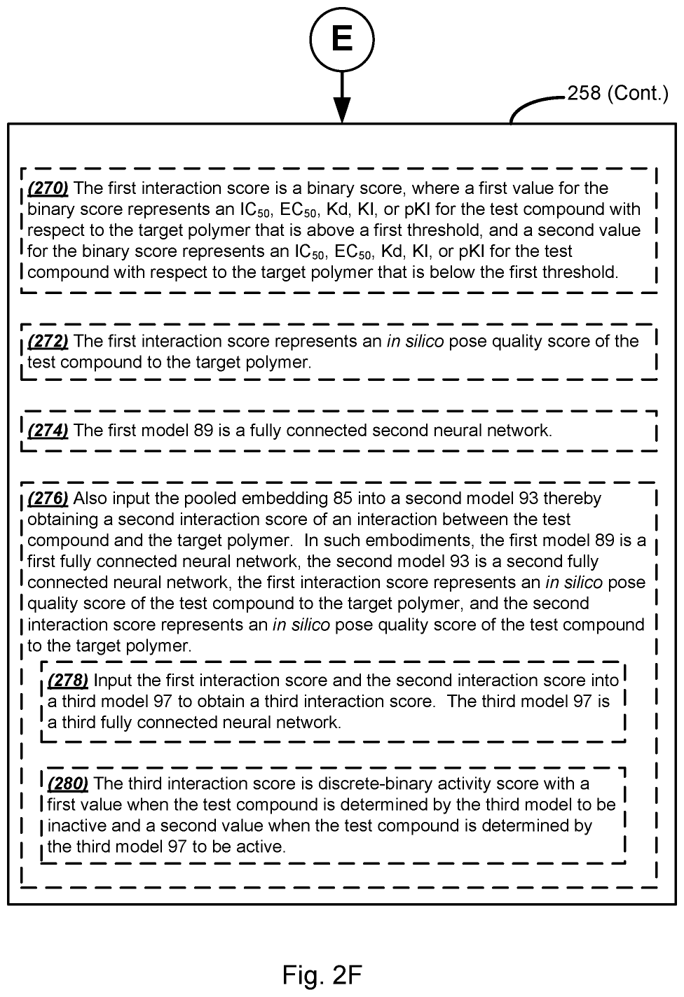

Now that a system for characterizing an interaction between a test compound and a target polymer has been disclosed, methods for performing such characterization is detailed with reference to FIG. 2 and discussed below.

Block 200. Referring to block 200 of FIG. 2A, a computer system 100 for characterizing an interaction between a test compound and a target polymer is provided. As discussed above in conjunction with FIG. 1, the computer system 100 comprises one or more processors 59 and memory 90/92 addressable by the one or more processors. The memory stores at least one program for execution by the one or more processors. The at least one program comprises instructions detailed below.

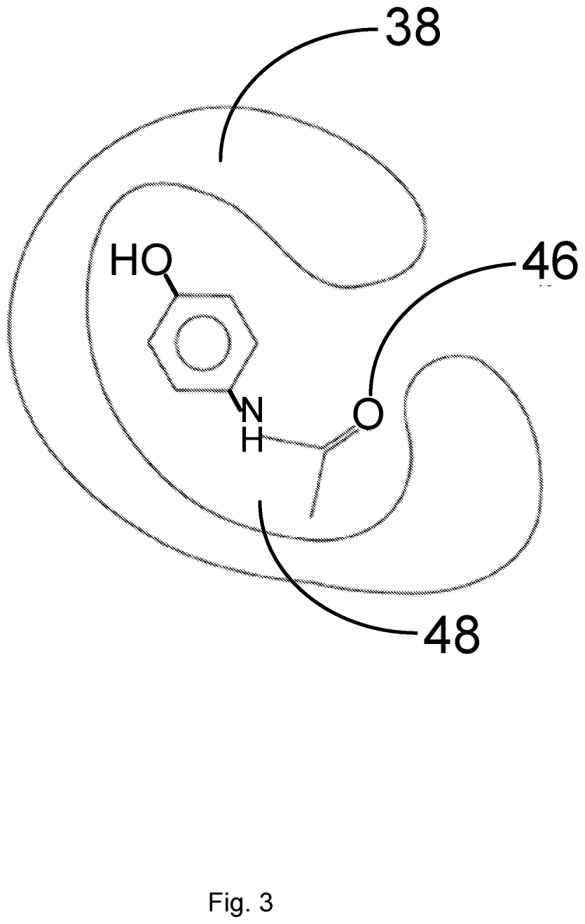

Blocks 202 through 218. Referring to block 202 of FIG. 2A, a plurality of sets of atomic coordinates is obtained. Each respective set of atomic coordinates 49 in the plurality of sets of atomic coordinates comprises the test compound bound to the target polymer in a corresponding pose 48 in a plurality of poses. In other words, with reference to FIG. 1A, each respective set of atomic coordinates 49 includes both the atomic coordinates of the test compound and at least a subset of the spatial coordinates of the target polymer that is considered by the first neural network. For instance in some embodiments, the set of atomic coordinates 49 consists of the atomic coordinates of the test compound and the atomic coordinates of the portion of the target polymer that make up an active site to which the test compound has been docked. In some embodiments the target polymer comprises multiple active sites, and the test compound has been docked to one of the active sites. FIG. 3 illustrates a pose 48 of a test compound in an active site of a target polymer 38. Each respective set of atomic coordinates in the plurality of sets of atomic coordinates comprises atomic coordinates for at least 25, 30, 35, 40, 45, 50, 55, 60, 65, 70, 75, 80, 85, 90, 95, 100, 150, 200, 250, or 300 atoms. In some embodiments, a set of atomic coordinates in the plurality of sets of atomic coordinates comprises atomic coordinates for at least 400 atoms of the target polymer in addition to the atomic coordinates of the test compound. In some embodiments, a set of atomic coordinates in the plurality of sets of atomic coordinates comprises atomic coordinates for at least 25 atoms, at least 50 atoms, at least 100 atoms, at least 200 atoms, at least 300 atoms, at least 400 atoms, at least 1000 atoms, at least 2000 atoms, or at least 5000 atoms of the target polymer in addition to the atomic coordinates of the test compound. In some embodiments, only the coordinates of the active site of the target polymer 38 where ligands are expected to bind the target polymer is present in each respective set of atomic coordinates in the plurality of sets of atomic coordinates in addition to the coordinates of the corresponding test compounds.

Referring to block 204 of FIG. 2A, in some embodiments, the target polymer is a protein, a polypeptide, a polynucleic acid, a polyribonucleic acid, a polysaccharide, or an assembly of any combination thereof.

In some embodiments, a target polymer 38 is a large molecule composed of repeating residues. In some embodiments, the target polymer 38 is a natural material. In some embodiments, the target polymer 38 is a synthetic material. In some embodiments, the target polymer 38 is an elastomer, shellac, amber, natural or synthetic rubber, cellulose, Bakelite, nylon, polystyrene, polyethylene, polypropylene, polyacrylonitrile, polyethylene glycol, or a polysaccharide.

In some embodiments, the target polymer 38 is a heteropolymer (copolymer). A copolymer is a polymer derived from two (or more) monomeric species, as opposed to a homopolymer where only one monomer is used. Copolymerization refers to methods used to chemically synthesize a copolymer. Examples of copolymers include, but are not limited to, ABS plastic, SBR, nitrile rubber, styrene-acrylonitrile, styrene-isoprene-styrene (SIS) and ethylene-vinyl acetate. Since a copolymer comprises at least two types of constituent units (also structural units, or particles), copolymers can be classified based on how these units are arranged along the chain. These include alternating copolymers with regular alternating A and B units. See, for example, Jenkins, 1996, “Glossary of Basic Terms in Polymer Science,” Pure Appl. Chem. 68 (12): 2287-2311, which is hereby incorporated herein by reference in its entirety. Additional examples of copolymers are periodic copolymers with A and B units arranged in a repeating sequence (e.g. (A-B-A-B-B-A-A-A-A-B-B-B)ₙ). Additional examples of copolymers are statistical copolymers in which the sequence of monomer residues in the copolymer follows a statistical rule. See, for example, Painter, 1997, Fundamentals of Polymer Science, CRC Press, 1997, p 14, which is hereby incorporated by reference herein in its entirety. Still other examples of copolymers that may be evaluated using the disclosed systems and methods are block copolymers comprising two or more homopolymer subunits linked by covalent bonds. The union of the homopolymer subunits may require an intermediate non-repeating subunit, known as a junction block. Block copolymers with two or three distinct blocks are called diblock copolymers and triblock copolymers, respectively.

In some embodiments, the target polymer 38 comprises 50 or more, 100 or more, 150 or more, 200 or more, 300 or more, 400 or more, 500 or more, 600 or more, 700 or more, 800 or more, 900 or more, or 1000 or more atoms.

In some embodiments, the target polymer 38 is in fact a plurality of polymers (e.g., 2 or more, 3, or more, 10 or more, 100 or more, 1000 or more, or 5000 or more polymers), where the respective polymers in the plurality of polymers do not all have the same molecular weight. In some such embodiments, the target polymers 38 in the plurality of polymers share at least 50 percent, at least 60 percent, at least 70 percent, at least 80 percent, or at least 90 percent sequence identity and fall into a weight range with a corresponding distribution of chain lengths. In some embodiments, the target polymer 38 is a branched polymer molecule comprising a main chain with one or more substituent side chains or branches. Types of branched polymers include, but are not limited to, star polymers, comb polymers, brush polymers, dendronized polymers, ladders, and dendrimers. See, for example, Rubinstein et al., 2003, Polymer physics, Oxford; New York: Oxford University Press. p. 6, which is hereby incorporated by reference herein in its entirety.

In some embodiments, the target polymer is a polypeptide. As used herein, the term “polypeptide” means two or more amino acids or residues linked by a peptide bond. The terms “polypeptide” and “protein” are used interchangeably herein and include oligopeptides and peptides. An “amino acid,” “residue” or “peptide” refers to any of the twenty standard structural units of proteins as known in the art, which include imino acids, such as proline and hydroxyproline. The designation of an amino acid isomer may include D, L, R and S. The definition of amino acid includes nonnatural amino acids. Thus, selenocysteine, pyrrolysine, lanthionine, 2-aminoisobutyric acid, gamma-aminobutyric acid, dehydroalanine, ornithine, citrulline and homocysteine, as nonlimiting examples, are all considered amino acids. Other variants or analogs of the amino acids are known in the art. Thus, a polypeptide may include synthetic peptidomimetic structures such as peptoids. See Simon et al., 1992, Proceedings of the National Academy of Sciences USA, 89, 9367, which is hereby incorporated by reference herein in its entirety. See also Chin et al., 2003, Science 301, 964; and Chin et al., 2003, Chemistry & Biology 10, 511, each of which is incorporated by reference herein in its entirety.

The target polymer 38 evaluated in accordance with some embodiments of the disclosed systems and methods may also have any number of posttranslational modifications. Thus, a target polymer 38 includes those polymers that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphotases and kinases). Other types of posttranslational modifications are known in the art and are within the scope of the target polymers 38 of the present disclosure.

In some embodiments, the target polymer 38 is a surfactant. Surfactants are compounds that lower the surface tension of a liquid, the interfacial tension between two liquids, or that between a liquid and a solid. Surfactants may act as detergents, wetting agents, emulsifiers, foaming agents, and dispersants. Surfactants are usually organic compounds that are amphiphilic, meaning they contain both hydrophobic groups (their tails) and hydrophilic groups (their heads). Therefore, a surfactant molecule contains both a water insoluble (or oil soluble) component and a water soluble component. Surfactant molecules will diffuse in water and adsorb at interfaces between air and water or at the interface between oil and water, in the case where water is mixed with oil. The insoluble hydrophobic group may extend out of the bulk water phase, into the air or into the oil phase, while the water soluble head group remains in the water phase. This alignment of surfactant molecules at the surface modifies the surface properties of water at the water/air or water/oil interface.

Examples of ionic surfactants include ionic surfactants such as anionic, cationic, or zwitterionic (ampoteric) surfactants. In some embodiments, the target object 58 is a reverse micelle or liposome.

In some embodiments, the target polymer 38 is a fullerene. A fullerene is any molecule composed entirely of carbon, in the form of a hollow sphere, ellipsoid or tube. Spherical fullerenes are also called buckyballs, and they resemble the balls used in association football. Cylindrical ones are called carbon nanotubes or buckytubes. Fullerenes are similar in structure to graphite, which is composed of stacked graphene sheets of linked hexagonal rings; but they may also contain pentagonal (or sometimes heptagonal) rings.

Referring to block 206 of FIG. 2A, in some embodiments, each set of atomic coordinates 49 in the plurality of sets of atomic coordinates comprises three-dimensional coordinates {x₁, . . . , x_{N}} for at least a portion of the polymer from a crystal structure of the target polymer resolved at a resolution (e.g., by X-ray crystallographic techniques) of 3.3 Å or better, 3.2 Å or better, 3.1 Å or better, 3.0 Å or better, 2.5 Å or better, 2.2 Å or better, 2.0 Å or better, 1.9 Å or better, 1.85 Å or better, 1.80 Å or better, 1.75 Å or better, or 1.70 Å or better. In some embodiments the portion of the polymer from a crystal structure of the target polymer consists of atomic coordinates for less than 25, 30, 35, 40, 45, 50, 55, 60, 65, 70, 75, 80, 85, 90, 95, 100, 150, 200, 250, or 300 atoms. In some embodiments, the portion of the polymer from a crystal structure of the target polymer consists of atomic coordinates for less than 400 atoms of the target polymer in addition to the atomic coordinates of the test compound. In some embodiments, the portion of the polymer from a crystal structure of the target polymer consists of atomic coordinates for less than 25 atoms, less than 50 atoms, less than 100 atoms, less than 200 atoms, less than 300 atoms, less than 400 atoms, less than 1000 atoms, less than 2000 atoms, or less than 5000 atoms of the target polymer.

In some embodiments each set of atomic coordinates 49 in the plurality of sets of atomic coordinates comprises three-dimensional coordinates {x₁, . . . , x_{N}} for at least a portion of the polymer from a structure prediction program such as AlphaFold2. Jumper et al., 2021, “Highly accurate protein structure prediction with AlphaFold,” Nature 596, pp. 583-589, which is hereby incorporated by reference.

Referring to block 208 of FIG. 2A, in some embodiments each set of atomic coordinates 49 in the plurality of sets of atomic coordinates comprises an ensemble of three-dimensional coordinates for at least a portion of the target polymer determined by nuclear magnetic resonance, neutron diffraction, or cryo-electron microscopy. In some embodiments this portion of the polymer consists of atomic coordinates for less than 25, 30, 35, 40, 45, 50, 55, 60, 65, 70, 75, 80, 85, 90, 95, 100, 150, 200, 250, or 300 atoms. In some embodiments, this portion of the polymer consists of atomic coordinates for less than 400 atoms of the target polymer in addition to the atomic coordinates of the test compound. In some embodiments, the portion of the polymer consists of atomic coordinates for less than 25 atoms, less than 50 atoms, less than 100 atoms, less than 200 atoms, less than 300 atoms, less than 400 atoms, less than 1000 atoms, less than 2000 atoms, or less than 5000 atoms of the target polymer. In some embodiments, the ensemble of three-dimensional coordinates comprises ten or more, twenty or more or thirty or more atomic structures of the at least a portion of the target polymer having a backbone RMSD of 1.0 Å or better, 0.9 Å or better, 0.8 Å or better, 0.7 Å or better, 0.6 Å or better, 0.5 Å or better, 0.4 Å or better, 0.3 Å or better, or 0.2 Å or better.

In some embodiments, the target polymer 38 includes two different types of polymers, such as a nucleic acid bound to a polypeptide. In some embodiments, the native target polymer includes two polypeptides bound to each other. In some embodiments, the native target polymer under study includes one or more metal ions (e.g. a metalloproteinase with one or more zinc atoms). In such instances, the metal ions and or the organic small molecules may be included in the atomic coordinates 40 for the target polymer.

In some embodiments the target polymer 38 is a polymer and there are ten or more, twenty or more, thirty or more, fifty or more, one hundred or more, between one hundred and one thousand, or less than 500 residues in the target polymer.

In some embodiments, the atomic coordinates of the target polymer 38 are determined using modeling methods such as ab initio methods, density functional methods, semi-empirical and empirical methods, molecular mechanics, chemical dynamics, or molecular dynamics.

In some embodiments, each respective set of atomic coordinates 49 is represented by the Cartesian coordinates of the centers of the atoms comprising the target polymer 38. In some alternative embodiments, each respective set of atomic coordinates 49 are represented by the electron density of the target polymer as measured, for example, by X-ray crystallography. For example, in some embodiments, the atomic coordinates 40 comprise a 2F_{observed}−F_{calculated} electron density map computed using the calculated atomic coordinates of the target polymer 38, where F_{observed} is the observed structure factor amplitudes of the target polymer and F_{calculated} is the structure factor amplitudes calculated from the calculated atomic coordinates of the target polymer 38.

In various other embodiments, each respective set of atomic coordinates 49 is obtained from any of a variety of sources including, but not limited to, structure ensembles generated by solution NMR, co-complexes as interpreted from X-ray crystallography, neutron diffraction, cryo-electron microscopy, sampling from computational simulations, homology modeling, rotamer library sampling, or any combination thereof.

Referring to block 210 of FIG. 2A, in some embodiments, the test compound satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, the test compound satisfies one or more criteria in addition to Lipinski's Rule of Five. For example, in some embodiments, the test compound has five or fewer aromatic rings, four or fewer aromatic rings, three or fewer aromatic rings, or two or fewer aromatic rings.

Referring to block 214 of FIG. 2A, in some embodiments, the test compound is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons. However, some embodiments of the disclosed systems and methods have no limitation on the size of the test compound. For instance, in some embodiments, the test compound is itself a large polymer, such as an antibody.

Referring to block 216 of FIG. 2A, in some embodiments the test compound is an organic compound having a molecular weight of between 400 Daltons and 10000 Daltons.

Referring to block 218 of FIG. 2A, in some embodiments the plurality of sets of atomic coordinates consists of between 3 and 64 poses. In some embodiments, the target polymer 38 is a polymer with an active site, and each of the poses is obtained by docking the test compound into the active site of the target polymer. In some embodiments, the test compound is docked onto the target polymer 38 a plurality of times to form a plurality of poses. In some embodiments, the test compound is docked onto the target polymer 38 twice, three times, four times, five or more times, ten or more times, fifty or more times, 100 or more times, or a 1000 or more times. Each such docking represents a different pose of the test compound docked onto the target polymer 38. In some embodiments, the target polymer 38 is a polymer with an active site and the test compound is docked into the active site in each of plurality of different ways, each such way representing a different pose. In some embodiments, the target polymer comprises a plurality of active sites and the test compound is docked into one of the active sites in each of plurality of different ways, each such way representing a different pose. In some such embodiments, separate studies are individually also conducted on one or more of the other active sites of the target polymer using the systems and methods of the present disclosure.

In some embodiments, each pose of a test compound is determined by AutoDock Vina. See, Trott and Olson, “AutoDock Vina: improving the speed and accuracy of docking with a new scoring function, efficient optimization and multithreading,” Journal of Computational Chemistry 31 (2010) 455-461. In some embodiments, one docking program is used to determine some of the poses for a test compound and another docking program is used to determine other poses for the test compound. In some embodiments, Quick Vina 2 (Alhossary et al., 2015, “Fast, accurate, and reliable molecular docking with QuickVina,” Bioinformatics 31:13, pp. 2214-2216), VinaLC (Zhang et al., 2013, “Message Passing Interface and Multithreading Hybrid for Parallel Molecular Docking of Large Databases on Petascale High Performance Computing Machines,” J. Comput. Chem. DOI: 10.1002/jcc.23214), Smina (Koes et al, 2013, “Lessons learned in empirical scoring with smina from the CSAR 2011 benchmarking exercise,” Journal of chemical information and modeling 53:8, pp. 1893-1904), or CUina (Morrison et al., “Efficient GPU Implementation of AutoDock Vina,” COMP poster 3432389), each of which is hereby incorporated by reference, is used to determine poses for the test compound.

In some embodiments, the plurality of sets of atomic coordinates is an ensemble from an ensembled docking algorithm such as disclosed in Stafford et al., 2022, “AtomNet PoseRanker: Enriching Ligand Pose Quality for Dynamic Proteins in Virtual High-Throughput Screens,” Journal of Chemical Information and Modeling 62, pp. 1178-1189, which is hereby incorporated by reference. In some such embodiments the ensemble consists of between 3 and 64, between 4 and 128, between 5 and 32, more than 5, or between 8 and 25 structurally similar poses.

In some embodiments, each pose of the docked test compound is scored against several different conformations (e.g., between 2 and 100) of the target protein. In some embodiments, each pose (for instance in an ensemble of poses) is scored against a fixed conformation of the target protein.

In some embodiments, the test compound is docked to the target polymer 38 by either random pose generation techniques, or by biased pose generation. In some embodiments, the test compound is docked to the target polymer 38 by Markov chain Monte Carlo sampling. In some embodiments, such sampling allows the full flexibility of the test compound in the docking calculations and a scoring function that is the sum of the interaction energy between the test compound and the target polymer 38 as well as the conformational energy of the test compound. See, for example, Liu and Wang, 1999, “MCDOCK: A Monte Carlo simulation approach to the molecular docking problem,” Journal of Computer-Aided Molecular Design 13, 435-451, which is hereby incorporated by reference. In some such embodiments, the poses represented by the plurality of sets of atomic coordinates are the poses that receive a top score relative to all other poses tested (e.g., the top 256 scores and thus 256 poses, the top 128 scores and thus 128 poses, the top 64 scores and thus 64 poses, the top 32 scores and thus 32 poses, etc.).

In some embodiments, algorithms such as DOCK (Shoichet, Bodian, and Kuntz, 1992, “Molecular docking using shape descriptors,” Journal of Computational Chemistry 13(3), pp. 380-397; and Knegtel et al., 1997 “Molecular docking to ensembles of protein structures,” Journal of Molecular Biology 266, pp. 424-440, each of which is hereby incorporated by reference) are used to find a plurality of poses for the test compound against the target polymer 38. Such algorithms model the target polymer 38 and the test compound as rigid bodies. The docked conformation is searched using surface complementary to find poses 48.

In some embodiments, algorithms such as AutoDOCK (Morris et al., 2009, “AutoDock4 and AutoDockTools4: Automated Docking with Selective Receptor Flexibility,” J. Comput. Chem. 30(16), pp. 2785-2791; Sotriffer et al., 2000, “Automated docking of ligands to antibodies: methods and applications,” Methods: A Companion to Methods in Enzymology 20, pp. 280-291; and “Morris et al., 1998, “Automated Docking Using a Lamarckian Genetic Algorithm and Empirical Binding Free Energy Function,” Journal of Computational Chemistry 19: pp. 1639-1662, each of which is hereby incorporated by reference) are used to find a plurality of poses for the test compound against the target polymer 38. AutoDOCK uses a kinematic model of the ligand and supports Monte Carlo, simulated annealing, the Lamarckian Genetic Algorithm, and Genetic algorithms. Accordingly, in some embodiments the plurality of different poses for the test compound are obtained by Markov chain Monte Carlo sampling, simulated annealing, Lamarckian Genetic Algorithms, or genetic algorithms, using a docking scoring function.

In some embodiments, algorithms such as FlexX (Rarey et al., 1996, “A Fast Flexible Docking Method Using an Incremental Construction Algorithm,” Journal of Molecular Biology 261, pp. 470-489, which is hereby incorporated by reference) are used to find a plurality of poses for the test compound against the target polymer. FlexX does an incremental construction of the test compound at the active site of the target polymer 38 using a greedy algorithm. Accordingly, in some embodiments, the plurality of different poses for the test compound are obtained by a greedy algorithm.

In some embodiments, algorithms such as GOLD (Jones et al., 1997, “Development and Validation of a Genetic Algorithm for flexible Docking,” Journal Molecular Biology 267, pp. 727-748, which is hereby incorporated by reference) are used to find a plurality of poses for the test compound against the target polymer 38. GOLD stands for Genetic Optimization for Ligand Docking. GOLD builds a genetically optimized hydrogen bonding network between the test compound and the target polymer 38.

In some embodiments, molecular dynamics is performed on the target polymer (or a portion thereof such as the active site of the target polymer) and the test compound to identify the plurality of poses. During the molecular dynamics run, the atoms of the target polymer and the test compound are allowed to interact for a fixed period of time, giving a view of the dynamical evolution of the system. The trajectory of atoms in the target polymer and the test compound are determined by numerically solving Newton's equations of motion for a system of interacting particles, where forces between the particles and their potential energies are calculated using interatomic potentials or molecular mechanics force fields. See Alder and Wainwright, 1959, “Studies in Molecular Dynamics. I. General Method,” J. Chem. Phys. 31 (2): 459; and Bibcode, 1959, J. Ch. Ph. 31, 459A, doi:10.1063/1.1730376, each of which is hereby incorporated by reference. Thus, in this way, the molecular dynamics run produces a trajectory of the target polymer and the respective test compound over time. This trajectory comprises the trajectory of the atoms in the target polymer and the test compound. In some embodiments, a subset of the plurality of different poses is obtained by taking snapshots of this trajectory over a period of time. In some embodiments, poses are obtained from snapshots of several different trajectories, where each trajectory comprises a different molecular dynamics run of the target polymer interacting with the test compound. In some embodiments, prior to a molecular dynamics run, the test compound is first docked into an active site of the target polymer using a docking technique.

Blocks 220 through 238. Referring to block 220 of FIG. 2B, for each respective set of atomic coordinates 49 in the plurality of sets of atomic coordinates, the respective set of atomic coordinates, or an encoding of the respective set of atomic coordinates, is inputted into a first neural network 72 to obtain a corresponding initial embedding 74, as output of the first neural network, thereby obtaining a plurality of initial embeddings 74-1, . . . , 74-N. Each initial embedding 74 in the plurality of initial embeddings 74-1, . . . , 74-N corresponds to a set of atomic coordinates 49 in the plurality of sets of atomic coordinates 49-1-1, . . . , 49-1-N. In some embodiments, the first neural network 72 comprises more than 400 parameters. In some embodiments, the respective set of atomic coordinates, or an encoding of the respective set of atomic coordinates, that is inputted into the first neural network 72 consists of between 20 bits and 20000 bits of information. In some embodiments, the respective set of atomic coordinates, or an encoding of the respective set of atomic coordinates, that is inputted into the first neural network 72 comprises 20 bits, 40 bits, 60 bits, 80 bits, 100 bits, 200 bits, 300 bits, 400 bits, 500 bits, 600 bits, 700 bits, 800 bits, 900 bits, or 1000 bits of information. In some embodiments, the respective set of atomic coordinates, or an encoding of the respective set of atomic coordinates, that is inputted into the first neural network 72 comprises 2000 bits, 4000 bits, 6000 bits, 8000 bits, or 10,000 bits of information. In some embodiments, the first neural network 72 comprises more than 400 parameters, more than 1000 parameters, more than 2000 parameters, more than 5000 parameters, more than 10,000 parameters, more than 100,000 parameters, or more than 1×10⁶ parameters. In some embodiments, the amount of information in the respective set of atomic coordinates inputted into the first neural network coupled with the number of the parameters of the neural network results in the performance of more than 10,000 computations, more than 100,000 computations, more than 1×10⁶ computations, more than 5×10⁶ computations, or more than 1×10⁷ computations to calculate the initial embedding 74 using the first neural network 74.

Referring to block 222 of FIG. 2B, in some embodiments, the first neural network 72 is a convolutional neural network. Referring to block 224 of FIG. 2B, in some such embodiments, each respective set of atomic coordinates 49 in the plurality of sets of atomic coordinates comprises atomic coordinates for at least 80 atoms (e.g., the atoms of the target compound in addition to those atoms of the target protein that will be considered by the first neural network).

In some embodiments the respective set of atomic coordinates 49 is converted into a corresponding voxel map 52. As such, the corresponding voxel map 52 represents the test compound with respect to the target polymer 38 in a corresponding pose 48. In some such embodiments, the voxel map 52 is unfolded into a corresponding vector 54 and inputted into the first neural network 72. In some such embodiments, the first neural network 72 is a convolutional neural network that, in turn, provides the corresponding initial embedding 74 for the corresponding pose 48 as output. In some such embodiments, the corresponding vector 54 referenced above is a one-dimensional vector. In some embodiments, the corresponding vector 54 comprises 10 or more elements, 20 or more elements, 100 or more elements, 500 or more elements, 1000 or more elements, or 10,000 or more elements. In some such embodiments each such element is represented by a different bit in a data structure inputted into the first neural network.

In some embodiments, the first neural network 72 is any of the convolutional neural networks disclosed in Wallach et al., 2015, “AtomNet: A Deep Convolutional Neural Network for Bioactivity Prediction in Structure-based Drug Discovery,” arXiv:1510.02855v1, or U.S. Pat. Nos. 11,080,570; 10,546,237; 10,482,355; 10,002,312; or 9,373,059, each of which is hereby incorporated by reference. More details on obtaining the corresponding initial embedding 74 for the corresponding pose 48 of the test compound with respect to the target polymer 38 using a convolutional neural network as the first neural network 72 are disclosed below in the section entitled “Using a convolutional neural network as the first neural network 72.”

In some embodiments, the first neural network 72 is an equivariant neural network. Nonlimiting examples of the equivariant convolutional neural network are disclosed in Thomas et al., 2018, “Tensor field networks: Rotation- and translation-equivariant neural networks for 3D point clouds,” arXiv: 1802.08219; Anderson et al., 2019, “Cormorant: Covariant Molecular Neural Networks,” Neural Information Processing Systems; Johannes et al., 2020, “Directional Message Passing For Molecular Graphs,” International Conference on Learning Representations; Townshend et al., 2021, “ATOM3D: Tasks On Molecules in Three Dimensions,” International Conference on Learning Representations; Jing et al., 2009, “Learning from Protein Structure with Geometric Vector Perceptrons,” arXiv: 2009.01411; and Satorras et al., 2021, “E(n) Equivariant Graph Neural Networks,” arXiv: 2102.09844, each of which is hereby incorporated by reference.

Referring to block 228 of FIG. 2C, in some embodiments, the first neural network 72 is a graph neural network. Referring to block 230 of FIG. 2C, in some such embodiments, the graph neural network is characterized by an initial embedding layer and a plurality of interaction layers that each contribute an interaction data structure, in a plurality of interaction data structures, for each atom in the respective set of atomic set of atomic coordinates 49 for the corresponding pose 48 in the plurality of poses that are pooled to form the corresponding initial embedding 74 for the corresponding pose 48.

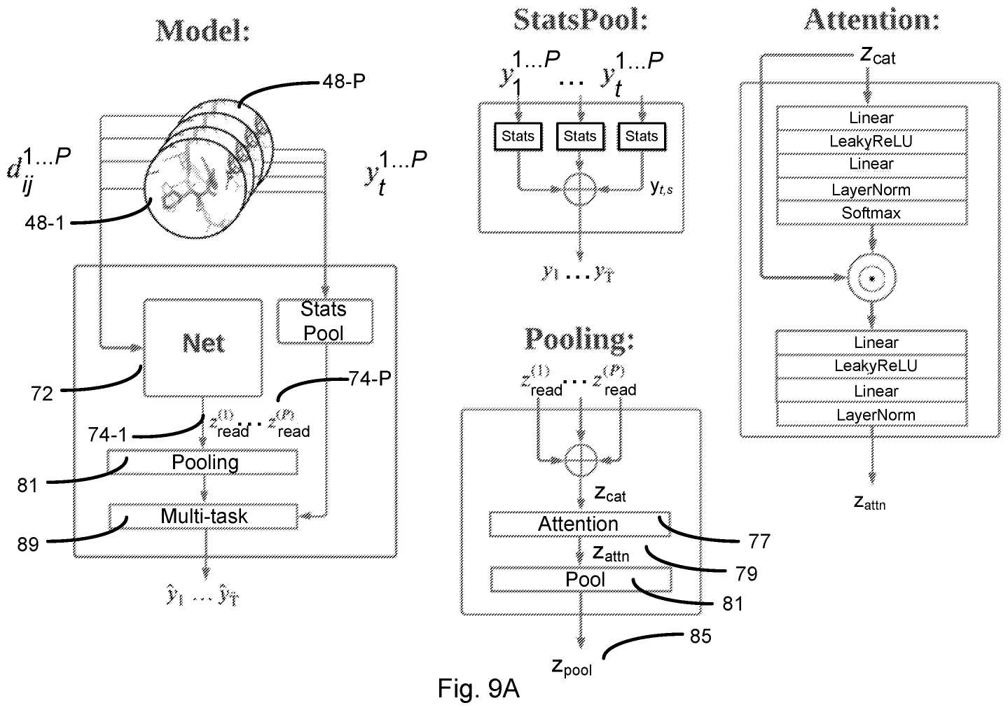

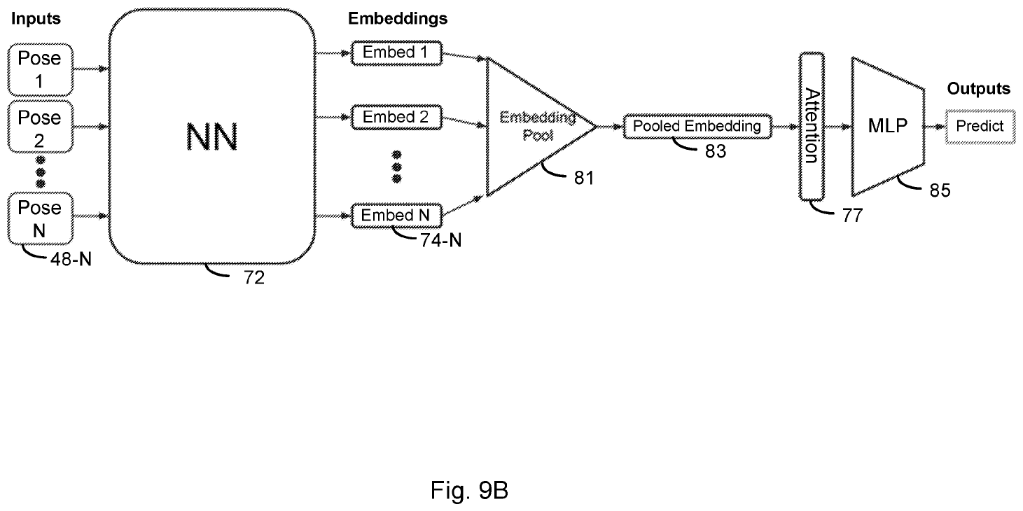

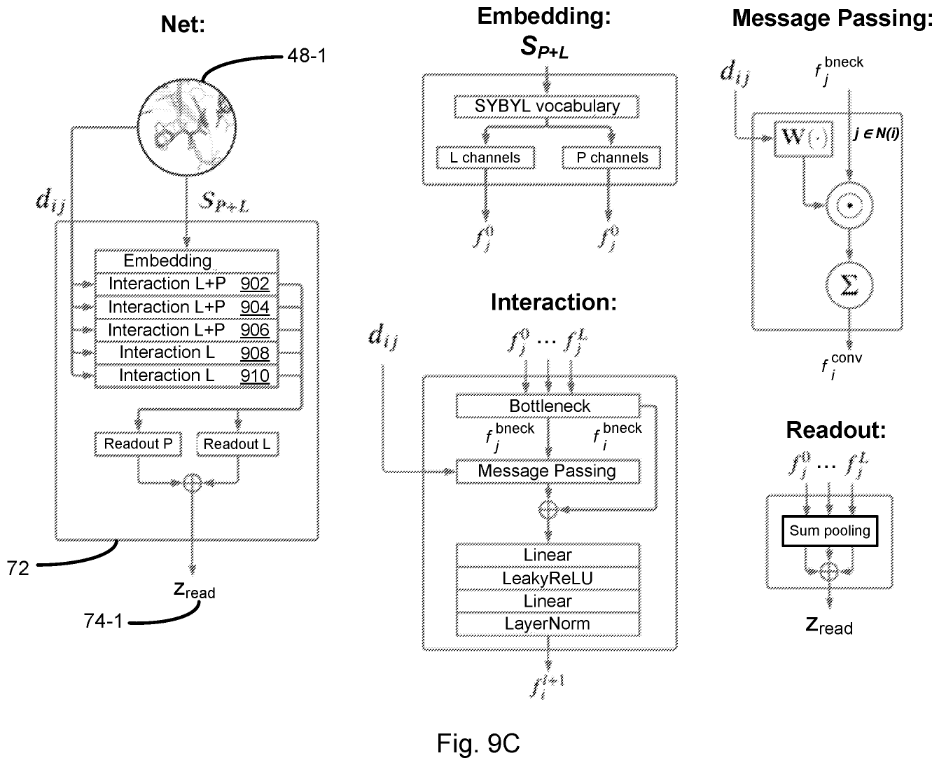

FIG. 9B illustrates an embodiment of the present disclosure in which the first neural network 72 is a graph neural network (GCN). In some embodiments, the GCN takes the three dimensional coordinates of the protein-ligand pose 48, along with a one-hot atom encoding that simultaneously identifies element type, target protein/compound, and hybridization state. In some embodiments, connectivity is defined purely by radial functions without use of chemical bonds. In some embodiments, a radial cutoff

$${R}_{c}^{l}$$

is used at layer l to define a radial graph, with the neighborhood for atom i defined as [symbol] (i)={j:dᵢⱼ<R_{c}}, where dᵢⱼ=∥rᵢ−rⱼ∥ is the pairwise distance between atoms i and j. At each layer l there is a feature vector

$${f}_{i}^{l} ∈ {ℛ}^{{N}_{f}^{l}}$$

at atom i∈ [symbol] _{P}/_{L}/_{P+L}, where

$${N}_{f}^{l}$$

is the number of features at layer l, and [symbol] _{P}, [symbol] _{L}, [symbol] _{P+L}= [symbol] _{P}∪ [symbol] _{L} are the set of atoms in the protein, compound (target compound or training compound), or protein-compound complex respectively. In some embodiments the GCN is configured so that P, L, or P+L atoms can be used as either source atoms (i) or target atoms (j) on a layer-by-layer basis. In some embodiments, the graph convolutional block is based upon a continuous filter convolution

$${f}_{i}^{C O N V} ← {\sum }_{j} {W}^{l} ( {d}_{i j} ) {f}_{j}^{l} .$$

In some embodiments convolutional kernels W^{l}(r) where

$${W}^{l} ( r ) = {\sum }_{n}^{{N}_{b}^{l}} {w}_{n}^{l} {j}_{0} ( r {z}_{0 n} / {R}_{c}^{l} ) ,$$

constructed from a linear combination of zeroth-order spherical Bessel functions j₀(x)=sin x/x, are used. Here, z₀ₙ is the n-th zero of j₀(x), and

$${w}_{n}^{l}$$

are earnable weights, and the number of basis functions is chosen as

$${N}_{b}^{l} = ⌈ {R}_{c}^{l} / {δ}_{l} ⌉ .$$

As an example, in some embodiments, δₗ=0.15625 Å, however, other values may be used. This parametrization is used to ensure that the convolutional kernels vanish at the edge of an internal neighborhood. In some embodiments, forces are not calculated and so a smooth cutoff is not needed here. After the graph convolution step, a Linear—LeakyReLU—Linear layer is included in some embodiments. In some embodiments, a residual connection is not applied at the output of a convolution block. Instead, each convolutional layer takes as input all layers using a bottleneck layer. This was observed to perform better empirically than skip connections from the previous layer of the same set of source atoms. In the embodiment illustrated in FIG. 9C, the network 72 has five graph convolutional blocks. In the first two graph convolutional blocks (902/904), all ligand and receptor atoms are considered and so i,j∈ [symbol] _{P+L}, and R_{c} is set to 5 Å while

$${N}_{f}^{l} = 64$$

filters in some embodiments. For the third graph convolutional block 906, the cutoff radius and filters are increased to R_{c}=7 Å and

$${N}_{f}^{l} = 128$$

filters in some embodiments, however only atoms in the compound (test compound or training compound) are considered as destination atoms in some embodiments (e.g., i∈ [symbol] _{C+L}, but j∈ [symbol] _{L} only). Finally, in the last two layers, 908 and 910 respectively, only ligand atom embeddings are aggregated (i, j∈ [symbol] _{L}) in some embodiments with a cutoff R_{c}=7 Å and

$${N}_{f}^{l} = 128.$$

To construct the initial embedding 74 from this, then, a readout operation is applied to the target protein 38 and compound (test compound or training compound) features independently at each layer:

$${z}_{P}^{l} = {\sum }_{i ∈ {𝒮}_{P}} {f}_{i}^{l} , {z}_{L}^{l} = {\sum }_{i ∈ {𝒮}_{L}} {f}_{i}^{l} .$$

These embeddings are then concatenated together to form the initial embedding for the pose 74:

$${z}_{r e a d} = {⊕}_{l = 0}^{1} {z}_{P}^{l} ⊕ {⊕}_{l = 0}^{5} {z}_{L}^{l} ,$$

where ⊕ represents concatenation. In some embodiments z is constructed from two iterations of Dropout_{0.2}—LeakyReLU—Linear applied to z_{read}.

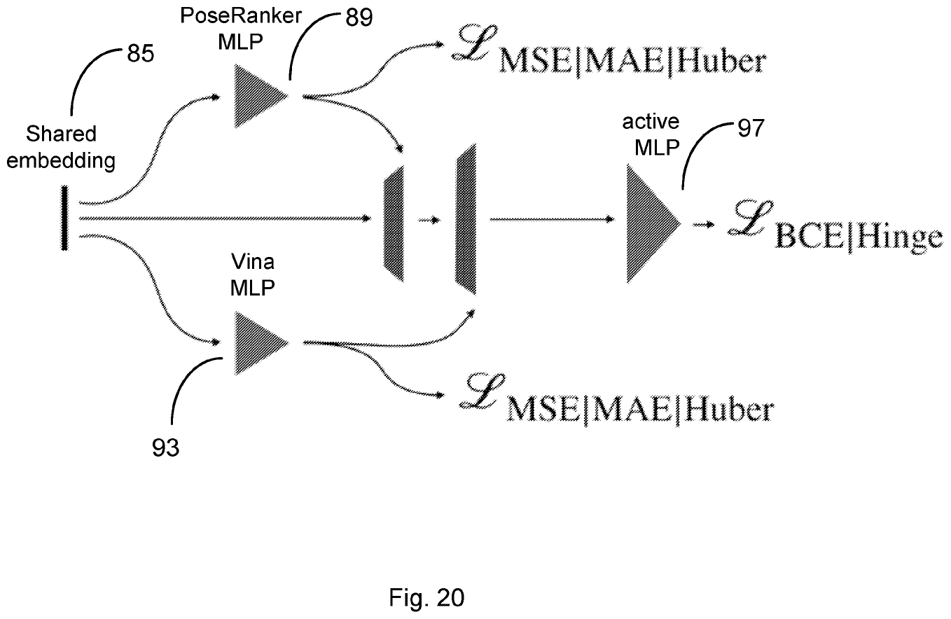

FIG. 20 is a system for characterizing an interaction between a test compound and a target polymer, where the characterization is (i) pose ranking and (ii) Vina score, and activity. In this embodiment, the shared embedding z_{read}=g(f^{0 . . . L}), the PoseRanker score ŷₚₒₛₑ and the Vina score ŷᵥᵢₙₐ were computed by passing z_{read} (initial embedding 74) through separate multi-layer perceptrons yₚₒₛₑ (first model 89 in FIG. 20) and Yvina (optional second model 93 in FIG. 20), respectively. A conditional embedding z′ was then formed by passing z through the condition map:

$$h : {z}_{r e a d} ↦ ( \begin{matrix}{z}_{r e a d} σ ( {\hat{y}}_{p o s e} ) \\ {z}_{r e a d} ( 1 - σ ( {\hat{y}}_{p o s e} ) )\end{matrix} )$$

where σ: x [symbol] (1+e^{-x})⁻¹, and passed to the final MILP p_{active} (third model 97 in FIG. 20) to compute the activity score. Alternatively, z′ may be passed through a second conditional map,

$${h}^{′} : {z}^{′} ↦ ( \begin{matrix}{z}^{′} \\ {\hat{y}}_{v i n a}\end{matrix} )$$

to obtain an embedding z″ that has been conditioned on both the PoseRanker score and the Vina score.

Nonlimiting additional examples of graph convolutional neural networks are disclosed in Behler Parrinello, 2007, “Generalized Neural-Network Representation of High Dimensional Potential-Energy Surfaces,” Physical Review Letters 98, 146401; Chmiela et al., 2017, “Machine learning of accurate energy-conserving molecular force fields,” Science Advances 3(5):e1603015; Schütt et al., 2017, “SchNet: A continuous-filter convolutional neural network for modeling quantum interactions,” Advances in Neural Information Processing Systems 30, pp. 992-1002; Feinberg et al., 2018, “PotentialNet for Molecular Property Prediction,” ACS Cent. Sci. 4, 11, 1520-1530; and Stafford et al., “AtomNet PoseRanker: Enriching Ligand Pose Quality for Dynamic Proteins in Virtual High Throughput Screens,” https://chemrxiv.org/engage/chemrxiv/article-details/614b905e39ef6a1c36268003, each of which is hereby incorporated by reference.

Referring to block 232 of FIG. 2C, in some embodiments the first neural network 72 is an equivariant neural network or a message passing neural network. See Bao and Song, 2020, “Equivariant Neural Networks and Equivarification,” arXiv:1906.07172v4, and Gilmer et al., 2020, “Message Passing Neural Networks,” In: Schütt et al. (eds), Machine Learning Meets Quantum Physics, Lecture Notes in Physics 968, Springer, Cham, each of which is hereby incorporated by reference.

Referring to block 234 of FIG. 2C, in some embodiments the first neural network 72 comprises a plurality of graph convolutional blocks and each block considers connectivity within the respective set of atomic coordinates using a plurality of radial graphs. For instance, in some such embodiments, the first neural network 72 comprises 2, 3, 4, 5, 6, 7, 8, 9, 10, 11, 12, 13, 14, 15, 16, 17, 18, 19, or 20 or more graph convolutional blocks and each block considers connectivity within the respective set of atomic coordinates using a plurality of radial graphs.

Referring to block 236 of FIG. 2C, in some embodiments the first neural network 72 comprises 1×10⁶ parameters. In some embodiments, the first neural network 72 comprises more than 100, 200, 300, 400, 500, 600, 700, 800, 900, 1000, 10,000, 50,000, 100,000 or 1×10⁶ parameters.

Referring to block 238 of FIG. 2C, in some embodiments the corresponding initial embedding 74 comprises a data structure having between 128 and 768 values.

Blocks 240 through 246. Referring to block 240 of FIG. 2C, an attention mechanism 77 is applied to the plurality of initial embeddings (74-1 through 74-P), in concatenated form, thereby obtaining an attention embedding 79. In some embodiments, the plurality of initial embeddings consists of 2, 3, 4, 5, 6, 7, 8, 9, 10, 11, 12, 13, 14, 15, 16, 17, 18, 19, 20, or more than 20 initial embeddings. In some embodiments, an attention mechanism is a mapping of a query (the plurality of initial embeddings in concatenated form) and a set of key-value pairs to an output (the attention embedding 79) where the query, keys, values, and output are all vectors. In some such embodiments, the output (the attention embedding 79) is computed as a weighted sum of the values, where the weight assigned to each value is computed by a compatibility function of the query (the plurality of initial embeddings in concatenated form) with the corresponding key.

Thus, in accordance with block 240, each of the initial embeddings 74 for each of the poses of a compound are concatenated together and applied to an attention mechanism. For instance, if there are five poses 48 for the compound resulting in five initial embeddings 74, the five initial embeddings are concatenated together to form z_{cat} illustrated in FIG. 9A, and this z_{cat} is applied to an attention mechanism 77 to obtain the attention embedding 79. Example attention mechanisms are described in Chaudhari et al., Jul. 12, 2021 “An Attentive Survey of Attention Models,” arXiv:1904-02874v3, and Vaswani et al., “Attention is All You Need,” 31st Conference on Neural Information Processing Systems (NIPS 2017), Long Beach, California, USA, each of which is hereby incorporated by reference. The attention mechanism 77 draws upon the inference that some portions of the pose 48 are more important than others and thus some portions (elements or sets of elements) within the initial embeddings 74 are more important than other portions. For instance, in an example where each initial embedding consists of twenty elements, it may be the case that elements 1-4 and 9-15 contain more information regarding the characterization of an interaction between a compound and a target polymer than elements 5-9 and 16-20. The attention mechanism is trained to discover such observations using training compounds and then apply this learned (trained) observation against the initial embedding 74 of the test compound to form the attention embedding. Thus, the attention mechanism incorporates this notion of relevance by allowing models downstream of the attention mechanism (e.g., the first model 89) to dynamically pay more attention to certain parts of the input embedding (e.g., zₐₜₜₙ, zₚₒₒₗ), that help in performing the task at hand (characterizing an interaction between a test compound and a target polymer) effectively.

Referring to block 244 of FIG. 2D, in some embodiments the plurality of initial embeddings 74 (e.g., in concatenated form) comprises a first plurality of values and the applying the attention mechanism 77 comprises (i) inputting the first plurality of values into an attention neural network thereby obtaining a first plurality of weights, where each weight in the first plurality of weights corresponds to a respective value in the first plurality of values, and (ii) weighting each respective value in the first plurality of values by the corresponding weight in the plurality of weights thereby obtaining the attention embedding. Thus, for instance, consider the case where there are five poses 48 for the compound resulting in five initial embeddings 74, where each of the five initial embeddings contains 20 values. Thus, the concatenation of the five initial embeddings 74 yields a pooled vector (z_{cat} illustrated in FIG. 9A) with 100 elements, each element having a value. As a result of inputting z_{cat} into an attention neural network the attention neural network returns 100 weights, one for each element of z_{cat}, which are then used to adjust the corresponding values in z_{cat} to form zₐₜₜₙ of FIG. 9A. Thus, if z_{cat} has 100 elements, in some embodiments zₐₜₜₙ will also have 100 elements. The value of element 1 of zₐₜₜₙ is the product of (a) the value of element 1 of z_{cat} and (b) the weight for element 1 of z_{cat} returned by the attention neural network, the value of element 2 of zₐₜₜₙ is the product of (a) the value of element 2 of z_{cat} and (b) the weight for element 2 of z_{cat} returned by the attention neural network, and so forth.

Referring to block 246 of FIG. 2D, in some such embodiments the first plurality of weights sum to one (or some other constant value), and each weight in the first plurality of weights is a scalar value between zero and one (or some other constant value). Thus, in the example give above where z_{cat} had 100 elements and the attention neural network therefore returns 100 weights, the 100 weights sum to 1 (or some other constant value) in accordance with block 246 and of these values is a scalar value between 0 and 1 (or some other constant value). In embodiments that have an attention neural network, the attention neural network is jointly trained with at least the first neural network 72 and the first model 89 against the known labels (e.g., pKa, activity, binding score) of a plurality of training compounds. In some embodiments, the plurality of training compounds comprises 25 or more training compounds, 100 or more training compounds, 500 or more training compounds, 1000 or more training compounds, 10,000 or more training compounds, 100,000 or more training compounds or 1×10⁶ training compounds.

Blocks 248 through 256. Referring to block 248 of FIG. 2D, a pooling function 81 is applied to the attention embedding 79 to derive a pooled embedding 85. Examples of pooling functions include, but are not limited to, mean, sum, or max-pooling. Thus, consider the above example where there are five poses 48 for the compound resulting in five initial embeddings 74, where each of the five initial embeddings contains 20 values and thus z_{cat}, and further zₐₜₜₙ, has 100 elements. The pooling function 81 is applied to zₐₜₜₙ to yield zₚₒₒₗ. Consider the case where the pooling function is a mean function. In this example, zₚₒₒₗ will have 20 elements and the first element of zₚₒₒₗ will be the mean of elements 1, 21, 41, 61, and 81 of zₐₜₜₙ, the second element of zₚₒₒₗ will be the mean of elements 2, 22, 42, 62, and 82 of zₐₜₜₙ, . . . , while the twentieth element of zₚₒₒₗ will be the mean of elements 20, 40, 60, 80, and 100 of zₐₜₜₙ. That is, in other words, and referring to block 250 of FIG. 2D, in some such embodiments the pooling function 81 collapses the attention embedding 79 of zₐₜₜₙ into the pooled embedding 85 zₚₒₒₗ by applying a statistical function to combine each corresponding portion of the attention embedding 79 zₐₜₜₙ representing a different pose 48 in the plurality of poses to form the pooled embedding 85.

Now again consider the above example where there are five poses 48 for the compound resulting in five initial embeddings 74, where each of the five initial embeddings contains 20 values and thus z_{cat}, and further zₐₜₜₙ, has 100 elements and the pooling function 81 that is applied to zₐₜₜₙ to yield zₚₒₒₗ is a sum function. In this example, zₚₒₒₗ will have 20 elements and the first element of zₚₒₒₗ will be the sum of elements 1, 21, 41, 61, and 81 of zₐₜₜₙ, the second element of zₚₒₒₗ will be the sum of elements 2, 22, 42, 62, and 82 of zₐₜₜₙ, . . . , while the twentieth element of zₚₒₒₗ will be the sum of elements 20, 40, 60, 80, and 100 of zₐₜₜₙ. That is, in other words, and referring to block 250 of FIG. 2D, in some such embodiments the pooling function 81 collapses the attention embedding 79 zₐₜₜₙ into the pooled embedding 85 by applying a statistical function to combine each corresponding portion of the attention embedding 79 zₐₜₜₙ representing a different pose 48 in the plurality of poses (now weighted by the attention mechanism) to form the pooled embedding 85.

Referring to block 252 of FIG. 2D, in some embodiments the attention embedding 79 zₐₜₜₙ includes a corresponding plurality of values for a corresponding plurality of elements for each respective pose 48 in the plurality of poses and the statistical pooling function is a maximum function that takes a maximum value across corresponding elements of each respective pose 48 represented in the attention embedding 79 zₐₜₜₙ to form the pooled embedding 85. To illustrate, once again consider the above example where there are five poses 48 for the compound resulting in five initial embeddings 74, where each of the five initial embeddings contains 20 values and thus z_{cat}, and further zₐₜₜₙ, has 100 elements and the pooling function 81 that is applied to zₐₜₜₙ to yield zₚₒₒₗ is a max function. In this example, zₚₒₒₗ will have 20 elements and the first element of zₚₒₒₗ will be the maximum value from among elements 1, 21, 41, 61, and 81 of zₐₜₜₙ, the second element of zₚₒₒₗ will be the maximum value from among elements 2, 22, 42, 62, and 82 of zₐₜₜₙ, . . . , while the twentieth element of zₚₒₒₗ will be the maximum value from among elements 20, 40, 60, 80, and 100 of zₐₜₜₙ.

Referring to block 256 of FIG. 2D, in some embodiments the attention embedding 79 includes a corresponding plurality of values for a corresponding plurality of elements for each respective pose 48 in the plurality of poses and the statistical pooling function is an average function that averages the corresponding elements of each respective pose 48 represented in the attention embedding 79 to form the pooled embedding 85. Such an embodiment is similar to the maximum pooling function described above, except that an averaging function instead of a maximum function is applied.

Blocks 258 through 280. Referring to block 258 of FIG. 2E, the pooled embedding 85 is inputted into a first model 89 thereby obtaining a first interaction score of an interaction between the test compound and the target polymer. In some embodiments, the first model 89 comprises more than 400 parameters 91. In some embodiments, the first model 89 comprises more than 400 parameters, more than 1000 parameters, more than 2000 parameters, more than 5000 parameters, more than 10,000 parameters, more than 100,000 parameters, or more than 1×10⁶ parameters. In some embodiments, the amount of information in the pooled embedding that is inputted into the first model 89 coupled with the number of the parameters of the first model 89 results in the performance of more than 10,000 computations, more than 100,000 computations, more than 1×10⁶ computations, more than 5×10⁶ computations, or more than 1×10⁷ computations to calculate the first interaction score.

In some embodiments system 100 or the one more programs hosted, stored, or addressable by system 100, is able to characterize the interaction between a test compound and a target polymer 38. In some such embodiments, this characterization is a discrete (e.g., discrete-binary) activity score. In other words, the characterization is categorical. For instance, referring to block 262 of FIG. 2E, in some embodiments, the first model 89 is a classification task and the first interaction score classifies the interaction between the test compound and the target polymer. In some such embodiments, the characterization (e.g., the first interaction score) is discrete-binary and the computer system provides one value, e.g. a “1”, when the test compound is determined, by in silico methods disclosed herein, to be active against the target polymer and another value, e.g. a “0”, when the test compound is determined to not be active against the target polymer.

In some embodiments, the characterization (e.g., the first interaction score) is on a discrete scale that is other than binary. For instance, in some embodiments, the characterization provides a first value, e.g. a “0”, when the test compound is determined, by the in silico methods disclosed herein, to have an activity that falls below a first threshold, a second value, e.g. a “1”, when the test compound is determined to have an activity that is between a first threshold and a second threshold, and a third value, e.g. a “2”, when the test compound is determined to have an activity that is above the second threshold. In such embodiments, the first and second threshold are predetermined and constant for a particular experiment (e.g., for a particular evaluation of a particular database of test compounds against a particular target polymer) and are chosen to have values that prove to be useful in identifying suitable test compounds from a database of test compounds for activity against the test polymer. For instance, in some embodiments, any of the thresholds disclosed herein are designed to identify 0.1 percent or fewer, 0.5 percent or fewer, 1 percent or fewer, 2 percent or fewer, 5 percent or fewer, 10 percent or fewer, 20 percent or fewer, or 50 percent or fewer of a database of test compounds as being active against the target polymer, where the database of test compounds comprises 100 or more compounds, 1000 or more compounds, 10,000 or more compounds, 100,000 or more compounds, 1×10⁶ compounds, 10×10⁶ compounds or more.

In alternative embodiments, system 100 or the one more programs hosted, stored, or addressable by system 100, is able to characterize the interaction between a test compound and a target polymer 38 as an activity on a continuous scale. That is, system 100 or the one more programs hosted, stored, or addressable by system 100, provides a number on a continuous scale that indicates the activity of the test compound against the target polymer. The activity value on the continuous scale is useful, for instance, in comparing the activity of each test compound in a database of test compounds against the target polymer that was assigned by the trained spatial data evaluation module 36. As an example, referring to block 260 of FIG. 2E, in some embodiments, the first model 89 is a regression task and the first interaction score quantifies the interaction between the test compound and the target polymer.

The disclosed systems and methods are not limited to characterizing the interaction between a test compound and a target polymer 38 as an activity on a continuous scale or discrete scale. In alternative embodiments, system 100 or the one more programs hosted, stored, or addressable by system 100 characterize the interaction between a test compound and a target polymer as an IC₅₀, EC₅₀, Kd, KI, or pKI of the test compound against the target polymer on a continuous scale or a discrete (categorical) scale.

While a binary-discrete scale and a discrete-scale with three possible outcomes has been identified, the present disclosure is not limited to these two examples of discrete-scales for the characterization of the interaction between a test compound and a target polymer 38. In fact, any discrete scale can be used for the characterization of the interaction between a test compound and a target polymer 38 including, as non-limiting examples, a discrete scale with 4, 5, 6, 7, 8, 9, 10, 11, 12, 13, 14, 15, 16, 17, 18, 19, or 20 different outcomes.

Referring to block 264 of FIG. 2E, in some embodiments, the first interaction score represents a binding coefficient of the test compound to the target polymer.

Referring to block 266 of FIG. 2E, in some embodiments, the first interaction score is an IC₅₀, EC₅₀, Kd, KI, or pKI for the test compound with respect to the target polymer. IC₅₀, EC₅₀, Kd, KI, and pKI are generally described in Huser ed., 2006, High-Throughput-Screening in Drug Discovery, Methods and Principles in Medicinal Chemistry 35; and Chen ed., 2019, A Practical Guide to Assay Development and High-Throughput Screening in Drug Discovery, each of which is hereby incorporated by reference.

Referring to block 270 of FIG. 2F, in some embodiments, the first interaction score is a binary score, where a first value for the binary score represents an IC₅₀, EC₅₀, Kd, KI, or pKI for the test compound with respect to the target polymer that is above a first threshold, and a second value for the binary score represents an IC₅₀, EC₅₀, Kd, KI, or pKI for the test compound with respect to the target polymer that is below the first threshold.

Referring to block 272 of FIG. 2F, in some embodiments, the first interaction score represents an in silico pose quality score of the test compound to the target polymer.

Referring to block 274 of FIG. 2F, in some embodiments, the first model 89 is a fully connected second neural network, also known as a multilayer perceptron (MLP). In some embodiments, a MLP is a class of feedforward artificial neural network (ANN) comprising at least three layers of nodes: an input layer, a hidden layer and an output layer. In such embodiments, except for the input nodes, each node is a neuron that uses a nonlinear activation function. More disclosure on suitable MLPs that serve as the first model 89 in some embodiments of the present disclosure is found in Vang-mata ed., 2020, Multilayer Perceptrons: Theory and Applications, Nova Science Publishers, Hauppauge, New York, which is hereby incorporated by reference.

Referring to block 276 of FIG. 2F, in some embodiments, the pooled embedding 85 is also inputted into a second model 93 thereby obtaining a second interaction score of an interaction between the test compound and the target polymer. In such embodiments, the first model 89 is a first fully connected neural network, the second model 93 is a second fully connected neural network, the first interaction score represents an in silico pose quality score of the test compound to the target polymer, and the second interaction score represents an in silico pose quality score of the test compound to the target polymer.

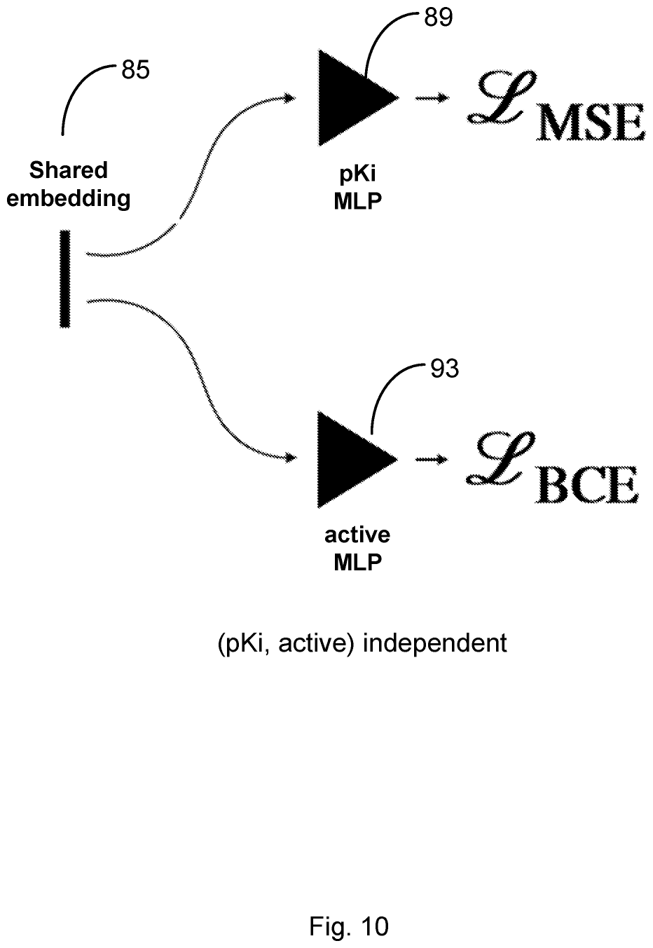

FIG. 10 is a system for characterizing an interaction between a test compound and a target polymer, where the characterization is (i) binary-discrete activity and (ii) pKi, and where the system is trained using training compounds for which activity and pKi is known, in accordance with one embodiment of the present disclosure. In this training, a multi-task loss function is calculated and minimized over all tasks (in the case of FIG. 10 binary-discrete activity and pKi) and a minibatch of Nb training compounds:

$$ℒ = {\sum }_{i = 1}^{{N}_{b}} {\sum }_{t = 1}^{\overline{T}} {w}_{t} {l}_{t} ( {y}_{it} , {\hat{y}}_{i t} )$$

where wₜ and lₜ denote the weight and loss for task t, respectively, ŷᵢₜ is the output of each task t for a given training compound i, and yᵢₜ is the pooled embedding 81 that is passed to each task t. The pooled embedding is yᵢₜ is denoted as such because there is no requirement that each task (e.g., first model 89, second model 93, third model 97, etc.) receive the same pooled embedding 85. In some embodiments, although not shown in FIG. 10, each task (e.g., first model 89, second model 93, third model 97, etc.) receives a different pooled embedding 85 from a different pooling function 81. Thus, architectures are contemplated in which the attention embedding 79 is passed to more than one pooling function to arrive at more than one pooled embedding 85, and that each of the more than one pooled embedding is passed to a different task t (e.g., first model 89, second model 93, third model 97, etc.). In other embodiments, there is a single pooling function 81 that produces a single pooled embedding that is then passed to one or more tasks (e.g., first model 89, second model 93, third model 97, etc.).

In the system of FIG. 10, the pKi model and the activity model are independent of each other. In some embodiments, the pKi model is trained as a regression task using a loss function such as mean squared error against the pKi values of the training compounds, whereas the activity model is trained as a classification task using a loss function such as binary cost entropy against the known binary-discrete activity values of the training compounds.

As illustrated in FIG. 10, is some embodiments the pooled embedding 85 is inputted into both a first model 89 (to provide a characterization of the interaction between the test compound and the target polymer in the form of a calculated pKi value) as well as a second model 93 (to provide a characterization of the interaction between the test compound and the target polymer in the form of an activity of the test compound with respect to the target polymer 38). Thus, in the embodiment illustrated in FIG. 10, the characterization of the interaction between the test compound and the target polymer is both an pkI score (e.g., a discrete-binary score or a scalar score) and an activity score (e.g. a classification as “good binder”, “bad binder,” etc.). While the second model 89 computes pKi in the embodiment illustrated in FIG. 10, it will be appreciated that in other embodiments having the topology of FIG. 10, the second model computes IC₅₀, EC₅₀, Kd, or KI instead of pKI.

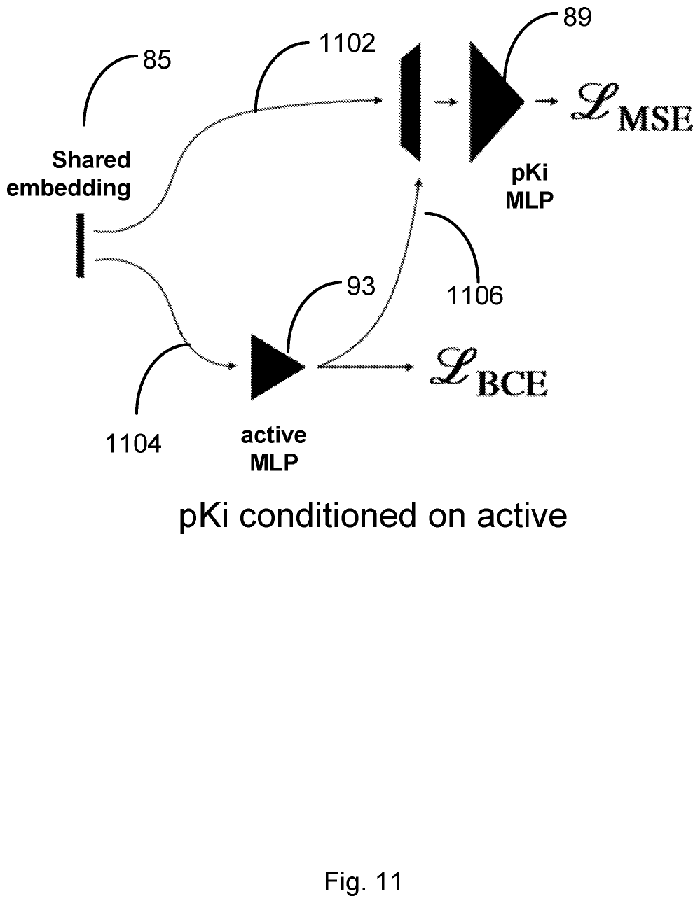

FIG. 11 is a system for characterizing an interaction between a test compound and a target polymer, where the characterization is pKi, and where the pKi is conditioned, in part, on activity, and where the system is trained using the known pKi and activity of training compounds, in accordance with one embodiment of the present disclosure. In the system of FIG. 11, the pKi model is conditioned on the activity model. In some embodiments, the pKi model is trained as a regression task using a loss function such as mean squared error against the pKi values of the training compounds, whereas the activity model is trained as a classification task using a loss function such as binary cost entropy against the activity values of the training compounds.

As illustrated in FIG. 11, is some embodiments the pooled embedding 85 is inputted into both the first model 89 (through edge 1102) as well as the second model 93 (through edge 1104). Further, the output of the second model 93, which is a calculation of the activity of the test compound with respect to the target polymer, is inputted into the first model 89 through edge 1106. In some embodiments, this characterization provided by the second model 93 is an activity score of the test compound. In some embodiments, this activity score is a discrete-binary score, for instance where a “1” indicates the test compound is active against the target polymer and a “0” indicates that the test compound is inactive against the target polymer. In some embodiments, the activity score provided by the second model 93 is scalar. Thus, the first model 89 receives both the output of the second model 93 and the pooled embedding 85. The first model 89 uses both of these inputs to determine the characterization of the interaction between the test compound and the target polymer (e.g., in the form of a pKi of the test compound with respect to the target polymer 37). The conditioning of the pKi calculation of the first model 89 on both the pooled embedding 85 and the second model 93 serves to improve the performance of the first model 89 at characterizing test compounds. While the second model 89 computes pKi in the embodiment illustrated in FIG. 11, it will be appreciated that in other embodiments having the topology of FIG. 11, the second model computes IC₅₀, EC₅₀, Kd, or KI instead of pKI.

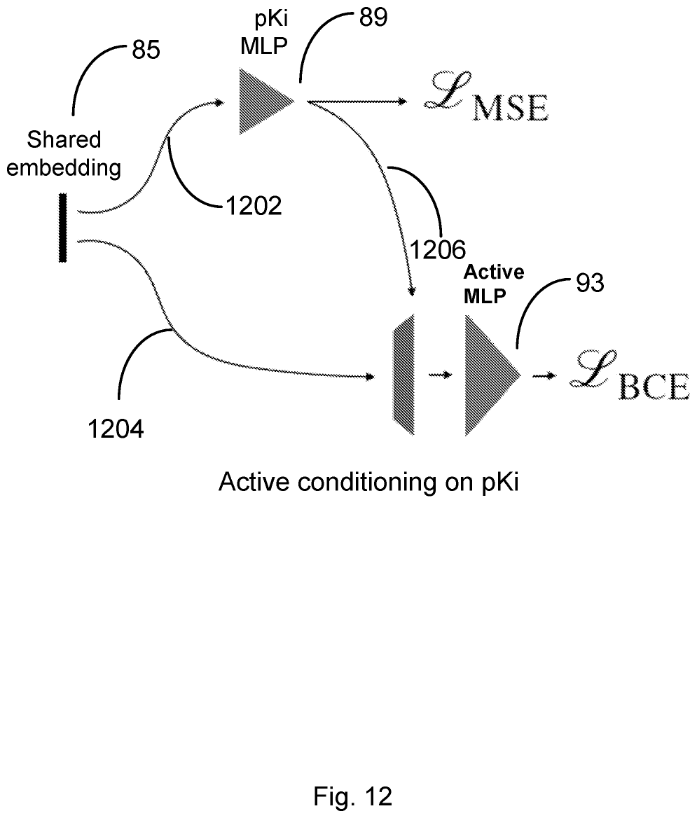

As illustrated in FIG. 12, is some embodiments the pooled (shared) embedding 85 is inputted into both the first model 89 (through edge 1202) as well as the second model 93 (through edge 1204). Further, the output of the first model 89, which is a calculation of the pKi of the test compound with respect to the target polymer, is inputted into the second model 93 through edge 1206. Thus, the second model 93 receives both the output of the first model 89 and the pooled embedding 85. The second model 93 uses both of these inputs to determine the characterization of the interaction between the test compound and the target polymer (e.g., in the form of an activity score of the test compound). In some embodiments, this activity score is a discrete-binary score, for instance where a “1” indicates the test compound is active against the target polymer and a “0” indicates that the test compound is inactive against the target polymer. In some embodiments, the activity score provided by the second model 93 is scalar. The conditioning of the activity score of the second model 93 on both the pooled embedding 85 and the output of the first model 93 serves to improve the performance of the second model 83 at characterizing test compounds. While the first model 89 computes pKi in the embodiment illustrated in FIG. 12, it will be appreciated that in other embodiments having the topology of FIG. 12, the first model 89 computes IC₅₀, EC₅₀, Kd, or KI instead of pKI.

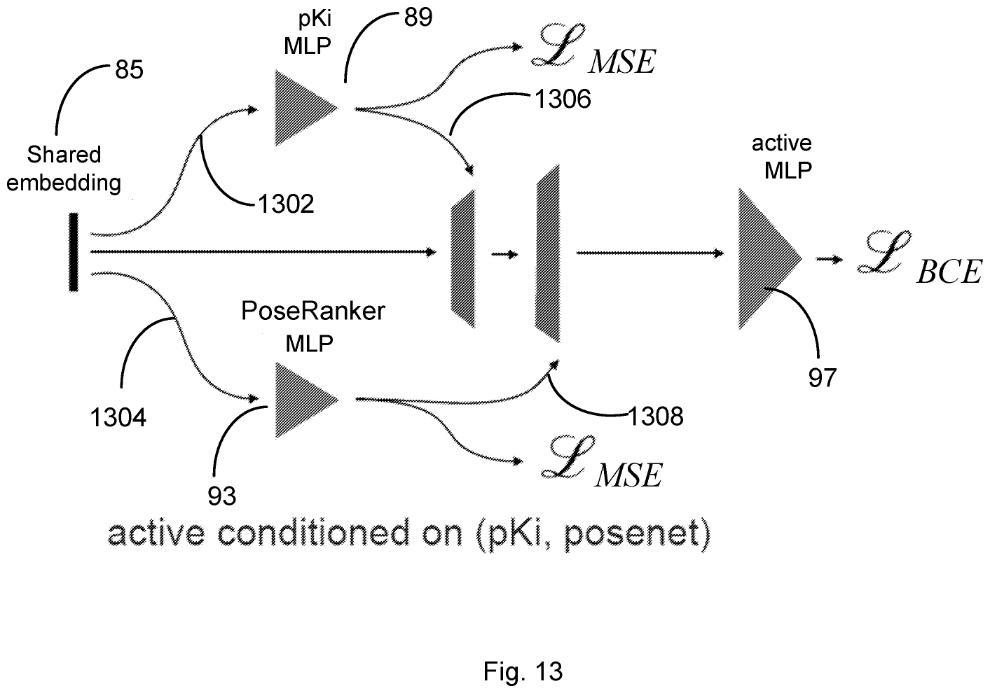

Referring to block 278 of FIG. 2F, in some such embodiments the first interaction score and the second interaction score is inputted into a third model 97 to obtain a third interaction score. In some such embodiments, the third model 97 is a third fully connected neural network. Referring to block 280 of FIG. 2F, in some such embodiments the third interaction score is discrete-binary activity score with a first value when the test compound is determined by the third model 97 to be inactive and a second value when the test compound is determined by the third model 97 to be active. For instance, FIG. 13 illustrates a system for characterizing an interaction between a test compound and a target polymer, where the characterization is activity (through an activity model 97), and where the activity is conditioned, in part, on both pKi (through a pKi model 89) and binding mode score (through a PoseRanker model 93), and where the pKi model is trained using the known pKi values for the training compounds and the PoseRanker model is trained using binding mode scores for training compounds, in accordance with one embodiment of the present disclosure. For purposes of training the activity model 97 of FIG. 13, in some embodiments each training compound is labeled as active if its pKi (or IC50) is less than 10 μM; otherwise it is labeled as inactive. For purposes of training the PoseRanker model 93, in some embodiments the binding mode scores of the training compounds are obtained by docking the training compounds with a docking program such as CUina μMorrison et al., 2020, “CUina: An Efficient GPU Implementation of AutoDock Vina. August 2020. URL https://blog.atomwise.com/efficient-gpu-implementation-of-autodock-vina.] and then ranking each pose of each docked compound with PoseRanker model [Stafford et al., 2021, “AtomNet PoseRanker: Enriching Ligand Pose Quality for Dynamic Proteins in Virtual High Throughput Screens,” doi: 10.33774/chemrxiv-2021-t6xkj. URL https://chemrxiv.org/engage/chemrxiv/article-details/614b905e39ef6a1c36268003. Thus, in such embodiments the binding mode score used for training the PoseRanker model 93 is the PoseRanker ranking.

In the system of FIG. 13, the activity model 97 is conditioned on both a pKi model 89 and a PoseRanker model 93. In some embodiments, the pKi model and the PoseRanker model (Stafford et al., 2022, “AtomNet PoseRanker: Enriching Ligand Pose Quality for Dynamic Proteins in Virtual High-Throughput Screens,” Journal of Chemical Information and Modeling 62, pp. 1178-1189, which is hereby incorporated by reference) is trained as a regression task using a loss function such as mean squared error, whereas the activity model is trained as a classification task using a loss function such as binary cost entropy.

Thus, as illustrated in FIG. 13, is some embodiments the pooled (shared) embedding 85 is inputted into both the first model 89 (through edge 1302) as well as the second model 93 (through edge 1304). Further, the output of the first model 89, which is a calculation of the pKi of the test compound with respect to the target polymer, is inputted into a third model 97 through edge 1306. Further, the output of the second model 93, which is a calculation of the quality of the poses of the test compound with respect to the target polymer as represented by the pooled embedding 85, is also inputted into the third model 97 through edge 1308. In FIG. 13, the second model is termed a PoseRanker model. Throughout the present disclosure, the terms “PoseNet” and “PoseRanker” are used interchangeably. The PoleRanker (PoseNet) model is described in further detail in Stafford et al., 2022, “AtomNet PoseRanker: Enriching Ligand Pose Quality for Dynamic Proteins in Virtual High-Throughput Screens,” Journal of Chemical Information and Modeling, Volume 62, pp. 1178-1189, which is hereby incorporated by reference. In addition the pooled embedding 85 is inputted into the third model. Thus, the third model 97 receives the output of the first model 89, the output of the second model 93, and the pooled embedding 85. The third model 97 uses all of these inputs to determine the characterization of the interaction between the test compound and the target polymer (e.g., in the form of an activity score of the test compound). In some embodiments, this activity score is a discrete-binary score, for instance where a “1” indicates the test compound is active against the target polymer and a “0” indicates that the test compound is inactive against the target polymer. While the first model 89 computes pKi in the embodiment illustrated in FIG. 13, it will be appreciated that in other embodiments having the topology of FIG. 13, the first model 89 computes IC₅₀, EC₅₀, Kd, or KI instead of pKI.

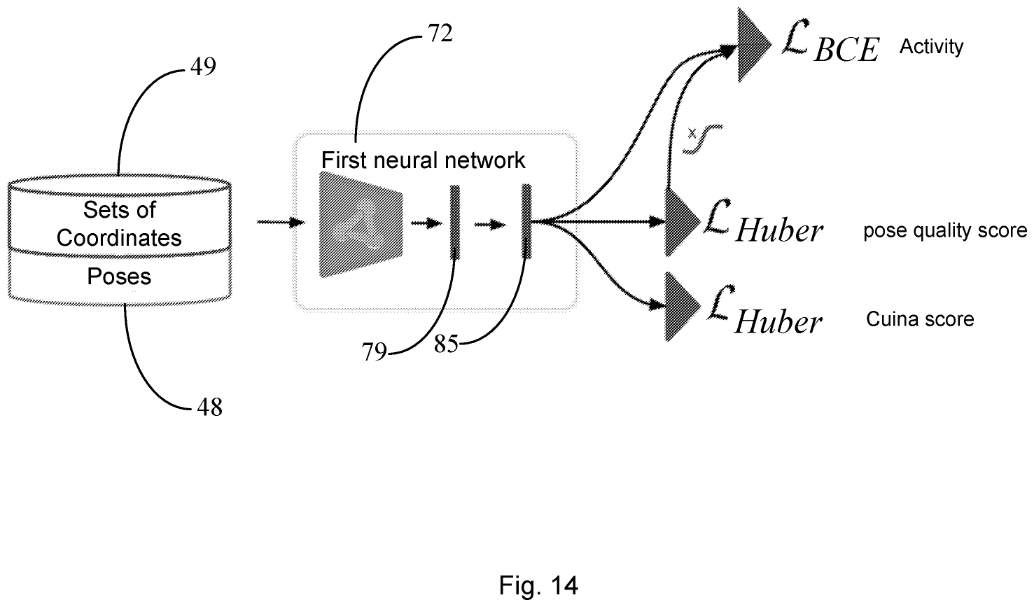

FIG. 14 is a system for characterizing an interaction between a test compound and a target polymer, where the characterization is activity and two different compound binding mode scores, and where the system is trained using training compounds with known activity scores, in accordance with one embodiment of the present disclosure. In the system of FIG. 14, the activity model is conditioned on a pose quality score model. In some embodiments, the pose quality model is trained as a regression task using a loss function such as mean squared error, whereas the activity model is trained as a classification task using a loss function such as binary cost entropy.

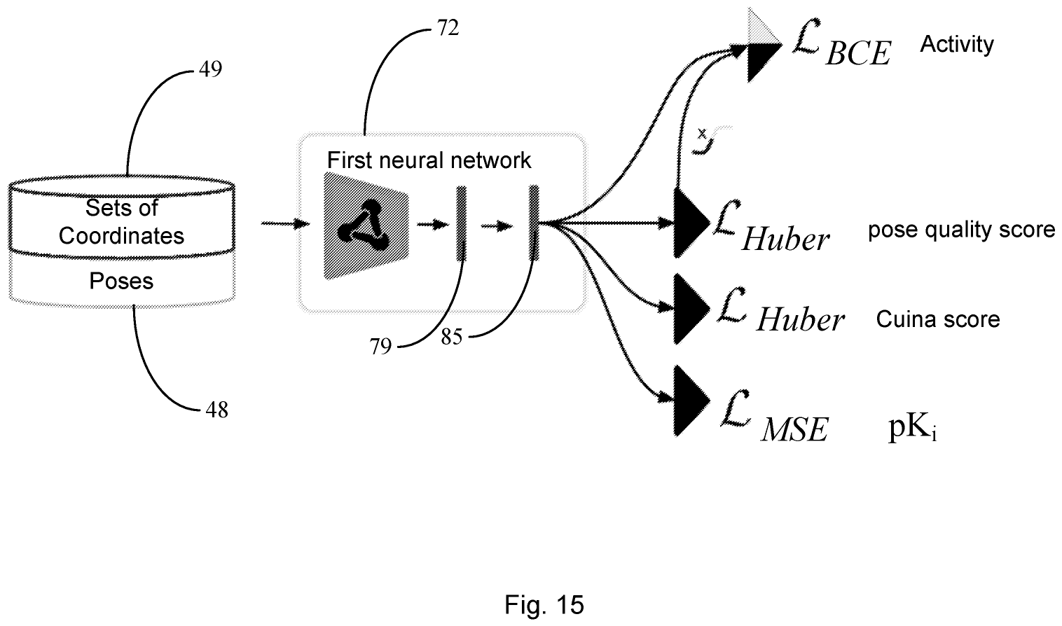

FIG. 15 is a system for characterizing an interaction between a test compound and a target polymer, where the characterization is activity, two different compound binding mode scores and pKi, and where the system is trained using coupled positive and negative poses for training compounds, in accordance with one embodiment of the present disclosure. In the system of FIG. 15, the activity model is conditioned on a pose quality score model. In some embodiments, the pose quality model is trained as a regression task using a loss function such as mean squared error, whereas the activity model is trained as a classification task using a loss function such as binary cost entropy.

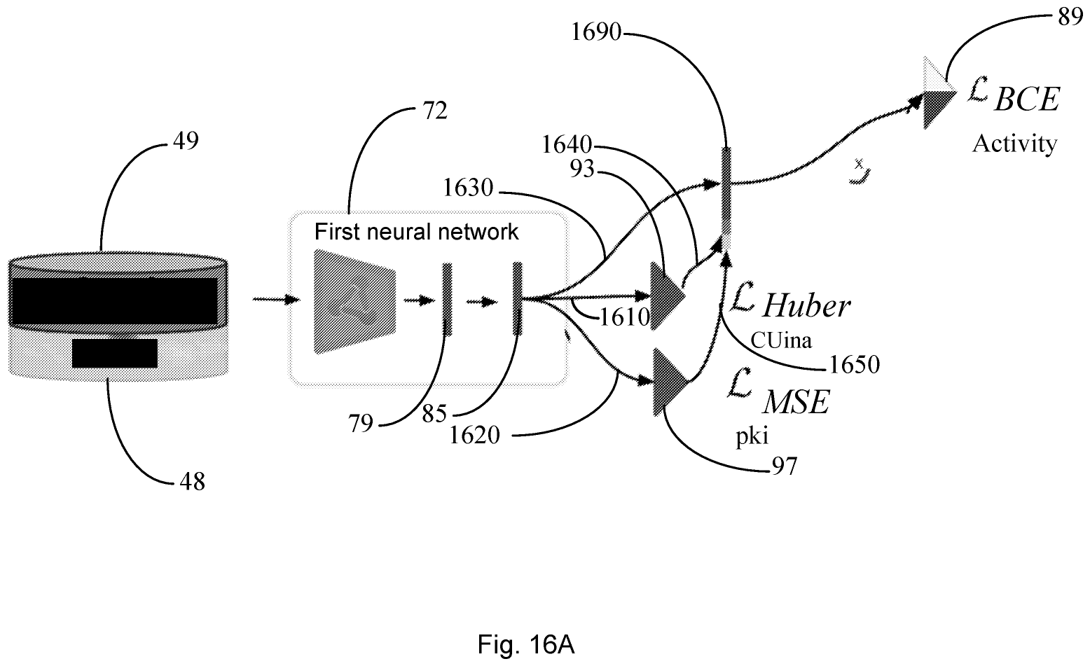

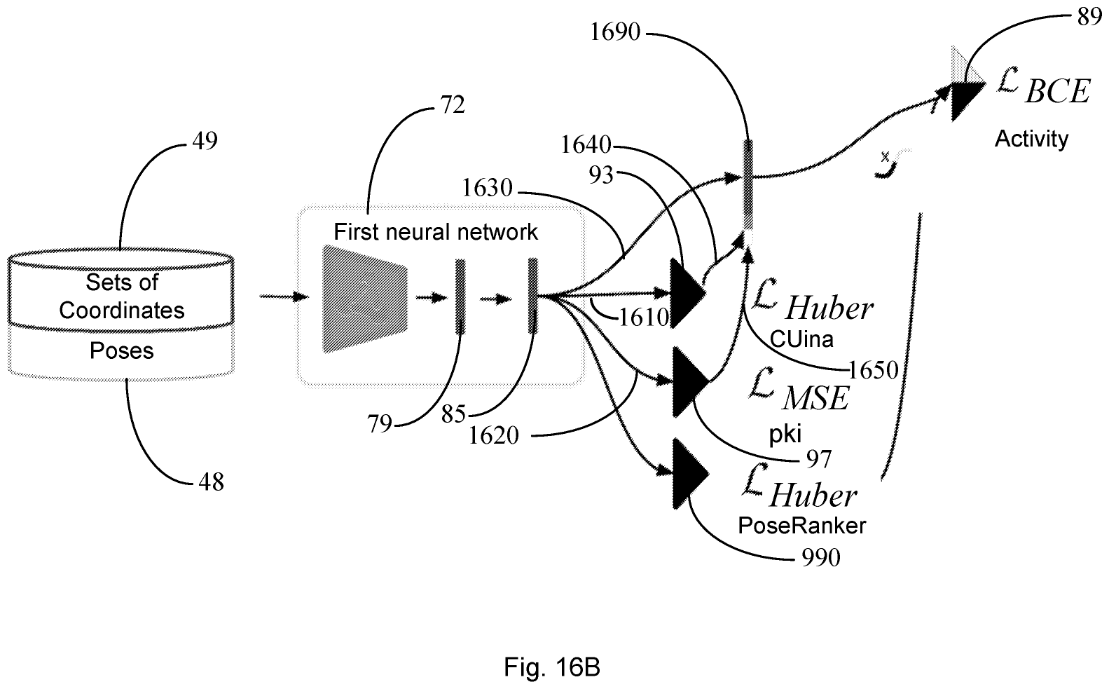

In the embodiment illustrated in FIG. 16A, the poses, for instance in the form of the sets of atomic coordinates 49 or the vectors 54, of the test compound are introduced into the first neural network 72 to ultimately yield the pooled embedding 85 in accordance with FIG. 9A. This pooled embedding 85 is inputted into the first model 89 (through edge 1630), the second model 93 (through edge 1610), and the third model 97 (through edge 1620). Further, the output of the second model 93 (which is a calculation of the interaction score, such as pose quality score, etc.) of the test compound is inputted into the first model 89 through edge 1640. Further still, the output of the third model 97 (which is a calculation of the interaction score, such as pKi, etc.) of the test compound is inputted into the first model 89 through edge 1650. Thus, the first model receives the output of the third model, the second model, and the pooled embedding 84. The first model 89 uses each of these inputs collectively to determine the characterization of the interaction between the test compound and the target polymer. In some embodiments this characterization is an activity score of the test compound. In some embodiments, this activity score is a discrete-binary score, for instance where a “1” indicates the test compound is active against the target polymer and a “0” indicates that the test compound is inactive against the target polymer. In some embodiments, the activity score provided by the first model 89 is scalar.

In some embodiments, with reference to FIG. 16A, the pooled embedding 85 is used to predict three outputs: the activity (through the first model 89), a CUina pose quality score (through the second model 93), and a pKi score (through the third model 97). For description of fully connected CUina pose quality models, see Morrison et al., “Efficient GPU Implementation of AutoDock Vina,” COMP poster 3432389, which is hereby incorporated by reference. This is performed in two stages in the embodiment illustrated in FIG. 16A. First, the CUina and pKi score predictions are computed by passing the pooled embedding 85 through the second model 93 and the third models 97. Second, a conditioned embedding 1690 is formed by concatenating (i) the pooled embedding 85, (ii) the resulting second model 93 score prediction from the first stage, and (iii) the third model 97 score prediction from the first stage. This embedding 1690 is then passed to the first model 89, which is in the form of a multilayer perceptron, to compute the activity prediction for the test compound. In some embodiments, rather than simply concatenating (i) the pooled embedding 85, (ii) the resulting second model 93 score prediction from the first stage, and (iii) the third model 97 score prediction from the first stage, the embedding 1690 represents a multiplication of the three components against each other, or some other mathematical combination of these three components. In such embodiments, the product of the multiplication of the three components, or some other mathematical combination of the three components, is inputted into the third model as embedding 1690. In some embodiments, the embedding 1690, rather than concatenating the three components, transforms each of the three sources and this transformation serves as input to the first model 89. More generally, embedding 1690 is capable of performing any mathematical function on all or any part of any of the inputs to embedding 1690, including but not limited to multiplication, concatenation, linear or nonlinear transformation in order to form a condition embedding that is passed on to the first model 72. While the third model 97 estimates pKi in the embodiment illustrated in FIG. 16A, it will be appreciated that in other embodiments having the topology of FIG. 16A, the third model 97 estimates IC₅₀, EC₅₀, Kd, or KI of the test compound with respect to the target polymer instead of pKI.

Referring to FIG. 16B, it is possible to condition the first model 89 on additional models as well. Thus, in FIG. 16B, the first model 89 is conditioned, in addition to the pooled embedding 85, on the output of a second model 93 that provides a CUina score of the test compound with respect to the target polymer, a third model 97 that provides a pKi score of the test compound with respect to the target polymer, and a fourth model 990 that provides a PoseRanker score of the test compound with respect to the target polymer. While the third model 97 estimates pKi in the embodiment illustrated in FIG. 16B, it will be appreciated that in other embodiments having the topology of FIG. 16B, the third model 97 estimates IC₅₀, EC₅₀, Kd, or KI of the test compound with respect to the target polymer instead of pKI. Referring to FIG. 16B, in some such embodiments the first model, the second model, the third model, and the fourth model 990 are each a fully connected neural network. Such fully connected neural networks are also known as multilayer perceptrons (MLP). In some embodiments, a MLP is a class of feedforward artificial neural network (ANN) comprising at least three layers of nodes: an input layer, a hidden layer and an output layer. In such embodiments, except for the input nodes, each node is a neuron that uses a nonlinear activation function. More disclosure on suitable MLPs that serve as the first model 72 in some embodiments is found in Vang-mata ed., 2020, Multilayer Perceptrons: Theory and Applications, Nova Science Publishers, Hauppauge, New York, which is hereby incorporated by reference. Referring to FIG. 16B, in some embodiments the corresponding activity score provided by the first model 89 is a binary activity score. For instance, in some embodiments, an activity score having a value of “1” means that the test compound is “active” at inhibiting an activity or function (e.g., enzymatic activity) of the target polymer and an activity score of “0” means that the test compound does not inhibit an activity or function of the target polymer.

Representative test compounds and training compounds. Several different architectures or systems have been described for characterizing an interaction between a test compound and a target polymer. Before each such architecture or system can be used to characterize an interaction between a test compound and the target polymer, it is trained against training compounds. The significant difference between a test compound and training compounds is that the training compounds are labeled (e.g., with complementary binding data against the target polymer obtained from wet lab binding assays, etc.) and such labeling is used to train the first neural network 72, attention mechanism 77, first model 89, second model 93, and optional third and subsequent models, whereas each test compound is either not labeled or the labels are not used and the first neural network 72, attention mechanism 77, first model 89, second model 93, and optional third and subsequent models of the present disclosure are used to characterize an interaction between each test compound and the target polymer. In other words, the training compounds are already characterized by labels (characterization of the interaction between the training compounds and the target polymer), and such characterization is used to train the models of the present disclosure so that they may characterize an interaction between the test compounds and the target polymer. The interaction between the test compounds and the target polymer are typically not characterized prior to application of the first neural network 72 and other models of the present disclosure. In typical embodiments, the characterizations of the interactions between the training compounds and the target polymer that are available is binding data against the target polymer 38 obtained by wet lab binding assays.

Training the predictive model. In some embodiments a predictive model in accordance with the present disclosure, such as the predictive model collectively depicted in FIG. 9A, is trained to receive the geometric data input for a compound (e.g., poses 48 for the compounds) and to output a characterization of the interaction between the compound and the target polymer. For instance, in some embodiments, each of the several poses for each of a plurality of training compounds (e.g., 50 or more training compounds, 100 or more training compounds, 1000 or more training compounds, 100,000 or more training compounds), which have known binding data against the target polymer are sequentially run through the model illustrated in FIG. 9A and the model provides a single value for each respective training compound.

In some such embodiments, the systems of the present disclosure (e.g., the system illustrated in FIG. 9A) outputs one of two possible activity classes for each training compounds against a given target polymer. For instance, the single value provided for each respective training compound by the systems of the present disclosure is in a first activity class (e.g., binders) when it is below a predetermined threshold value and is in a second activity class (e.g., nonbinders) when the number is above the predetermined threshold value. The activity classes assigned by the systems of the present disclosure are compared to the actual activity classes as represented by the training compound binding data. In typical non-limiting embodiments, such training compound binding data is from independent web lab binding assays. Errors in activity class assignments made by the systems of the present disclosure, as verified against the binding data, are then back-propagated through the parameters of the each of the models of the systems of the present disclosure (e.g., first neural network 72, attention mechanism 77, and/or first model 89, etc.) in order to train the system. In an exemplary embodiment, a model of the present disclosure is trained against the errors in the activity class assignments made by the model, in view of the binding data, by stochastic gradient descent with the AdaDelta adaptive learning method (Zeiler, 2012 “ADADELTA: an adaptive learning rate method,” CoRR, vol. abs/1212.5701, which is hereby incorporated by reference), and the back propagation algorithm provided in Rumelhart et al., 1988, “Neurocomputing: Foundations of research,” ch. Learning Representations by Back-propagating Errors, pp. 696-699, Cambridge, MA, USA: MIT Press, which is hereby incorporated by reference. In some such embodiments, the two possible activity classes are respectively a binding constant greater than a given threshold amount (e.g., an IC₅₀, EC₅₀, or KI for the training compound with respect to the target polymer that is greater than one nanomolar, ten nanomolar, one hundred nanomolar, one micromolar, ten micromolar, one hundred micromolar, or one millimolar) and a binding constant that is below the given threshold amount (e.g., an IC₅₀, EC₅₀, or KI for the training compound with respect to the target compound that is less than one nanomolar, ten nanomolar, one hundred nanomolar, one micromolar, ten micromolar, one hundred micromolar, or one millimolar).

In some embodiments, the systems of the present disclosure output one of a plurality of possible activity classes (e.g., three or more activity classes, four or more activity classes, five or more activity classes) for each training compound against a given target polymer. For instance, the single value provided for each respective training compound by the systems and methods of the present disclosure is in a first activity class when the number falls into a first range, is in a second activity class when the number falls into a second range, is in a third activity class when the number falls into a third range, and so forth. The activity classes assigned by the systems of the present disclosure are compared to the actual activity classes as represented by the training compound binding data of other forms of training data. Errors in activity class assignments made by the systems of the present disclosure, as verified against the binding data (or other forms of measured or independently calculated data), are then used to train the systems of the present disclosure using the techniques discussed above. In some embodiments, each respective classification in the plurality of classifications is an IC₅₀, EC₅₀, pkA, or KI range for the training compound with respect to the target polymer.

In some embodiments, classification of a plurality of training compounds by the systems of the present disclosure is compared to the training data (e.g., binding data or other independently measured data for the training compounds) using non-parametric techniques. For instance, the systems of the present disclosure are used to rank order the plurality of training compounds with respect to a given property (e.g., binding against a given target polymer) and this rank order is compared to the rank order provided by the training data that is acquired by wet lab binding assays for the plurality of training compounds. This gives rise to the ability to train the systems of the present disclosure on the errors in the calculated rank order using the system error correction techniques discussed above. In some embodiments, the error (differences) between the ranking by the training compounds by the systems of the present disclosure and the ranking of the training compounds as determined by the binding data (or other independently measured data for the training compounds) is computed using a Wilcoxon Mann Whitney function (Wilcoxon signed-rank test) or other non-parametric test and this error is used to further train the systems of the present disclosure (e.g., first neural network 72, attention mechanism 77 and/or first model 89, etc.).

In some embodiments, model training may involve modifying the parameters of one/or more component models. The parameters may be further constrained with various forms of regularization such as L1, L2, weight decay, and dropout.

In an embodiment, any of the models disclosed herein may optionally, where training data is labeled (e.g., with binding data), have their parameters (e.g., weights) tuned (adjusted to potentially minimize the error between the system's predicted binding affinities and/or categorizations and the training data's reported binding affinities and/or categorizations). Various methods may be used to minimize the error function, such as gradient descent methods, which may include, but are not limited to, log-loss, sum of squares error, hinge-loss methods. These methods may include second-order methods or approximations such as momentum, Hessian-free estimation, Nesterov's accelerated gradient, adagrad, etc. Unlabeled generative pretraining and labeled discriminative training may also be combined.

Using a convolutional neural network as the first neural network 72. In order to characterize an interaction between a test compound and a target polymer, in some embodiments a voxel map 52 is created for a respective pose 48 of a compound. In some embodiments, the voxel map 52 is created by (i) sampling the compound in a pose 48, and the target polymer 38 on a three-dimensional grid basis thereby forming a corresponding three-dimensional uniform space-filling honeycomb comprising a corresponding plurality of space filling (three-dimensional) polyhedral cells and (ii) populating, for each respective three-dimensional polyhedral cell in the corresponding plurality of three-dimensional cells, a voxel (discrete set of regularly-spaced polyhedral cells) in the respective voxel map based upon a property (e.g., chemical property) of the respective three-dimensional polyhedral cell. Thus, for a particular pose 48 of a particular compound, a corresponding voxel map 52 is created. Examples of space filling honeycombs include cubic honeycombs with parallelepiped cells, hexagonal prismatic honeycombs with hexagonal prism cells, rhombic dodecahedra with rhombic dodecahedron cells, elongated dodecahedra with elongated dodecahedron cells, and truncated octahedra with truncated octahedron cells.

In some embodiments, the space filling honeycomb is a cubic honeycomb with cubic cells and the dimensions of such voxels determine their resolution. For example, a resolution of 1 Å may be chosen meaning that each voxel, in such embodiments, represents a corresponding cube of the geometric data with 1 Å dimensions (e.g., 1 Å×1 Å×1 Å in the respective height, width, and depth of the respective cells). However, in some embodiments, finer grid spacing (e.g., 0.1 Å or even 0.01 Å) or coarser grid spacing (e.g. 4 Å) is used, where the spacing yields an integer number of voxels to cover the input geometric data. In some embodiments, the sampling occurs at a resolution that is between 0.1 Å and 10 Å. As an illustration, for a 40 Å input cube, with a 1 Å resolution, such an arrangement would yield 40*40*40=64,000 input voxels.

In some embodiments, a characteristic of an atom incurred in the sampling (i) is placed in a single voxel in the respective voxel map, and each voxel in the plurality of voxels represents a characteristic of a maximum of one atom. In some embodiments, the characteristic of the atom consists of an enumeration of the atom type. As one example, some embodiments of the disclosed systems and methods are configured to represent the presence of every atom in a given voxel of the voxel map 52 as a different number for that entry, e.g., if a carbon is in a voxel, a value of 6 is assigned to that voxel because the atomic number of carbon is 6. However, such an encoding could imply that atoms with close atomic numbers will behave similarly, which may not be particularly useful depending on the application. Further, element behavior may be more similar within groups (columns on the periodic table), and therefore such an encoding poses additional work for the convolutional neural network 24 to decode.

In some embodiments, the characteristic of the atom is encoded in the voxel as a binary categorical variable. In such embodiments, atom types are encoded in what is termed a “one-hot” encoding: every atom type has a separate channel. Thus, in such embodiments, each voxel has a plurality of channels and at least a subset of the plurality of channels represent atom types. For example, one channel within each voxel may represent carbon whereas another channel within each voxel may represent oxygen. When a given atom type is found in the three-dimensional grid element corresponding to a given voxel, the channel for that atom type within the given voxel is assigned a first value of the binary categorical variable, such as “1”, and when the atom type is not found in the three-dimensional grid element corresponding to the given voxel, the channel for that atom type is assigned a second value of the binary categorical variable, such as “0” within the given voxel.

While there are over 100 elements, most are not encountered in biology. However, even representing the most common biological elements (e.g., H, C, N, O, F, P, S, Cl, Br, I, Li, Na, Mg, K, Ca, Mn, Fe, Co, Zn) may yield 18 channels per voxel or 10,483*18=188,694 inputs to the receptor field. As such, in some embodiments, each respective voxel in a voxel map comprises a plurality of channels, and each channel in the plurality of channels represents a different property that may arise in the three-dimensional space filling polyhedral cell corresponding to the respective voxel. The number of possible channels for a given voxel is even higher in those embodiments where additional characteristics of the atoms (for example, partial charge, presence in ligand versus protein target, electronegativity, or SYBYL atom type) are additionally presented as independent channels for each voxel, necessitating more input channels to differentiate between otherwise-equivalent atoms.

In some embodiments, each voxel has five or more input channels. In some embodiments, each voxel has fifteen or more input channels. In some embodiments, each voxel has twenty or more input channels, twenty-five or more input channels, thirty or more input channels, fifty or more input channels, or one hundred or more input channels. In some embodiments, each voxel has five or more input channels selected from the descriptors found in Table 1 below. For example, in some embodiments, each voxel has five or more channels, each encoded as a binary categorical variable where each such channel represents a SYBYL atom type selected from Table 1 below. For instance, in some embodiments, each respective voxel in a voxel map includes a channel for the C.3 (sp3 carbon) atom type meaning that if the grid in space for a given test object-target object (or training object-target object) complex represented by the respective voxel encompasses an sp3 carbon, the channel adopts a first value (e.g., “1”) and is a second value (e.g. “0”) otherwise.

TABLE 1
SYBYL Atom Types

| SYBYL ATOM TYPE | DESCRIPTION |
|---|---|
| C.3 | sp3 carbon |
| C.2 | sp2 carbon |
| C.ar | aromatic carbon |
| C.1 | sp carbon |
| N.3 | sp3 nitrogen |
| N.2 | sp2 nitrogen |
| N.1 | sp nitrogen |
| O.3 | sp3 oxygen |
| O.2 | sp2 oxygen |
| S.3 | sp3 sulfur |
| N.ar | aromatic nitrogen |
| P.3 | sp3 phosphorous |
| H | hydrogen |
| Br | bromine |
| Cl | chlorine |
| F | fluorine |
| I | iodine |
| S.2 | sp2 sulfur |
| N.pl3 | pl3 trigonal planar |
| | nitrogen |
| LP | lone pair |
| Na | sodium |
| K | potassium |
| Ca | calcium |
| Li | lithium |
| Al aluminum | aluminum |
| Si | silicon |
| N.am | amide nitrogen |
| S.o | sulfoxide sulfur |
| S.o2 | sulfone sulfur |
| N.4 | positively charged |
| | nitrogen |
| O.co2 | oxygen in carboxylate |
| | or phosphate group |
| C.cat | carbocation, used only |
| | in a guadinium group |
| H.spc | hydrogen in SPC water |
| | model |
| O.spc | oxygen in SPC water |
| | model |
| H.t3p | hydrogen in TIP3P |
| | water model |
| O.t3p | oxygen in TIP3P water |
| | model |
| ANY | any atom |
| HEV | heavy (non H) atom |
| HET | heteroatom (N, O, S, P) |
| HAL | halogen |
| Mg | magnesium |
| Cr.oh | hydroxy chromium |
| Cr.th | chromium |
| Se | selenium |
| Fe | iron |
| Cu | copper |
| Zn | zinc |
| Sn | tin |
| Mo | molybdenum |
| Mn | manganese |
| Co.oh | hydroxy cobalt |

In some embodiments, each voxel comprises ten or more input channels, fifteen or more input channels, or twenty or more input channels selected from the descriptors found in Table 1 above. In some embodiments, each voxel includes a channel for halogens.

In some embodiments, a structural protein-ligand interaction fingerprint (SPLIF) score is generated for a pose 48 of a respective compound. In such embodiments, the SPLIF score is used as additional input into the underlying first neural network 72 or is individually encoded in the voxel map. For a description of SPLIFs, see Da and Kireev, 2014, J. Chem. Inf. Model. 54, pp. 2555-2561, “Structural Protein-Ligand Interaction Fingerprints (SPLIF) for Structure-Based Virtual Screening: Method and Benchmark Study,” which is hereby incorporated by reference. A SPLIF implicitly encodes all possible interaction types that may occur between interacting fragments of the compound (test compound or training compound) and the target polymer 38 (e.g., π-π, CH-π, etc.). In the first step, a compound (test compound or training compound)-target polymer 38 is inspected for intermolecular contacts. Two atoms are deemed to be in a contact if the distance between them is within a specified threshold (e.g., within 4.5 Å). For each such intermolecular atom pair, the respective atom of the compounds and target polymer atoms are expanded to circular fragments, e.g., fragments that include the atoms in question and their successive neighborhoods up to a certain distance. Each type of circular fragment is assigned an identifier. In some embodiments, such identifiers are coded in individual channels in the respective voxels. In some embodiments, the Extended Connectivity Fingerprints up to the first closest neighbor (ECFP2) as defined in the Pipeline Pilot software can be used. See, Pipeline Pilot, ver. 8.5, Accelrys Software Inc., 2009, which is hereby incorporated by reference. ECFP retains information about all atom/bond types and uses one unique integer identifier to represent one substructure (e.g., circular fragment). The SPLIF fingerprint encodes all the circular fragment identifiers found. In some embodiments, the SPLIF fingerprint is not encoded in individual voxels but serves as a separate independent input in the neural network discussed below.

In some embodiments, rather than or in addition to SPLIFs, structural interaction fingerprints (SIFt) are computed for each pose of a given compound (test compound or training compound) to a target polymer and independently provided as input into the first neural network 72 or are encoded in the voxel map 52. For a computation of SIFts, see Deng et al., 2003, “Structural Interaction Fingerprint (SIFt): A Novel Method for Analyzing Three-Dimensional Protein-Ligand Binding Interactions,” J. Med. Chem. 47 (2), pp. 337-344, which is hereby incorporated by reference.

In some embodiments, rather than or in addition to SPLIFs and SIFTs, atom-pairs-based interaction fragments (APIFs) are computed for each pose of a given compound (test compound or training compound) with respect to the target polymer 38 and independently provided as input into the first neural network or are individually encoded in the voxel map. For a computation of APIFs, see Perez-Nueno et al., 2009, “APIF: a new interaction fingerprint based on atom pairs and its application to virtual screening,” J. Chem. Inf. Model. 49(5), pp. 1245-1260, which is hereby incorporated by reference.

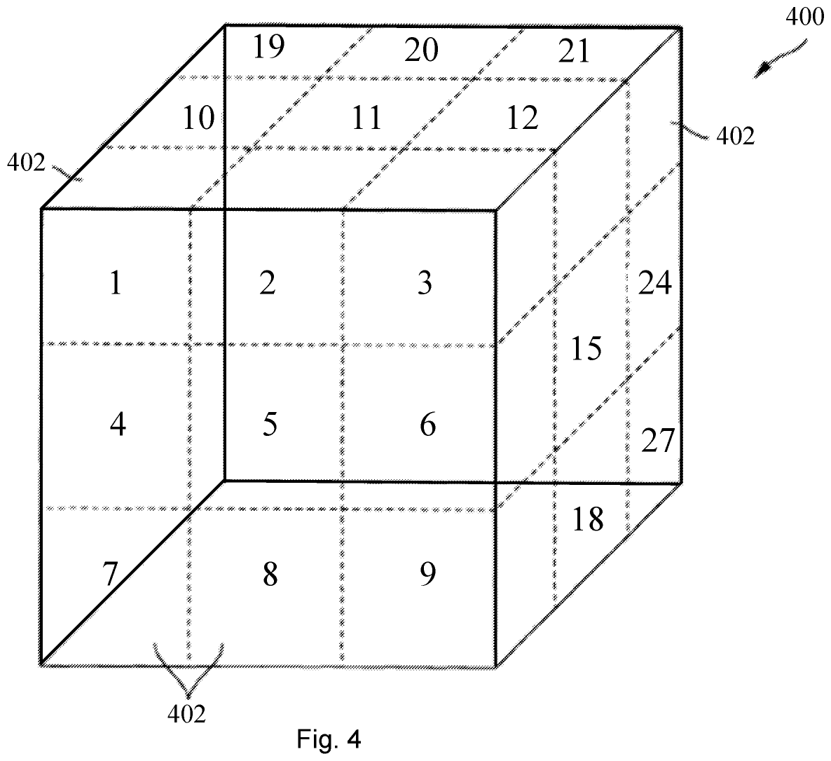

The data representation may be encoded in a way that enables the expression of various structural relationships associated with molecules/proteins for example. The geometric representation may be implemented in a variety of ways and topographies, according to various embodiments. The geometric representation is used for the visualization and analysis of data. For example, in an embodiment, geometries may be represented using voxels laid out on various topographies, such as 2-D, 3-D Cartesian/Euclidean space, 3-D non-Euclidean space, manifolds, etc. For example, FIG. 4 illustrates a sample three-dimensional grid structure 400 including a series of sub-containers, according to an embodiment. Each sub-container 402 may correspond to a voxel. A coordinate system may be defined for the grid, such that each sub-container has an identifier. In some embodiments of the disclosed systems and methods, the coordinate system is a Cartesian system in 3-D space, but in other embodiments of the system, the coordinate system may be any other type of coordinate system, such as a oblate spheroid, cylindrical or spherical coordinate systems, polar coordinates systems, other coordinate systems designed for various manifolds and vector spaces, among others. In some embodiments, the voxels may have particular values associated to them, which may, for example, be represented by applying labels, and/or determining their positioning, among others.

In some embodiments the first neural network 72 is a convolutional neural network that requires a fixed input size. In some such embodiments of the disclosed systems and methods the geometric data (e.g., the voxel map 52 and/or the set of atomic coordinates 49) is cropped to fit within an appropriate bounding box. For example, a cube of 25-40 Å to a side, may be used. In some embodiments in which the compound (test compound or training compound) has been docketed into the active site of target polymer 38, the center of the active site serves as the center of the cube. While in some such embodiments a square cube of fixed dimensions centered on the active site of the target polymer 38 is used to partition the space into the voxel grid, the disclosed systems are not so limited. In some embodiments, any of a variety of shapes is used to partition the space into the voxel grid. In some embodiments, polyhedra, such as rectangular prisms, polyhedra shapes, etc. are used to partition the space. In an embodiment, the grid structure may be configured to be similar to an arrangement of voxels. For example, each sub-structure may be associated with a channel for each atom being analyzed. Also, an encoding method may be provided for representing each atom numerically.

In some embodiments, the voxel map takes into account the factor of time (e.g. along a molecular dynamics run of the compound docked to the target polymer) and may thus be in four dimensions (X, Y, Z, and time).

In some embodiments, other implementations such as pixels, points, polygonal shapes, polyhedrals, or any other type of shape in multiple dimensions (e.g. shapes in 3D, 4D, and so on) may be used instead of voxels.

In some embodiments, the geometric data is normalized by choosing the origin of the X, Y and Z coordinates to be the center of mass of a binding site of the target polymer 38 as determined by a cavity flooding algorithm. For representative details of such algorithms, see Ho and Marshall, 1990, “Cavity search: An algorithm for the isolation and display of cavity-like binding regions,” Journal of Computer-Aided Molecular Design 4, pp. 337-354; and Hendlich et al., 1997, “Ligsite: automatic and efficient detection of potential small molecule-binding sites in proteins,” J. Mol. Graph. Model 15:6, each of which is hereby incorporated by reference. Alternatively, in some embodiments, the origin of the voxel map is centered at the center of mass of the entire co-complex (of the compound docked in the respective pose with respect to the target polymer). In some embodiments, the origin of the voxel map is centered at the center of mass of the compound (test compound or training compound). In some embodiments, the origin of the voxel map is centered at the center of mass of the target polymer 38. The basis vectors may optionally be chosen to be the principal moments of inertia of the entire co-complex, of just the target polymer, or of just the compounds (test compounds or training compounds). In some embodiments, the target polymer 38 has an active site, and the sampling samples the compound (test compound or training compound), in a pose in the active site of the target polymer on the three-dimensional grid basis in which a center of mass of the active site is taken as the origin and the corresponding three-dimensional uniform honeycomb for the sampling represents a portion of the polymer and the compound (test compound or training compound) centered on the center of mass. In some embodiments, the uniform honeycomb is a regular cubic honeycomb and the portion of the target polymer and the compound (test compound or training compound) is a cube of predetermined fixed dimensions. Use of a cube of predetermined fixed dimensions, in such embodiments, ensures that a relevant portion of the geometric data is used and that each voxel map is the same size. In some embodiments, the predetermined fixed dimensions of the cube are N Å×N Å×N Å, where N is an integer or real value between 5 and 100, an integer between 8 and 50, or an integer between 15 and 40. In some embodiments, the uniform honeycomb is a rectangular prism honeycomb and the portion of the target polymer and the compound (test compound or training compound) is a rectangular prism predetermined fixed dimensions Q Å×R Å×S Å, where Q is a first integer between 5 and 100, R is a second integer between 5 and 100, S is a third integer or real value between 5 and 100, and at least one number in the set {Q, R, S} is not equal to another value in the set {Q, R, S}.

In an embodiment, every voxel has one or more input channels, which may have various values associated with them, which in a simple implementation could be on/off, and may be configured to encode for a type of atom. Atom types may denote the element of the atom, or atom types may be further refined to distinguish between other atom characteristics. Atoms present may then be encoded in each voxel. Various types of encoding may be utilized using various techniques and/or methodologies. As an example encoding method, the atomic number of the atom may be utilized, yielding one value per voxel ranging from one for hydrogen to 118 for ununoctium (or any other element).

However, as discussed above, other encoding methods may be utilized, such as “one-hot encoding,” where every voxel has many parallel input channels, each of which is either on or off and encodes for a type of atom. Atom types may denote the element of the atom, or atom types may be further refined to distinguish between other atom characteristics. For example, SYBYL atom types distinguish single-bonded carbons from double-bonded, triple-bonded, or aromatic carbons. For SYBYL atom types, see Clark et al., 1989, “Validation of the General Purpose Tripos Force Field, 1989, J. Comput. Chem. 10, pp. 982-1012, which is hereby incorporated by reference.

In some embodiments, each voxel further includes one or more channels to distinguish between atoms that are part of the target polymer 38 or cofactors versus part of the compound (test compound or training compound). For example, in one embodiment, each voxel further includes a first channel for the target polymer 38 and a second channel for the compound (test compound or training compound). When an atom in the portion of space represented by the voxel is from the target polymer 38, the first channel is set to a value, such as “1”, and is zero otherwise (e.g., because the portion of space represented by the voxel includes no atoms or one or more atoms from the compound). Further, when an atom in the portion of space represented by the voxel is from the compound (test compound or training compound), the second channel is set to a value, such as “1”, and is zero otherwise (e.g., because the portion of space represented by the voxel includes no atoms or one or more atoms from the target polymer 38). Likewise, other channels may additionally (or alternatively) specify further information such as partial charge, polarizability, electronegativity, solvent accessible space, and electron density. For example, in some embodiments, an electron density map for the target polymer overlays the set of three-dimensional coordinates, and the creation of the voxel map further samples the electron density map. Examples of suitable electron density maps include, but are not limited to, multiple isomorphous replacement maps, single isomorphous replacement with anomalous signal maps, single wavelength anomalous dispersion maps, multi-wavelength anomalous dispersion maps, and 2Fo−Fc maps (260). See McRee, 1993, Practical Protein Crystallography, Academic Press, which is hereby incorporated by reference.

In some embodiments, voxel encoding in accordance with the disclosed systems and methods may include additional optional encoding refinements. The following two are provided as examples.

In a first encoding refinement, the required memory may be reduced by reducing the set of atoms represented by a voxel (e.g., by reducing the number of channels represented by a voxel) on the basis that most elements rarely occur in biological systems. Atoms may be mapped to share the same channel in a voxel, either by combining rare atoms (which may therefore rarely impact the performance of the system) or by combining atoms with similar properties (which therefore could minimize the inaccuracy from the combination). In some embodiments, two, three, four, five, six, seven, eight, nine, or ten different atoms share the same channel in a voxel.

An encoding refinement is to have voxels represent atom positions by partially activating neighboring voxels. This results in partial activation of neighboring neurons in the subsequent neural network and moves away from one-hot encoding to a “several-warm” encoding. For example, it may be illustrative to consider a chlorine atom, which has a van der Waals diameter of 3.5 Å and therefore a volume of 22.4 Å³ when a 1 Å³ grid is placed, voxels inside the chlorine atom will be completely filled and voxels on the edge of the atom will only be partially filled. Thus, the channel representing chlorine in the partially-filled voxels will be turned on proportionate to the amount such voxels fall inside the chlorine atom. For instance, if fifty percent of the voxel volume falls within the chlorine atom, the channel in the voxel representing chlorine will be activated fifty percent. This may result in a “smoothed” and more accurate representation relative to the discrete one-hot encoding. Thus, in some embodiments, a characteristic of an atom incurred in the sampling is spread across a subset of voxels in the voxel map and this subset of voxels comprises two or more voxels, three or more voxels, five or more voxels, ten or more voxels, or twenty-five or more voxels. In some embodiments, the characteristic of the atom consists of an enumeration of the atom type (e.g., one of the SYBYL atom types).

Thus, voxelation (rasterization) of the geometric data (the docking of a test or training compound onto a target polymer) that has been encoded is based upon various rules applied to the input data.

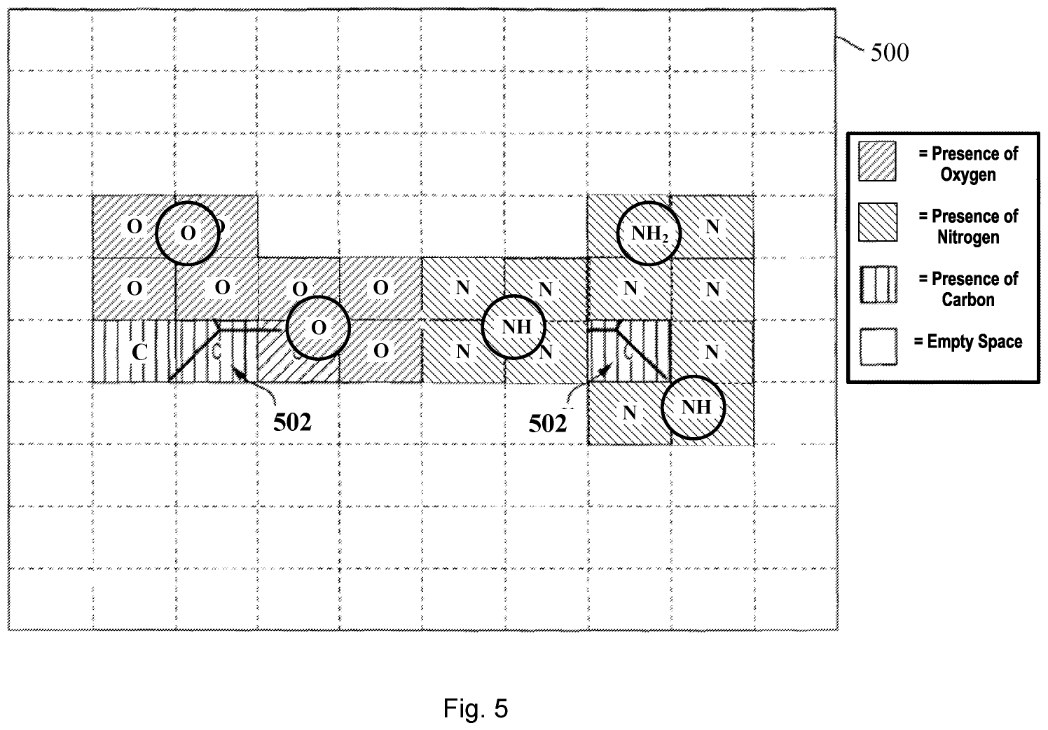

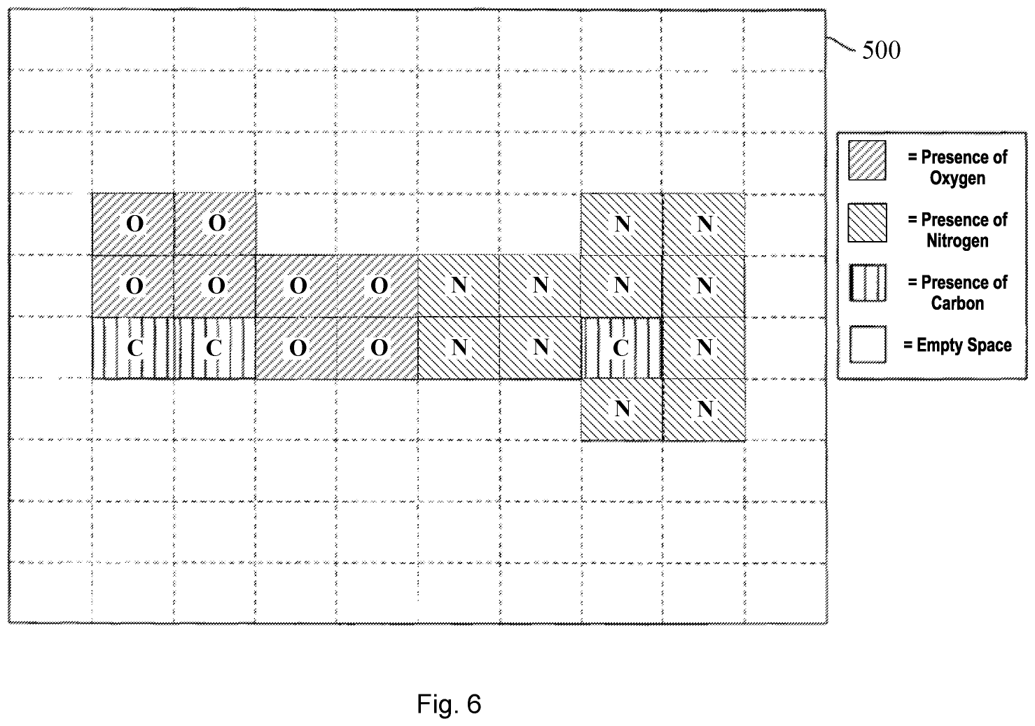

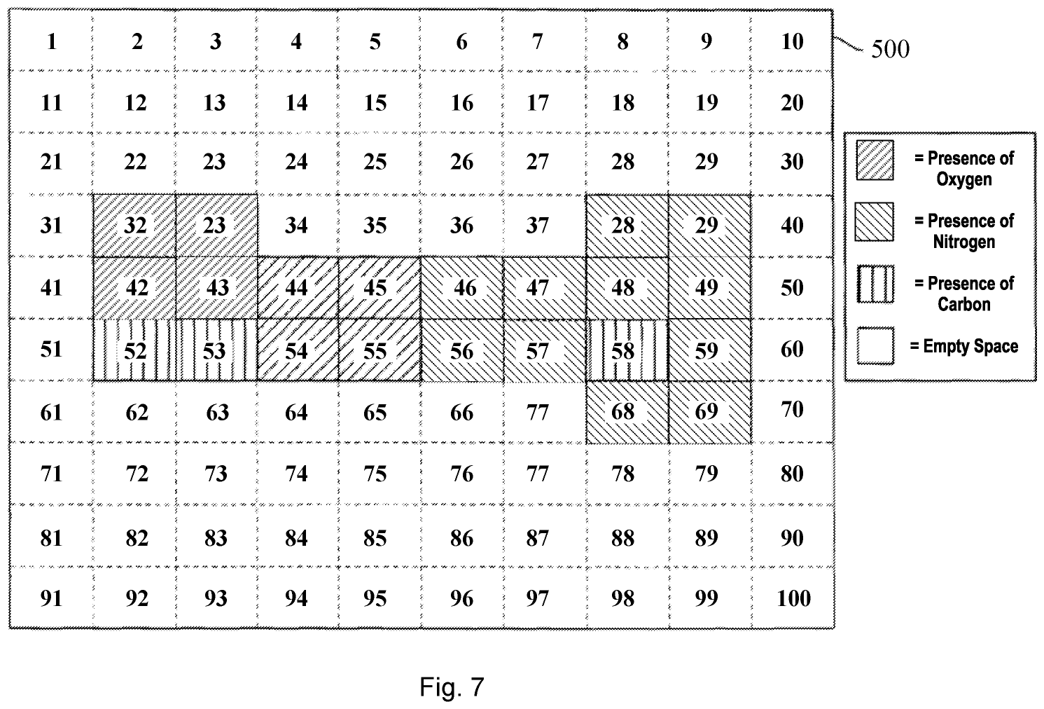

FIGS. 5 and 6 provide views of two compounds 502 encoded onto a two dimensional grid 500 of voxels, according to some embodiments. FIG. 5 provides the two compounds superimposed on the two dimensional grid. FIG. 6 provides the one-hot encoding, using the different shading patterns to respectively encode the presence of oxygen, nitrogen, carbon, and empty space. As noted above, such encoding may be referred to as “one-hot” encoding. FIG. 6 shows the grid 500 of FIG. 5 with the compounds 502 omitted. FIG. 7 provides a view of the two dimensional grid of voxels of FIG. 6, where the voxels have been numbered.

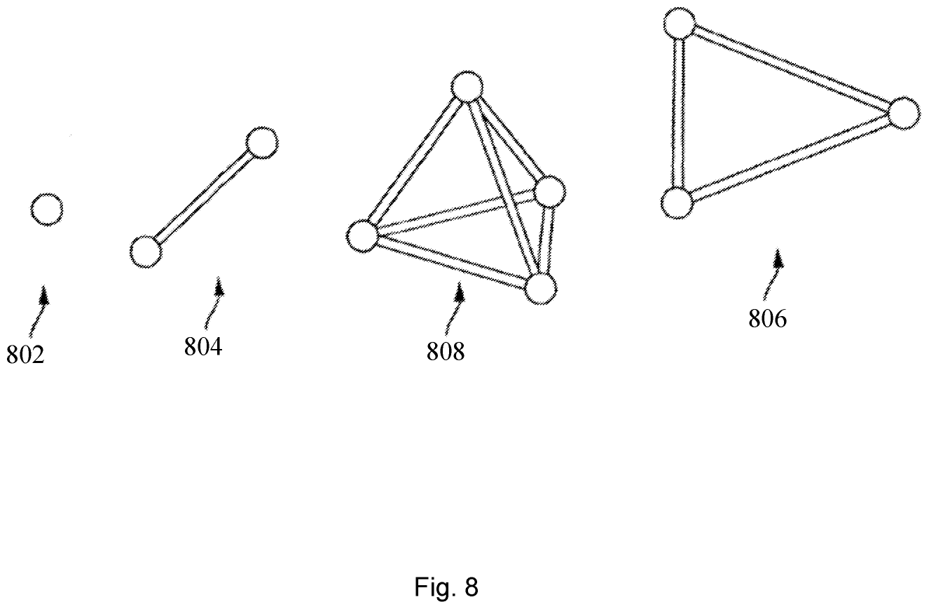

In some embodiments, feature geometry is represented in forms other than voxels. FIG. 8 provides a view of various representations in which features (e.g., atom centers) are represented as 0-D points (representation 802), 1-D points (representation 804), 2-D points (representation 806), or 3-D points (representation 808). Initially, the spacing between the points may be randomly chosen. However, as the predictive model is trained, the points may be moved closer together, or farther apart.

In some embodiments, the input representation for the first neural network 72 can be in the form of 1D-array of features including, but not limited to, three-dimensional coordinates.

Unfolding a voxel map into a corresponding vector. In some embodiments, each voxel map 52 is optionally unfolded into a corresponding vector 54. In some embodiments, each such vector is a one-dimensional vector. For instance, in some embodiments, a cube of 20 Å on each side is centered on the active site of the target polymer 38 with the compound (target compound or training compound) docked in a pose and is sampled with a three-dimensional fixed grid spacing of 1 Å to form corresponding voxels of a voxel map that hold in respective channels basic of the voxel structural features such as atom types as well as, optionally, more complex compound-target polymer descriptors, as discussed above. In some embodiments, the voxels of this three-dimensional voxel map are unfolded into a one-dimensional floating point vector.

In some embodiments, the vectorized representation of voxel maps are input into the first neural network 72. In some embodiments, the vectorized representation of voxel maps are stored in the GPU memory along with the first neural network 72. This provides the advantage of processing the vectorized representation of voxel maps through the first neural network 72 at faster speeds. However, in other embodiments, any or all of the vectorized representations of voxel maps and the first neural network 72 are in memory 92 of system 100 or simply are addressable by system 92 across a network. In some embodiments, any or all of the vectorized representation of voxel maps and the first neural network 72 are in a cloud computing environment.

In some embodiments, each vector 54 is provided to a graphical processing unit memory, where the graphical processing unit memory includes a network architecture that includes a first neural network 72 that is in the form of a convolutional neural network comprising an input layer for sequentially receiving vectors, a plurality of convolutional layers and, optionally, a scorer. The plurality of convolutional layers includes an initial convolutional layer and a final convolutional layer. In some embodiments, the convolutional neural network is not in GPU memory but is in memory 92 of system 100. In some embodiments, the voxel maps 52 are not vectorized before being input into the first neural network 72.

In some embodiments in which the first neural network 72 is a convolutional neural network a convolutional layer in a plurality of convolutional layers within the network comprises a set of learnable filters (also termed kernels). Each filter has fixed three-dimensional size that is convolved (stepped at a predetermined step rate) across the depth, height and width of the input volume of the convolutional layer, computing a dot product (or other functions) between entries (weights, or more generally parameters) of the filter and the input thereby creating a multi-dimensional activation map of that filter. In some embodiments, the filter step rate is one element, two elements, three elements, four elements, five elements, six elements, seven elements, eight elements, nine elements, ten elements, or more than ten elements of the input space. Thus, consider the case in which a filter has size 5′. In some embodiments, this filter will compute the dot product (or other mathematical function) between a contiguous cube of input space that has a depth of five elements, a width of five elements, and a height of five elements, for a total number of values of input space of 125 per voxel channel.

The input space to the initial convolutional layer (e.g., the output from the input layer) is formed from either a voxel map 52 or the vectorized representation 54 of the voxel map. In some embodiments, the vectorized representation of the voxel map is a one-dimensional vectorized representation of the voxel map that serves as the input space to the initial convolutional layer. Nevertheless, when a filter convolves its input space and the input space is a one-dimensional vectorized representation of the voxel map, the filter still obtains from the one-dimensional vectorized representation those elements that represent a corresponding contiguous cube of fixed space in the target polymer 38-compound complex. In some embodiments, the filter uses bookkeeping techniques to select those elements from within the one-dimensional vectorized representation that form the corresponding contiguous cube of fixed space in the target polymer 38-compound complex. Thus, in some instances, this necessarily involves taking a non-contiguous subset of elements in the one-dimensional vectorized representation in order to obtain the element values of the corresponding contiguous cube of fixed space in the target polymer 38-compound complex.

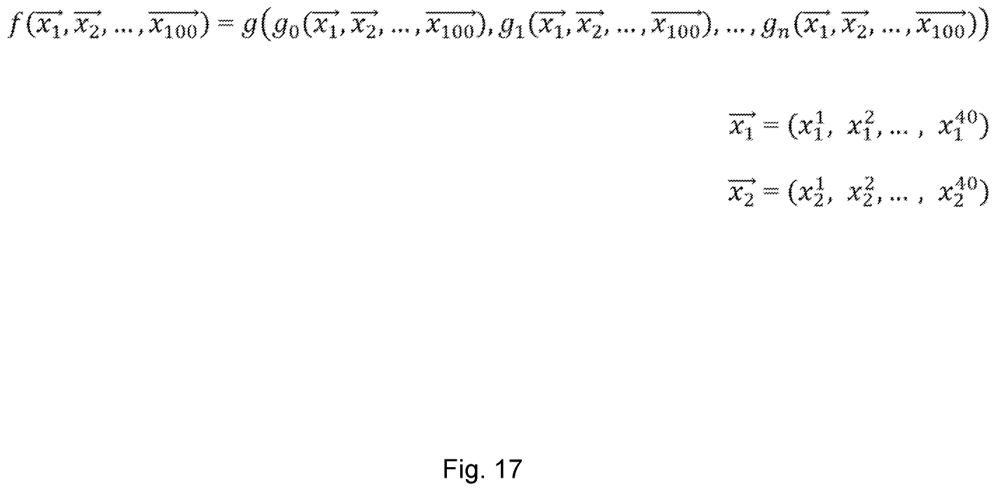

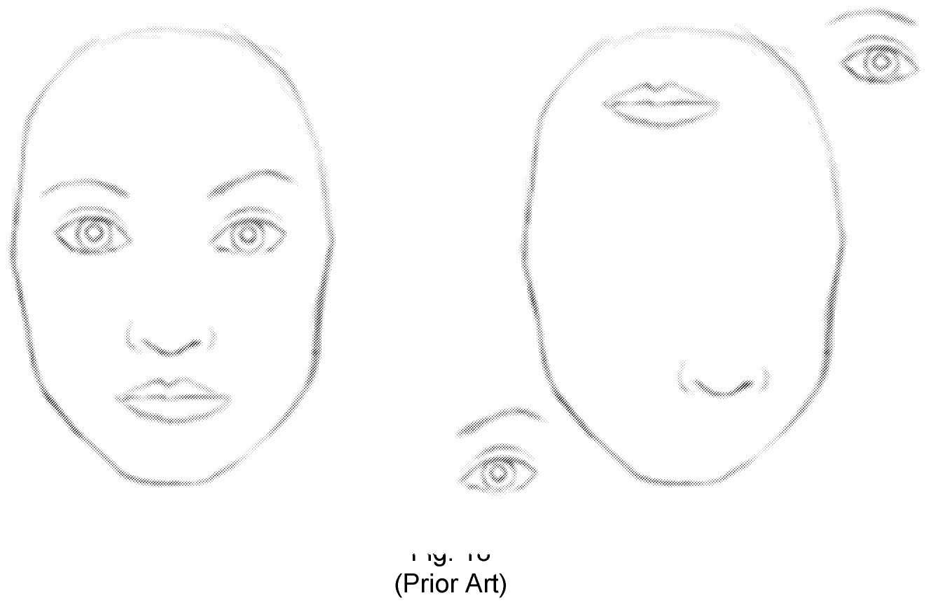

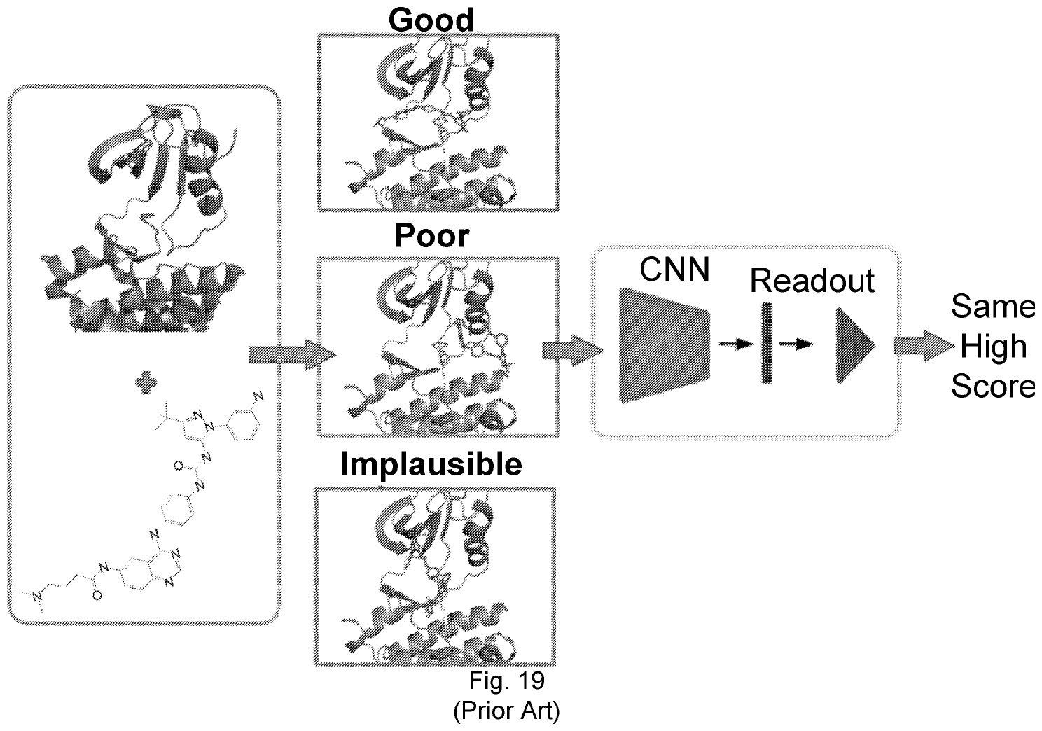

In some embodiments, the filter is initialized (e.g., to Gaussian noise) or trained to have 125 corresponding weights (per input channel) in which to take the dot product (or some other form of mathematical operation such as the function disclosed in FIG. 17) of the 125 input space values in order to compute a first single value (or set of values) of the activation layer corresponding to the filter. In some embodiments the values computed by the filter are summed, weighted, and/or biased. To compute additional values of the activation layer corresponding to the filter, the filter is then stepped (convolved) in one of the three dimensions of the input volume by the step rate (stride) associated with the filter, at which point the dot product (or some other form of mathematical operation such as the mathematical function disclosed in FIG. 17) between the filter weights and the 125 input space values (per channel) is taken at the new location in the input volume is taken. This stepping (convolving) is repeated until the filter has sampled the entire input space in accordance with the step rate. In some embodiments, the border of the input space is zero padded to control the spatial volume of the output space produced by the convolutional layer. In typical embodiments, each of the filters of the convolutional layer canvas the entire three-dimensional input volume in this manner thereby forming a corresponding activation map. The collection of activation maps from the filters of the convolutional layer collectively form the three-dimensional output volume of one convolutional layer, and thereby serves as the three-dimensional (three spatial dimensions) input of a subsequent convolutional layer. Every entry in the output volume can thus also be interpreted as an output of a single neuron (or a set of neurons) that looks at a small region in the input space to the convolutional layer and shares parameters with neurons in the same activation map. Accordingly, in some embodiments, a convolutional layer in the plurality of convolutional layers has a plurality of filters and each filter in the plurality of filters convolves (in three spatial dimensions) a cubic input space of N³ with stride Y, where N is an integer of two or greater (e.g., 2, 3, 4, 5, 6, 7, 8, 9, 10, or greater than 10) and Y is a positive integer (e.g. 1, 2, 3, 4, 5, 6, 7, 8, 9, 10, or greater than 10).

Each layer in the plurality of convolutional layers is associated with a different set of weights, or more generally a different set of parameters. With more particularity, each layer in the plurality of convolutional layers includes a plurality of filters and each filter comprises an independent plurality of parameters (e.g., weights). In some embodiments, a convolutional layer has 128 filters of dimension 5³ and thus the convolutional layer has 128×5×5×5 or 16,000 parameters (e.g., weights) per channel in the voxel map. Thus, if there are five channels in the voxel map, the convolutional layer will have 16,000×5 parameters (e.g., weights), or 80,000 parameters (e.g., weights). In some embodiments some or all such parameters (and, optionally, biases) of every filter in a given convolutional layer may be tied together, e.g. constrained to be identical.

Responsive to input of a respective vector, the input layer feeds a first plurality of values into the initial convolutional layer as a first function of values in the respective vector, where the first function is optionally computed using a graphical processing unit. In some embodiments, the computer system 100 has more than one graphical processing unit and each such graphical processing unit is concurrently used to facilitate the computations of the first neural network 72.

Each respective convolutional layer, other than the final convolutional layer, feeds intermediate values, as a respective second function of (i) the different set of parameters (e.g., weights) associated with the respective convolutional layer and (ii) input values received by the respective convolutional layer, into another convolutional layer in the plurality of convolutional layers. In some embodiments the respective second function is computed using a graphical processing unit. For instance, in some embodiments, each respective filter of the respective convolutional layer canvasses the input volume (in three spatial dimensions) to the convolutional layer in accordance with the characteristic three-dimensional stride of the convolutional layer and at each respective filter position, takes the dot product (or some other mathematical function) of the filter parameters (e.g., weights) of the respective filter and the values of the input volume (contiguous cube that is a subset of the total input space) at the respect filter position thereby producing a calculated point (or a set of points) on the activation layer corresponding to the respective filter position. The activation layers of the filters of the respective convolutional layer collectively represent the intermediate values of the respective convolutional layer.

In some embodiments, the convolutional neural network has one or more activation layers. In some embodiments, the activation layer is a layer of neurons that applies the non-saturating activation function f(x)=max(0, x). It increases the nonlinear properties of the decision function and of the overall network without affecting the receptive fields of the convolutional layer. In other embodiments, the activation layer has other functions to increase nonlinearity, for example, the saturating hyperbolic tangent function f(x)=tanh, f(x)=|tanh(x)|, and the sigmoid function f(x)=(1+e^{-x})⁻¹. Nonlimiting examples of other activation functions found in other activation layers in some embodiments for the neural network may include, but are not limited to, logistic (or sigmoid), softmax, Gaussian, Boltzmann-weighted averaging, absolute value, linear, rectified linear, bounded rectified linear, soft rectified linear, parameterized rectified linear, average, max, min, some vector norm LP (for p=1, 2, 3, . . . , ∞), sign, square, square root, multiquadric, inverse quadratic, inverse multiquadric, polyharmonic spline, and thin plate spline.

The network learns filters within the convolutional layers that activate when they see some specific type of feature at some spatial position in the input. In some embodiments, the initial parameters (e.g., weights) of each filter in a convolutional layer are obtained by training the convolutional neural network against a compound training library. Accordingly, the operation of the convolutional neural network may yield more complex features than the features historically used to conduct binding affinity prediction. For example, a filter in a given convolutional layer of the network that serves as a hydrogen bond detector may be able to recognize not only that a hydrogen bond donor and acceptor are at a given distance and angles, but also recognize that the biochemical environment around the donor and acceptor strengthens or weakens the bond. Additionally, the filters within the network may be trained to effectively discriminate binders from non-binders in the underlying data.

As described above, in some embodiments the first neural network 72 is configured to develop three-dimensional convolutional layers. The input region to the lowest level convolutional layer may be a cube (or other contiguous region) of voxel channels from the receptive field. Higher convolutional layers evaluate the output from lower convolutional layers, while still having their output be a function of a bounded region of voxels that are close together (in 3-D Euclidean distance). In an embodiment, the first neural network 72 is configured to apply regularization techniques to reduce the tendency of the models to overfit the training data.

Zero or more of the network layers in the above-described convolutional neural network may consist of pooling layers. As in a convolutional layer, a pooling layer is a set of functional computations that apply the same function over different spatially-local patches of input. For pooling layers, the output is given by a pooling operators, e.g. some vector norm LP for p=1, 2, 3, . . . , ∞, over several voxels. In some embodiments pooling is done per channel, whereas in other embodiments pooling is done across channels. In some embodiments, pooling partitions the input space into a set of three-dimensional boxes and, for each such sub-region, outputs the maximum or some other mathematical pooling operation such as average pooling. The pooling operation provides a form of translation invariance. The function of the pooling layer is to progressively reduce the spatial size of the representation to reduce the amount of parameters and computation in the network, and hence to also control overfitting. In some embodiments a pooling layer is inserted between successive convolutional layers in the above-described convolutional neural network. Such a pooling layer operates independently on every depth slice of the input and resizes it spatially. In addition to or instead of maximum pooling, the pooling units can also perform other functions, such as average pooling or even L2-norm pooling.

Zero or more of the layers in the above-described convolutional neural network may consist of normalization layers, such as local response normalization or local contrast normalization, which may be applied across channels at the same position or for a particular channel across several positions. These normalization layers may encourage variety in the response of several function computations to the same input.

In some embodiments, the scorer is not present. Rather, the convolutional neural network outputs an initial embedding 74 for an inputted pose 48 rather than a score.

### Use Cases.

The following are sample use cases provided for illustrative purposes only that describe some applications of some embodiments of the present disclosure. Other uses may be considered, and the examples provided below are non-limiting and may be subject to variations, omissions, or may contain additional elements.

Hit discovery. Pharmaceutical companies spend millions of dollars on screening compounds to discover new prospective drug leads. Large compound collections are tested to find the small number of compounds that have any interaction with the disease target of interest. Unfortunately, wet lab screening suffers experimental errors and, in addition to the cost and time to perform the assay experiments, the gathering of large screening collections imposes significant challenges through storage constraints, shelf stability, or chemical cost. Even the largest pharmaceutical companies have only between hundreds of thousands to a few millions of compounds, versus the tens of millions of commercially available molecules, and the hundreds of millions, billions, and even trillions of simulate-able molecules. Examples of databases of commercially available molecules include MCULE (Kiss et al., 2012, “Http://Mcule.Com: A Public Web Service for Drug Discovery,” J. Cheminformatics 4 (1), p. 17) and ENAMINE (Irwin et al., 2016, “Docking Screens for Novel Ligands Conferring New Biology,” J. Med. Chem. 59 (9), pp. 4103-4120).

A potentially more efficient alternative to physical experimentation is virtual high throughput screening. In the same manner that physics simulations can help an aerospace engineer to evaluate possible wing designs before a model is physically tested, computational screening of molecules can focus the experimental testing on a small subset of high-likelihood molecules. This may reduce screening cost and time, reduces false negatives, improves success rates, and/or covers a broader swath of chemical space.

In this application, a protein target may be provided as input to the system. A large set of compounds may also be provided. For each compound, a binding affinity is predicted against the protein target. The resulting scores may be used to rank the compounds, with the best-scoring compounds being most likely to bind the target protein. Optionally, the ranked compounds list is analyzed for clusters of similar compounds; a large cluster may be used as a stronger prediction of compound binding, or compounds may be selected across clusters to ensure diversity in the confirmatory experiments.

Off-target side-effect prediction. Many drugs may be found to have side-effects. Often, these side-effects are due to interactions with biological pathways other than the one responsible for the drug's therapeutic effect. These off-target side-effects may be uncomfortable or hazardous and restrict the patient population in which the drug's use is safe. Off-target side effects are therefore an important criterion with which to evaluate which drug candidates to further develop. While it is important to characterize the interactions of a drug with many alternative biological targets, such tests can be expensive and time-consuming to develop and run. Computational prediction can make this process more efficient.

In applying an embodiment of the present disclosure, a panel of protein targets may be constructed that are associated with significant biological responses and/or side-effects. The system may then be configured to predict binding against each protein target in the panel in turn. Strong activity (that is, activity as potent as compounds that are known to activate the off-target protein) against a particular target may implicate the molecule in side-effects due to off-target effects.

Toxicity prediction. Toxicity prediction is a particularly-important special case of off-target side-effect prediction. Approximately half of drug candidates in late stage clinical trials fail due to unacceptable toxicity. As part of the new drug approval process (and before a drug candidate can be tested in humans), the FDA requires toxicity testing data against a set of targets including the cytochrome P450 liver enzymes (inhibition of which can lead to toxicity from drug-drug interactions) or the hERG channel (binding of which can lead to QT prolongation leading to ventricular arrhythmias and other adverse cardiac effects).

In toxicity prediction, the system may be configured to constrain the off-target proteins to be key antitargets (e.g. CYP450, hERG, or 5-HT_{2B} receptor). The binding affinity for a drug candidate may then be predicted against these proteins. Optionally, the compound may be analyzed to predict a set of metabolites (subsequent molecules generated by the body during metabolism/degradation of the original compound), which can also be analyzed for binding against the antitargets. Problematic compounds may be identified and modified to avoid the toxicity or development on the molecular series may be halted to avoid wasting additional resources.

Potency optimization. One of the key requirements of a drug candidate is strong binding against its disease target. It is rare that a screen will find compounds that bind strongly enough to be clinically effective. Therefore, initial compounds seed a long process of optimization, where medicinal chemists iteratively modify the molecular structure of compounds to propose new compounds with increased strength of target binding. Each new compound is synthesized and tested, to determine whether the changes successfully improved binding. The system may be configured to facilitate this process by replacing physical testing with computational prediction.

In this application, the disease protein target and a set of lead compounds may be input into the system. The system may be configured to produce binding affinity predictions for the set of leads. Optionally, the system could highlight differences between the candidate compounds that could help inform the reasons for the predicted differences in binding affinity. The medicinal chemist user can use this information to propose a new set of compounds with, hopefully, improved activity against the target. These new alternative compounds may be analyzed in the same manner.

Selectivity optimization. As discussed above, compounds tend to bind a host of proteins at a variety of strengths. For example, the binding pockets of protein kinases (which are popular chemotherapy and inflammation targets) are very similar and most kinase inhibitors affect many different kinases. This means that various biological pathways are simultaneously modified, which yields a “dirty” medicinal profile and many side-effects. The critical challenge in the design of many drugs, therefore, is not activity per se but specificity: the ability to selectively target one protein (or a subset of proteins) from a set of possibly-closely related proteins.

The system can reduce the time and cost of optimizing the selectivity of a candidate drug. In this application, a user may input two sets of target proteins. One set describes target proteins against which the compound should be active, while the other set describes target proteins against which the compound should be inactive. The system may be configured to make predictions for the compound against all of the proteins in both sets, establishing a profile of interaction strengths. Optionally, these profiles could be analyzed to suggest explanatory patterns in the target proteins. The user can use the information generated by the system to consider structural modifications to a compound that would improve the relative binding to the different target protein sets, and to design new candidate compounds with better specificity. Optionally, the system could be configured to highlight differences between the candidate compounds that could help inform the reasons for the predicted differences in selectivity. The proposed candidates can be analyzed iteratively, to further refine the specificity of their activity profiles.

Fitness function for automated molecular design. Automated tools to perform the preceding optimizations are valuable. A successful drug compound requires optimization and balance among potency, selectivity, and toxicity. “Scaffold hopping” (when the activity of a lead compound is preserved but the chemical structure is significantly altered) can yield improved pharmacokinetics, pharmacodynamics, toxicity, or intellectual property profiles. Algorithms exist to iteratively suggest new compounds, such as random generation of compounds, growth of molecular fragments to fill a given binding site, genetic algorithms to “mutate” and “cross-breed” a population of compounds, and swapping of pieces of a compound with bioisosteric replacements. The compounds generated by each of these methods can be evaluated against the multiple objectives described above (potency, selectivity, toxicity) and, in the same way that the technology can be informative on each of the preceding manual settings (binding prediction, selectivity, side-effect and toxicity prediction), it can be incorporated in an automated compound design system.

Drug repurposing. Drugs typically have side-effects and, from time to time, these side-effects are beneficial. For instance aspirin, which is generally used as a headache treatment, is also taken for cardiovascular health. Drug repositioning can significantly reduce the cost, time, and risk of drug discovery because the drugs have already been shown to be safe in humans and have been optimized for rapid absorption and favorable stability in patients. Unfortunately, drug repositioning has been largely serendipitous. For example, sildenafil (Viagra), was developed as a hypertension drug and was unexpectedly observed to be an effective treatment for erectile dysfunction. Computational prediction of off-target effects can be used in the context of drug repurposing to identify compounds that could be used to treat alternative diseases.

In the present disclosure, as in off-target side-effect prediction, the user may assemble a set of possible target proteins, where each target protein is linked to a disease. That is, inhibition of each target protein would treat a (possibly different) disease; for example, inhibitors of Cyclooxygenase-2 can provide relief from inflammation, whereas inhibitors of Factor Xa can be used as anticoagulants. These target proteins are annotated with the binding affinity of approved drugs, if any exist. A set of compounds is then assembled, restricting the set to compounds that have been approved or investigated for use in humans. Finally, for each pair of target protein and compound, the user may use the system to predict the binding affinity. Candidates for drug repurposing may be identified if the predicted binding affinity of the molecule is close to the binding affinity of effective drugs for the protein.

Drug resistance prediction. Drug resistance is an inevitable outcome of pharmaceutical use, which puts selection pressure on rapidly dividing and mutating pathogen populations. Drug resistance is seen in such diverse disease agents as viruses (HIV), exogenous microorganisms (MRSA), and disregulated host cells (cancers). Over time, a given medicine will become ineffective, irrespective of whether the medicine is an antibiotic or a chemotherapy. At that point, the intervention can shift to a different medicine that is, hopefully, still potent. In HIV, there are well-known disease progression pathways that are defined by which mutations the virus will accumulate while the patient is being treated.

There is considerable interest in predicting how disease agents adapt to medical intervention. One approach is to characterize which mutations will occur in the disease agent while under treatment. Specifically, the protein target of a medicine needs to mutate so as to avoid binding the drug while simultaneously continuing to bind its natural substrate.

In this application, a set of possible mutations in the target protein may be proposed. For each mutation, the resulting protein shape may be predicted. For each of these mutant protein forms, the system may be configured to predict a binding affinity for both the natural substrate and the drug. The mutations that cause the protein to no longer bind to the drug but also to continue binding to the natural substrate are candidates for conferring drug resistance. These mutated proteins may be used as targets against which to design drugs, e.g. by using these proteins as inputs to one of these other prediction use cases.

Personalized medicine. Ineffective medicines should not be administered. In addition to the cost and hassle, all medicines have side-effects. Moral and economic considerations make it imperative to give medicines only when the benefits outweigh these harms. It may be important to be able to predict when a medicine will be useful. People differ from one another by a handful of mutations. However, small mutations may have profound effects. When these mutations occur in the disease target's active (orthosteric) or regulatory (allosteric) sites, they can prevent the drug from binding and, therefore, block the activity of the medicine. When a particular person's protein structure is known (or predicted), the system can be configured to predict whether a drug will be effective or the system may be configured to predict when the drug will not work.

For this application, the system may be configured to receive as input the drug's chemical structure and the specific patient's particular expressed protein. The system may be configured to predict binding between the drug and the protein and, if the drug's predicted binding affinity that particular patient's protein structure is too weak to be clinically effective, clinicians or practitioners may prevent that drug from being fruitlessly prescribed for the patient.

Drug trial design. This application generalizes the above personalized medicine use case to the case of patient populations. When the system can predict whether a drug will be effective for a particular patient phenotype, this information can be used to help design clinical trials. By excluding patients whose particular disease targets will not be sufficiently affected by a drug, a clinical trial can achieve statistical power using fewer patients. Fewer patients directly reduces the cost and complexity of clinical trials.

For this application, a user may segment the possible patient population into subpopulations that are characterized by the expression of different proteins (due to, for example, mutations or isoforms). The system may be configured to predict the binding strength of the drug candidate against the different protein types. If the predicted binding strength against a particular protein type indicates a necessary drug concentration that falls below the clinically-achievable in-patient concentration (as based on, for example, physical characterization in test tubes, animal models, or healthy volunteers), then the drug candidate is predicted to fail for that protein subpopulation. Patients with that protein may then be excluded from a drug trial.

Agrochemical design. In addition to pharmaceutical applications, the agrochemical industry uses binding prediction in the design of new pesticides. For example, one consideration for pesticides is that they stop a single species of interest, without adversely impacting any other species. For ecological safety, a person could desire to kill a weevil without killing a bumblebee.

For this application, the user could input a set of target protein structures, from the different species under consideration, into the system. A subset of target proteins could be specified as the target proteins against which to be active, while the rest would be specified as target proteins against which the compounds should be inactive. As with previous use cases, some set of compounds (whether in existing databases or generated de novo) would be considered against each target protein, and the system would specify the compounds having maximal effectiveness against the first group of target proteins while avoiding the second group of target proteins.

Materials science. To predict the behavior and properties of new materials, it may be useful to analyze molecular interactions. For example, to study solvation, the user may input a repeated crystal structure of a given small molecule and assess the binding affinity of another instance of the small molecule on the crystal's surface. To study polymer strength, a set of polymer strands may be input analogously to a protein target structure, and an oligomer of the polymer may be input as a small molecule. Binding affinity between the polymer strands may therefore be predicted by the system.

Simulation. Simulators often measure the binding affinity of a compound to a protein, because the propensity of a compound to stay in a region of the target protein correlates to its binding affinity there. An accurate description of the features governing binding could be used to identify regions and poses that have particularly high or low binding energy. The energetic description can be folded into molecular dynamic simulations to describe the motion of a molecule and the occupancy of the protein binding region. Similarly, stochastic simulators for studying and modeling systems biology could benefit from an accurate prediction of how small changes in compound concentrations impact biological networks.

### Examples

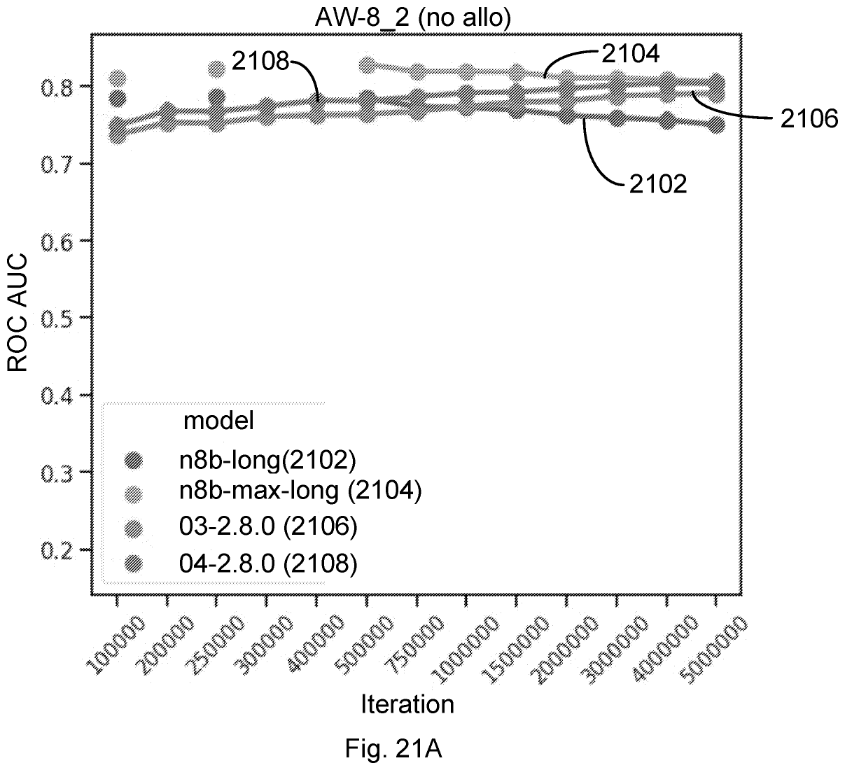

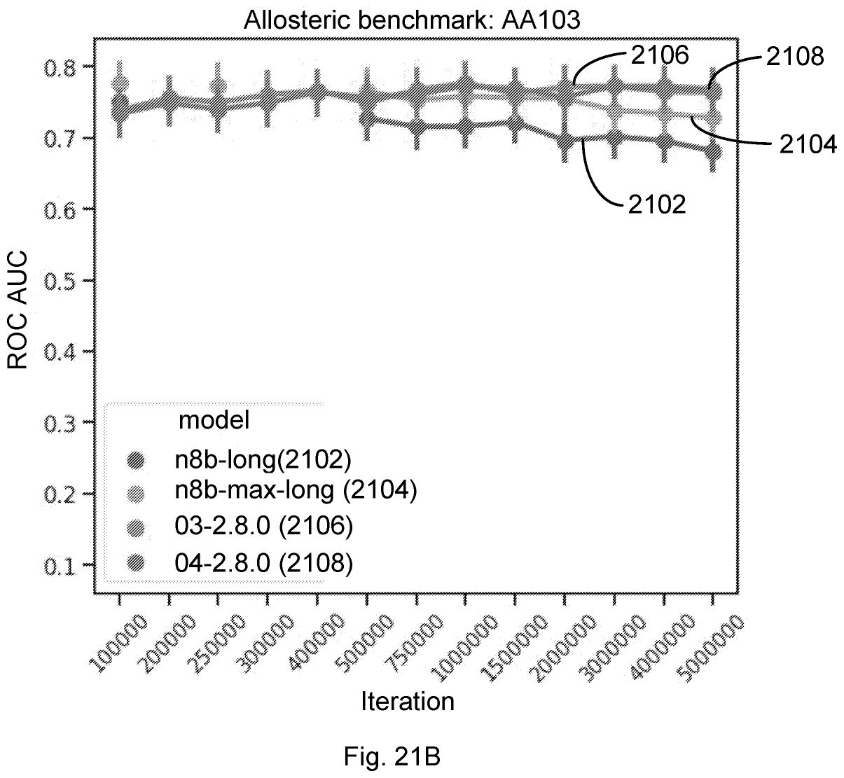

FIG. 21A provides area under the curve (AUC) receiver operator characteristic (ROC) as a function of training iterations for two architectures of the present disclosure, o3-2.8.0 (2106) and o4-2.8.0 (2108), illustrated in FIG. 9B, relative to two other deep learning neural networks, architectures n8b-long (2102) and n8b-max-long (2104) The n8b-long and n8b-max-long architectures are of the type disclosed in U.S. Provisional Patent Application No. 63/251,142 entitled “Characterization of Interactions Between Compounds and Polymers Using Negative Pose Data and Model Conditioning,” filed Oct. 1, 2021, which is hereby incorporated by reference, which requires poses for each training compound that include a “positive pose” and a “negative pose”. The architectures n8b-long and n8b-max-long don't apply an attention mechanism 77 to a plurality of initial embeddings 74 that each represent a different pose 48 of a compound in order to obtain an attention embedding 79 while the architectures o3-2.8.0 and o4-2.8.0 do. For the calculations summarized in FIG. 21A, there were 3,097 different target proteins utilized and a library of 5,562,818 compound-protein pairs available for model training. All 3,097 different target proteins were used in this training. The 3,097 different target proteins were deemed to be non-allosteric. As indicated above with reference to FIG. 10, each of the four models (n8b-long, n8b-max-ling, o3-2.8.0, and o4-2.8.0) was trained using respective minibatches of compound-protein pairs, each minibatch of compound-protein pairs selected from among the compound-protein pairs available, using graphical processing units (GPUs), where the number of compound-protein pairs in a minibatch was limited by GPU memory size. For the training referenced in FIG. 21A, each minibatch consisted of poses for 64 different compound-protein pairs. Each compound used in training had a known binary activity label with respect to at least some of the 3,097 target proteins, either “active” when the pKa for the compound with respect to the target protein was less than 10 μM, or “inactive” when the pKa for the compound with respect to the target protein was greater than 10 μM. Thus, each compound used in the training summarized in FIG. 21A consisted of activity labels for at least some of the 3097 target proteins. During the training summarized in FIG. 21A, if the activity of a particular training compound against a particular target protein was not known, the corresponding compound-protein pair was not included in the training. For each respective compound-protein pair of a respective minibatch, the error exhibited by a respective model in computing the activity label for the respective training compound to the paired target protein pair of the respective compound-protein pair was used to refine the parameters of the respective model as part of an interaction. Thus, each iteration represented the training arising from all the compound-protein pairs of a respective minibatch. As illustrated in FIG. 21A, ROC AUC performance of each model in predicting the activity of training compounds in compound-protein pairs is given from 100000 to 5,000,000 such iterations. FIG. 21A shows that the ROC AUC statistics of the models of the present disclosure, represented by curves 2106 and 2108, consistently improve as the number of iterations increases, whereas the ROC AUC statistics of models n8b-long and n8b-max-long, represented by curves 2102 and 2104, exhibit overtraining such that their ROC AUC value decrease after about 500000 iterations.

FIG. 21B provides AUC ROC statistics for two architectures of the present disclosure, o3-2.8.0 (2106) and o4-2.8.0 (2108), relative to two other deep learning neural networks, architectures-long (2102) and n8b-max-long (2104) against the allosteric benchmark AA103. FIG. 21B shows that o3-2.8.0 and o4-2.8.0 have improved AUC ROC statistics relative to architectures-long (2102) and n8b-max-long (2104) against the allosteric benchmark AA103. For the calculations summarized in FIG. 21B, there were 103 different target proteins utilized and a library of 552,011 compound-protein pairs available for model training. All 103 different target proteins were used in this training. The 103 different target proteins were deemed to be allosteric. As indicated above with reference to FIG. 21A, each of the four models (n8b-long, n8b-max-long, o3-2.8.0, and o4-2.8.0) was trained using respective minibatches of training compound-protein pairs, each minibatch of compound-protein pairs selected from among the training compound-protein pairs available, using graphical processing units (GPUs), where the number of compound-protein pairs in a minibatch was limited by GPU memory size. For the training referenced in FIG. 21B, each minibatch consisted of all the poses for 64 different compound-protein pairs. Each training compound used in the model training had a known binary activity label with respect to at least some of the 103 target proteins, either “active” when the pKa for the training compound with respect to the target protein was less than 10 μM, or “inactive” when the pKa for the training compound with respect to the target protein was greater than 10 μM. Thus, each training compound used in the training summarized in FIG. 21B consisted of activity labels for at least some of the 103 target proteins. During the training summarized in FIG. 21B, if the activity of a particular training compound against a particular target protein was not known, the corresponding compound-protein pair was not included in the training. For each respective compound-protein pair of a respective minibatch, the error in computing the activity label for the respective training compound to the paired target protein pair of the respective compound-protein pair was used to refine model parameters as part of an interaction. Thus, each iteration represented the training arising from all the compound-protein pairs of a respective minibatch. As illustrated in FIG. 21B, ROC AUC performance is given from 100000 to 5,000,000 such iterations. FIG. 21B shows that the ROC AUC statistics of the models of the present disclosure, represented by curves 2106 and 2108, consistently improve as the number of iterations increases, whereas the ROC AUC statistics of models n8b-long and n8b-max-long, represented by curves 2102 and 2104, exhibit overtraining as the number of training iterations increase.

### CONCLUSION

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A computer system for characterizing an interaction between a test compound and a target polymer, the computer system comprising:
one or more processors; and
memory addressable by the one or more processors, the memory storing at least one program for execution by the one or more processors, the at least one program comprising instructions for:
(A) obtaining a plurality of sets of atomic coordinates, wherein each respective set of atomic coordinates in the plurality of sets of atomic coordinates comprises the test compound bound to the target polymer in a corresponding pose in a plurality of poses, wherein each respective set of atomic coordinates in the plurality of sets of atomic coordinates comprises atomic coordinates for at least 5 atoms;
(B) for each respective set of atomic coordinates in the plurality of sets of atomic coordinates, inputting the respective set of atomic coordinates or an encoding of the respective set of atomic coordinates into a first neural network, to obtain a corresponding initial embedding as output of the first neural network, thereby obtaining a plurality of initial embeddings, wherein each initial embedding in the plurality of initial embeddings corresponds to a set of atomic coordinates in the plurality of sets of atomic coordinates and wherein the first neural network comprises more than 400 parameters and performs at least 10,000 computations to compute each initial embedding in the plurality of initial embeddings;
(C) applying an attention mechanism to the plurality of initial embeddings, in concatenated form, thereby obtaining an attention embedding;
(D) applying a pooling function to the attention embedding to derive a pooled embedding; and
(E) inputting the pooled embedding into a first model thereby obtaining a first interaction score of an interaction between the test compound and the target polymer.

2. The computer system of claim 1, wherein the first interaction score represents a binding coefficient of the test compound to the target polymer.

3. The computer system of claim 2, wherein the binding coefficient is an IC₅₀, EC₅₀, Kd, KI, or pKI for the test compound with respect to the target polymer.

4. The computer system of claim 1, wherein the first interaction score represents an in silico pose quality score of the test compound to the target polymer.

5. The computer system of any one of claims 1-4, wherein the first model is a fully connected second neural network.

6. The computer system of claim 1, wherein the at least one program further comprises instructions for inputting the pooled embedding into a second model thereby obtaining a second interaction score of an interaction between the test compound and the target polymer, wherein
the first model is a first fully connected neural network,
the second model is a second fully connected neural network,
the first interaction score represents an in silico pose quality score of the test compound to the target polymer, and
the second interaction score represents an in silico pose quality score of the test compound to the target polymer.

7. The computer system of claim 6, wherein the at least one program further comprises instructions for inputting the first interaction score and the second interaction score into a third model to obtain a third interaction score, wherein the third model is a third fully connected neural network.

8. The computer system of claim 7, wherein the third interaction score is discrete-binary activity score with a first value when the test compound is determined by third model to be inactive and a second value when the test compound is determined by the third model to be active.

9. The computer system of any one of claims 1-8, wherein the target polymer is a protein, a polypeptide, a polynucleic acid, a polyribonucleic acid, a polysaccharide, or an assembly of any combination thereof.

10. The computer system of any one of claims 1-9, wherein each set of atomic coordinates in the plurality of sets of atomic coordinates comprises three-dimensional coordinates {x₁, . . . x_{N}} for at least a portion of the polymer from a crystal structure of the target polymer resolved at a resolution of 2.5 Å or better or a resolution of 3.3 Å or better.

11. The computer system of any one of claims 1-9, wherein each set of atomic coordinates in the plurality of sets of atomic coordinates comprises an ensemble of three-dimensional coordinates for at least a portion of the target polymer determined by nuclear magnetic resonance, neutron diffraction, or cryo-electron microscopy.

12. The computer system of claim 1, wherein the first interaction score is a binary score, wherein
a first value for the binary score represents an IC₅₀, EC₅₀, Kd, KI, or pKI for the test compound with respect to the target polymer that is above a first threshold, and
a second value for the binary score represents an IC₅₀, EC₅₀, Kd, KI, or pKI for the test compound with respect to the target polymer that is below the first threshold.

13. The computer system of any one of claims 1-12, wherein the test compound satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

14. The computer system of any one of claims 1-13, wherein the test compound is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons.

15. The computer system of any one of claims 1-13, wherein the test compound is an organic compound having a molecular weight of between 400 Daltons and 10000 Daltons.

16. The computer system of any one of claims 1-15, wherein the plurality of sets of atomic coordinates consists of between 2 and 64 poses.

17. The computer system of any one of claims 1-16, wherein the first neural network is a convolutional neural network.

18. The computer system of claim 17, wherein each respective set of atomic coordinates in the plurality of sets of atomic coordinates comprises atomic coordinates for at least 80 atoms.

19. The computer system of any one of claims 1-16, wherein the first neural network is a graph neural network.

20. The computer system of claim 19, wherein the graph neural network is characterized by an initial embedding layer and a plurality of interaction layers that each contribute an interaction data structure, in a plurality of interaction data structures, for each atom in the respective set of atomic set of atomic coordinates for the corresponding pose in the plurality of poses that are pooled to form the corresponding initial embedding for the corresponding pose.

21. The computer system of any one of claims 1-16, wherein the first neural network is a equivariant neural network or a message passing neural network.

22. The computer system of any one of claims 1-16, wherein the first neural network comprises a plurality of graph convolutional blocks and each block considers connectivity within the respective set of atomic coordinates using a plurality of radial graphs.

23. The computer system of any one of claims 1-22, wherein the first neural network comprises 1×10⁶ parameters.

24. The computer system of any one of claims 1-23, wherein the corresponding initial embedding comprises a data structure comprising 100 or more values.

25. The computer system of any one of claims 1-24, wherein the plurality of initial embeddings comprises a first plurality of values, and wherein the applying the attention mechanism comprises:
(i) inputting the first plurality of values into an attention neural network thereby obtaining a first plurality of weights, wherein each weight in the first plurality of weights corresponds to a respective value in the first plurality of values, and
(ii) weighting each respective value in the first plurality of values by the corresponding weight in the plurality of weights thereby obtaining the attention embedding.

26. The computer system of claim 25, wherein the first plurality of weights sum to one and each weight in the first plurality of weights is a scalar value between zero and one.

27. The computer system of any one of claims 1-26, wherein the pooling function collapses the attention embedding into the pooled embedding by applying a statistical function to combine each portion of the attention embedding representing a different pose in the plurality of poses to form the pooled embedding.

28. The computer system of claim 27, wherein the attention embedding includes a corresponding plurality of values for a corresponding plurality of elements for each respective pose in the plurality of poses and the statistical function is a maximum function that takes a maximum value across corresponding elements of each respective pose represented in the attention embedding to form the pooled embedding.

29. The computer system of claim 27, wherein the attention embedding includes a corresponding plurality of values for a corresponding plurality of elements for each respective pose in the plurality of poses and the statistical function is an average function that averages the corresponding elements of each respective pose represented in the attention embedding to form the pooled embedding.

30. The computer system of claim 1, wherein the first model is a regression task and the first interaction score quantifies the interaction between the test compound and the target polymer.

31. The computer system of claim 1, wherein the first model is a classification task and the first interaction score classifies the interaction between the test compound and the target polymer.

32. The computer system of any one of claims 1-31, wherein each respective set of atomic coordinates in the plurality of sets of atomic coordinates comprises atomic coordinates for at least 15 atoms, at least 20 atoms, at least 25 atoms, or at least 30 atoms.

33. The computer system of any one of claims 1-32, wherein the first neural network performs at least 100,000 computations to compute each initial embedding in the plurality of initial embeddings.

34. The computer system of any one of claims 1-32, wherein the first neural network performs at least 1×10⁶ computations to compute each initial embedding in the plurality of initial embeddings.

35. The computer system of any one of claims 1-34, wherein the first model performs at least 10,000 computations to compute the first interaction score.

36. The computer system of any one of claims 1-34, wherein the first model performs at least 100,000 computations to compute the first interaction score.

37. The computer system of any one of claims 1-34, wherein the first model performs at least 1×10⁶ computations to compute the first interaction score.

38. The computer system of any one of claims 1-37, wherein the first model comprises more than 400 parameters and wherein the first model performs more than 1000 computations to compute the first interaction score.

39. The computer system of any one of claims 1-37, wherein the first model comprises more than 400 parameters and wherein the first model performs more than 10,000 computations to compute the first interaction score.

40. A method for characterizing an interaction between a test compound and a target polymer, the method comprising:
(A) obtaining a plurality of sets of atomic coordinates, wherein each respective set of atomic coordinates in the plurality of sets of atomic coordinates comprises the test compound bound to the target polymer in a corresponding pose in a plurality of poses, wherein each respective set of atomic coordinates in the plurality of sets of atomic coordinates comprises atomic coordinates for at least 5 atoms;
(B) for each respective set of atomic coordinates in the plurality of sets of atomic coordinates, inputting the respective set of atomic coordinates or an encoding of the respective set of atomic coordinates into a first neural network, to obtain a corresponding initial embedding as output of the first neural network, thereby obtaining a plurality of initial embeddings, wherein each initial embedding in the plurality of initial embeddings corresponds to a set of atomic coordinates in the plurality of sets of atomic coordinates and wherein the first neural network comprises more than 400 parameters and performs at least 10,000 computations to compute each initial embedding in the plurality of initial embeddings;
(C) applying an attention mechanism to the plurality of initial embeddings, in concatenated form, thereby obtaining an attention embedding;
(D) applying a pooling function to the attention embedding to derive a pooled embedding; and
(E) inputting the pooled embedding into a first model thereby obtaining a first interaction score of an interaction between the test compound and the target polymer.

41. A non-transitory computer readable storage medium, wherein the non-transitory computer readable storage medium stores instructions, which when executed by a computer system, cause the computer system to perform a method for characterizing an interaction between a test compound and a target polymer, the method comprising:
(A) obtaining a plurality of sets of atomic coordinates, wherein each respective set of atomic coordinates in the plurality of sets of atomic coordinates comprises the test compound bound to the target polymer in a corresponding pose in a plurality of poses, wherein each respective set of atomic coordinates in the plurality of sets of atomic coordinates comprises atomic coordinates for at least 5 atoms;
(B) for each respective set of atomic coordinates in the plurality of sets of atomic coordinates, inputting the respective set of atomic coordinates or an encoding of the respective set of atomic coordinates into a first neural network, to obtain a corresponding initial embedding as output of the first neural network, thereby obtaining a plurality of initial embeddings, wherein each initial embedding in the plurality of initial embeddings corresponds to a set of atomic coordinates in the plurality of sets of atomic coordinates and wherein the first neural network comprises more than 400 parameters and performs at least 10,000 computations to compute each initial embedding in the plurality of initial embeddings;
(C) applying an attention mechanism to the plurality of initial embeddings, in concatenated form, thereby obtaining an attention embedding;
(D) applying a pooling function to the attention embedding to derive a pooled embedding; and
(E) inputting the pooled embedding into a first model thereby obtaining a first interaction score of an interaction between the test compound and the target polymer.
